(19) United States
(12) Patent Application Publication
(10) Pub. No.: US 2008/0070930 A1
(43) Pub. Date: Mar. 20, 2008

(54) Pyrimidine and Pyrazine Derivatives

(76) Inventors: Kenneth He Huang, Chapel Hill, NC (US); Emilie D. Smith, Apex, NC (US); James Veal, Apex, NC (US)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 11/844,456

(22) Filed: Aug. 24, 2007

Related U.S. Application Data
(60) Provisional application No. 60/823,428, filed on Aug. 24, 2006.

(51) Int. Cl. C07D 403/04; A61K 31/497; A61K 31/506; C07D 403/14; A61P 31/00; A61P 35/00

(52) U.S. Cl. 514/255.05; 514/275; 544/324; 544/405

(57) ABSTRACT

Disclosed are compounds and pharmaceutically acceptable salts of Formula I 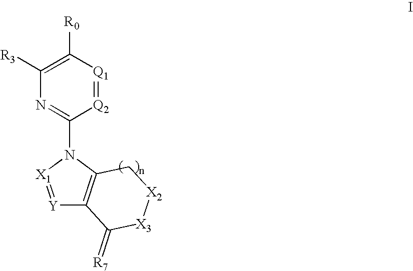
wherein R₀, R₃, R₇, n, Q₁, Q₂, Y, and X₁-X₃ are as defined herein. Compounds of Formula I are useful in the treatment of diseases and/or conditions related to cell proliferation, such as cancer, inflammation, arthritis, angiogenesis, or the like. Also disclosed are pharmaceutical compositions comprising compounds of the invention and methods of treating the aforementioned conditions using such compounds.

### BACKGROUND OF THE INVENTION

1. Field of the Invention

The invention relates to pyrimidine and pyrazine derivatives and more specifically to such compounds that are useful in the treatment and/or prevention of diseases and/or conditions related to cell proliferation, such as cancer, inflammation and inflammation-associated disorders, and conditions associated with angiogenesis. Compounds of the invention are also useful in the treatment and/or prevention of infectious diseases, in particular, fungal and viral infections.

2. Description of the Related Art

Cancer is characterized by abnormal cellular proliferation. Cancer cells exhibit a number of properties that make them dangerous to the host, typically including an ability to invade other tissues and to induce capillary ingrowth, which assures that the proliferating cancer cells have an adequate supply of blood. A hallmark of cancerous cells is their abnormal response to control mechanisms that regulate cell division in normal cells; thus, the cells continue to divide until they ultimately kill the host.

Angiogenesis is a highly regulated process under normal conditions, however many diseases are driven by persistent unregulated angiogenesis. Unregulated angiogenesis may either cause a particular disease directly or exacerbate an existing pathological condition. For example, ocular neovascularization has not only been implicated as the most common cause of blindness, but also is believed the dominant cause of many eye diseases. Further, in certain existing conditions, for example arthritis, newly formed capillary blood vessels invade the joints and destroy cartilage, or in the case of diabetes, new capillaries formed in the retina invade the vitreous, bleed, and cause blindness. Growth and metastasis of solid tumors are also dependent on angiogenesis (Folkman, J., Cancer Research, 46, 467-473 (1986), Folkman, J., Journal of the National Cancer Institute, 82, 4-6 (1989). It has been shown, for example, that tumors which enlarge to greater than 2 mm must obtain their own blood supply and do so by inducing the growth of new capillary blood vessels. Once these new blood vessels become embedded in the tumor, they provide a means for tumor cells to enter the circulation and metastasize to distant sites such as liver, lung or bone (Weidner, N., et al., The New England Journal of Medicine, 324(1), 1-8 (1991). Under conditions of unregulated angiogenesis, therapeutic methods designed to control, repress, and/or inhibit angiogenesis could lead to the abrogation or mitigation of these conditions and diseases.

Inflammation is related to a variety of disorders such as pain, headaches, fever, arthritis, asthma, bronchitis, menstrual cramps, tendonitis, bursitis, psoriasis, eczema, burns, dermatitis, inflammatory bowel syndrome, Crohn's disease, gastritis, irritable bowel syndrome, ulcerative colitis, vascular diseases, Hodgkin's disease, sclerodoma, rheumatic fever, type I diabetes, myasthenia gravis, sarcoidosis, nephrotic syndrome, Behcet's syndrome, polymyositis, hypersensitivity, conjunctivitis, gingivitis, post-injury swelling, myocardial ischemia, cerebral ischemia (stroke), sepsis and the like.

Heat-shock protein 90 (HSP-90) is a cellular chaperone protein required for the activation of several eukaryotic protein kinases, including the cyclin-dependent kinase CDK4. Geldanamycin, an inhibitor of the protein-refolding activity of HSP-90, has been shown to have antiproliferative and antitumor activities.

HSP-90 is a molecular chaperone that guides the normal folding, intracellular disposition and proteolytic turnover of many key regulators of cell growth and survival. Its function is subverted during oncogenesis to make malignant transformation possible and to facilitate rapid somatic evolution, and to allow mutant proteins to retain or even gain function. Inhibition of HSP-90 will slow those processes and thus has therapeutic use (Whitesell L, Lindquist, S L, Nature Rev. Cancer, 2005, 10, 761-72).

Ansamycin antibiotics, e.g., herbimycin A (HA), geldanamycin (GM), and 17-allylaminogeldanamycin (17-AAG) are thought to exert their anticancerous effects by tight binding of the N-terminus pocket of HSP-90, thereby destabilizing substrates that normally interact with HSP-90 (Stebbins, C. et al. Cell 1997, 89, 239-250). This pocket is highly conserved and has weak homology to the ATP-binding site of DNA gyrase (Stebbins, C. et al., supra; Grenert, J. P. et al. J. Biol. Chem. 1997, 272, 23843-50).

In vitro and in vivo studies have demonstrated that occupancy of this N-terminal pocket by ansamycins and other HSP-90 inhibitors alters HSP-90 function and inhibits protein folding. At high concentrations, ansamycins and other HSP-90 inhibitors have been shown to prevent binding of protein substrates to HSP-90 (Scheibel, T. H. et al. Proc. Natl. Acad. Sci. USA 1999, 96, 1297-302; Schulte, T. W. et al. J. Biol. Chem. 1995, 270, 24585-8 Whitesell, L., et al. Proc. Natl. Acad. Sci. USA 1994, 91, 8324-8328). Ansamycins have also been demonstrated to inhibit the ATP-dependent release of chaperone-associated protein substrates (Schneider, C. L. et al. Proc. Natl. Acad. Sci., USA 1996, 93, 14536-41; Sepp-Lorenzino et al. J. Biol. Chem. 1995, 270, 16580-16587). In either event, the substrates are degraded by a ubiquitin-dependent process in the proteasome (Schneider, C. L., supra; Sepp-Lorenzino, L., et al. J. Biol. Claim. 1995, 270, 16580-16587; Whitesell, L. et al. Proc. Natl. Acad. Sci. USA 1994, 91, 8324-8328). HSP-90 substrate destabilization occurs in tumor and non-transformed cells alike and has been shown to be especially effective on a subset of signaling regulators, e.g., Raf (Schulte, T. W. et al., Biochem. Biophys. Res. Commun. 1997, 239, 655-9 Schulte, T. W., et al., J. Biol. Chem. 1995, 270, 24585-8), nuclear steroid receptors (Segnitz, B.; U. Gehring J. Biol. Chem. 1997, 272, 18694-18701; Smith, D. F. et al. Mol. Cell. Biol. 1995, 15, 6804-12), v-Src (Whitesell, L., et al. Proc. Natl. Acad. Sci. USA 1994, 91, 8324-8328) and certain transmembrane tyrosine kinases (Sepp-Lorenzino, L. et al. J. Biol. Chez. 1995, 270, 16580-16587) such as EGF receptor (EGFR) and HER2/Neu (Hartmann, F., et al. Int. J. Cancer 1997, 70, 221-9; Miller, P. et al. Cancer Res. 1994, 54, 2724-2730; Mimnaugh, E. G., et al. J. Biol. Clzem. 1996, 271, 22796-801; Schnur, R. et al. J. Med. Chenu. 1995, 38, 3806-3812), CDK4, and mutant p53. Erlichman et al. Proc. AACR 2001, 42, abstract 4474. The ansamycin-induced loss of these proteins leads to the selective disruption of certain regulatory pathways and results in growth arrest at specific phases of the cell cycle (Muise-Heimericks, R. C. et al. J. Biol. Chez. 1998, 273, 29864-72), and apoptosis, and/or differentiation of cells so treated (Vasilevskaya, A. et al. Cancer Res., 1999, 59, 3935-40). Inhibitors of HSP-90 thus will be useful for the treatment and/or prevention of many types of cancers and proliferative disorders, and may also be useful as traditional antibiotics.

Inhibition of HSP-90 is also known to result in up regulation of the expression of the chaperone HSP70. HSP70 up regulation is considered to be of therapeutic benefit for treatment of a wide range of neurodegenerative diseases including, but not limited to: Alzheimer's disease; Parkinson's disease; Dementia with Lewy bodies; Amyotropic lateral scleriosis (ALS); Polyglutamine disease; Huntington's disease; Spinal and bulbar muscular atrophy (SBMA); and Spinocerebellar ataxias (SCA1-3,7). Therefore, the compounds described in the invention are of potential therapeutic use for treatment of such neurodegenerative diseases (Muchowski, P. J., Wacker J. L., Nat. Rev. Neurosci. 2005, 6, 11-22; Shen H. Y., et al. J. Biol. Chem. 2005, 280, 39962-9).

Inhibition of HSP-90 also has anti-fungal activity, both as a stand alone therapy and in combination with standard anti-fungal therapies such as the azole class of drugs. Therefore, the compounds described in the invention are of potential therapeutic use for treatment of fungal infections including, but not limited to, life threatening systemic fungal infections (Cowen, L. E., Lindquist, S., Science 2005, 309, 2185-9).

HSP-90 has also been shown to be important to viral transcription and replication, in particular for such processes in HIV-1 and Hepatitis C virus. See J Biol. Chem. 2000 Jan. 7; 275(1):279-87; J. Virol. 2004 December; 78(23):13122-31; and Biochem Biophys Res Commun. 2007 Feb. 23; 353(4):882-8. Epub 2006 Dec. 22. Inhibitors of HSP-90 have been shown to attenuate infection in animal models of polio infection. See Genes Dev. 2007 (21) 195-205.

Inhibitors of HSP-90 have been shown to attenuate inflammation via lowering the level of a number of client proteins associated inflammation process. See FASEB J. 2007 July; 21(9):2113-23.

Inhibition of HSP-90 is also expected to result in antimalarial activity; thus, inhibitors of this protein are useful as antimalarial drugs.

There is a continuing need for new methods of treating cancer, inflammation and inflammation-associated disorders, and conditions or diseases related to uncontrolled angiogenesis.

### SUMMARY OF THE INVENTION

In a broad aspect, the invention encompasses compounds of formula I shown below, pharmaceutical compositions containing those compounds and methods employing such compounds or compositions in the treatment of diseases and/or conditions related to cell proliferation, such as cancer, inflammation, arthritis, angiogenesis, or the like.

In a first aspect, the invention provides compounds of formula I, 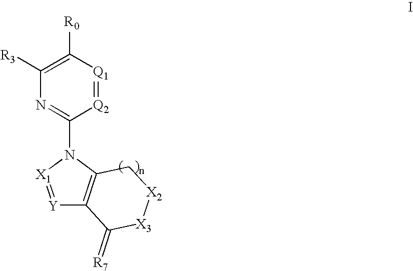
and pharmaceutically acceptable salts thereof, wherein R₀, R₃, R₇, Q₁, Q₂, X₁-X₃, n, and Y are defined herein.

The invention also provides intermediates that are useful in making the compounds of formula I.

The invention also provides pharmaceutical compositions comprising a compound or pharmaceutically acceptable salt of Formula I and at least one pharmaceutically acceptable carrier, solvent, adjuvant or diluent.

The invention further provides methods of treating disease such as cancer, inflammation, arthritis, angiogenesis, and infection in a patient in need of such treatment, comprising administering to the patient a compound or pharmaceutically acceptable salt of Formula I, or a pharmaceutical composition comprising a compound or salt of Formula I.

The invention also provides the use of a compound or salt according to Formula I for the manufacture of a medicament for use in treating cancer, inflammation, arthritis, angiogenesis, or infection.

The invention also provides methods of preparing the compounds of the invention and intermediates used in those methods.

The invention also provides methods of treating a disease or condition related to cell proliferation comprising administering a therapeutically effective amount of a compound or salt of Formula I to a patient in need of such treatment.

The invention also provides methods of treating a disease or condition related to cell proliferation comprising administering a therapeutically effective amount of a compound or salt of Formula I to a patient in need of such treatment, where the disease of condition is cancer, inflammation, or arthritis.

The invention further provides methods of treating a subject suffering from a disease or disorder of proteins that are either client proteins for HSP-90 or indirectly affect its client proteins, comprising administering to a subject in need of such treatment a therapeutically effective amount of a compound or salt of Formula I.

The invention further provides methods of treating a subject suffering from a disease or disorder of proteins that are either client proteins for HSP-90 or indirectly affect its client proteins, comprising administering to a subject in need of such treatment a therapeutically effective amount of a compound or salt of Formula I, wherein the HSP-90 mediated disorder is selected from the group of inflammatory diseases, infections, autoimmune disorders, stroke, ischemia, cardiac disorders, neurological disorders, fibrogenetic disorders, proliferative disorders, tumors, leukemias, neoplasms, cancers, carcinomas, metabolic diseases and malignant disease.

The invention further provides methods of treating a subject suffering from a fibrogenetic disorder of proteins that are either client proteins for HSP-90 or indirectly affect its client proteins, comprising administering to a subject in need of such treatment a therapeutically effective amount of a compound or salt of Formula I, wherein the fibrogenetic disorder is selected from the group of scleroderma, polymyositis, systemic lupus, rheumatoid arthritis, liver cirrhosis, keloid formation, interstitial nephritis and pulmonary fibrosis.

The invention provides methods of protecting a subject from infection caused by an organism selected from Plasmodium species, preferably Plasmodium falciparum. These methods comprise administering a compound or salt of Formula I, preferably in an effective amount, to a subject at risk of infection due to exposure to such organism.

The invention additionally provides methods of reducing the level of infection in a subject where the infection is caused by an organism selected from Plasmodium species, again preferably Plasmodium falciparum. These methods comprise administering to an infected subject an effective amount of a compound or salt of Formula I.

The invention further provides methods for treating a patient infected with a metazoan parasite. These methods involve administering an amount of a compound of formula I effective to kill the parasite.

The invention further provides methods for treating a patient infected with a metazoan parasite wherein the parasite is Plasmodium falciparum. These methods involve administering an amount of a compound or salt of the invention effective to kill the parasite.

The invention also provides methods of treating and/or preventing viral infections in patients in need of such treatment comprising administration of a compound or salt of formula I.

The invention further encompasses kits comprising compounds of the invention or a pharmaceutical composition thereof in a package with instructions for using the compound or composition.

In another aspect, the invention provides combination therapy, i.e., treatment of a patient in need thereof with a combination of a compound of formula I with other drugs or therapies known to be effective to treat the disease to enhance overall effectiveness of therapy. The combination may be in a single dosage form, e.g., a single tablet, or may involve simultaneous or sequential administration of two or more different dosage forms, e.g., an HSP-90 inhibitor of the invention, intravenous chemotherapy administration, and radiation therapy.

The invention further provides methods for treating a fungal infection in a patient in need of such treatment, comprising administering an effective amount of a compound or salt of Formula I and an optional anti-fungal agent or drug.

### DETAILED DESCRIPTION OF THE INVENTION

In a first aspect, the invention provides compounds of formula I, 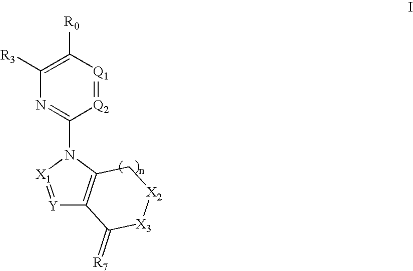
and pharmaceutically acceptable salts thereof, wherein
Q₁ and Q₂ are independently N or CR₁, wherein one and only one of Q₁ and Q₂ must be N;
R₁ and R₀ are independently hydrogen, halogen, hydroxyl, cyano, nitro, amino, mono- or di-(C₁-C₁₀)alkylamino, carboxamido, —NHaryl, —NHheteroaryl, C₁-C₁₀ haloalkyl, C₁-C₁₀ alkyl, C₃-C₁₀ cycloalkyl, C₃-C₁₀ heterocycloalkyl, aryl, heteroaryl, or a group of the formula 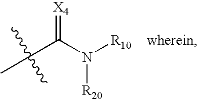
X₄ is O, NH, NOH, or S; and
R₁₀ and R₂₀ are independently H, OH, C₁-C₁₀ haloalkyl, C₁-C₁₀alkyl, C₃-C₁₀cycloalkyl, C₃-C₁₀ heterocycloalkyl, aryl, or heteroaryl;
   wherein each alkyl, aryl, cycloalkyl, heterocycloalkyl, and heteroaryl group is optionally substituted with one to four groups which are each independently C₁-C₆ alkyl, C₁-C₆ alkoxy, halogen, carboxy, oxo, amino, mono- or di-(C₁-C₆)alkylamino, cyano, nitro, halo(C₁-C₆)alkyl, halo(C₁-C₆)alkoxy, carboxamide, heterocycloalkyl, aryl, or heteroaryl, wherein
the aryl and heteroaryl groups are optionally substituted with from one to four groups what are each independently C₁-C₆ alkyl, C₁-C₆ alkoxy, halogen, hydroxy, amino, mono- or di-(C₁-C₆)alkylamino, halo(C₁-C₆)alkyl, or carboxamide;
   R₃ is (a) H; (b) halo; or
(c) a C₁-C₁₅ alkyl group where up to six of the carbon atoms in said alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other, wherein
   R₂₂ is
      (i) heteroaryl,
      (ii) aryl,
      (iii) saturated or unsaturated C₃-C₁₀ cycloalkyl, or
      (iv) saturated or unsaturated C₃-C₁₀ heterocycloalkyl, wherein
      each aryl, heteroaryl, saturated or unsaturated cycloalkyl, or saturated or unsaturated heterocycloalkyl, independently, is optionally substituted with at least one group, which independently is hydroxy, halo, amino, cyano, carboxy, carboxamido, nitro, oxo, —S—(C₁-C₆)alkyl, —SO₂—(C₁-C₆)alkyl, —SO₂-aryl, —SO—(C₁-C₆)alkyl, —SO-aryl, —SO₂NH₂, —SO₂NH—(C₁-C₆)alkyl, —SO₂NH-aryl, (C₁-C₆)alkoxy, or mono- or di-(C₁-C₁₀)alkylamino; and
   each R₂₂ is optionally fused to a C₆-C₁₀ aryl group, C₅-C₈ saturated cyclic group, or a C₅-C₁₀ heterocycloalkyl group;
wherein each (c) is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S—(C₁-C₆)alkyl, —SO₂—(C₁-C₆)alkyl, —SO₂NH₂, —SO₂NH—(C₁-C₆)alkyl, —SO₂NH-aryl, —SO₂-aryl, —SO—(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, or R₂₃, wherein
   R₂₃ is
      (1) heteroaryl,
      (2) aryl,
      (3) saturated or unsaturated C₅-C₁₀ cycloalkyl, or
      (4) saturated or unsaturated C₅-C₁₀ heterocycloalkyl, and
   the R₂₃ groups are optionally substituted at least one group which is independently hydroxy, oxo, halo, amino, cyano, nitro, —SH, —S—(C₁-C₆)alkyl, —SO₂—(C₁-C₆)alkyl, —SO₂-aryl, —SO—(C₁-C₆)alkyl, —SO-aryl, —SO₂NH₂, —SO₂NH—(C₁-C₆)alkyl, —SO₂NH-aryl, (C₁-C₆)alkoxy, or mono- or di-(C₁-C₁₀)alkylamino;
      R₇ is O, S, or NR_{7′}, wherein
R_{7′} is H, —OH, —NH₂, —NHR₂₂, —NH—(C₁-C₆ alkyl), —O—(C₀-C₆)alkyl-R₂₂, or —(C₁-C₆ alkoxy optionally substituted with carboxy);
   X₁ is N or CR_{C};
each R_{C} independently is hydrogen, halogen, cyano, nitro, C₁-C₁₀ alkyl, C₂-C₁₀ alkenyl, C₂-C₁₀ alkynyl, C₁-C₁₀ haloalkyl, C₃-C₇ cycloalkyl, C₃-C₇ cycloalkyl(C₁-C₁₀)alkyl, heterocycloalkyl, aryl, or heteroaryl, wherein
   each alkyl, aryl, cycloalkyl, heterocycloalkyl, and heteroaryl group is optionally substituted with from 1-4 groups that are independently C₁-C₆ alkyl, C₁-C₆ alkoxy, halogen, hydroxy, amino, mono- or di-(C₁-C₆) alkylamino, cyano, nitro, halo(C₁-C₆)alkyl, halo(C₁-C₆)alkoxy, carboxamide, heterocycloalkyl, aryl, or heteroaryl, wherein
      the aryl and heteroaryl groups are optionally substituted with from 1-4 groups that are independently C₁-C₆ alkyl, C₁-C₆ alkoxy, halogen, hydroxy, amino, mono- or di-(C₁-C₆)alkylamino, halo(C₁-C₆)alkyl, or carboxamide;
         Y is N or CR_{C};
         X₂ and X₃ are independently C(R₅)(R₆), O, N(R₅), or S(O)ₚ wherein
each R₅ and R₆ is independently hydrogen, C₁-C₆ alkyl, or mono- or di-(C₁-C₆)alkylamino(C₁-C₆)alkyl
or R₅ and R₆ together with the carbon to which they are attached form a 3-8 membered cycloalkyl or heterocycloalkyl ring; and
p is 0, 1, or 2; and
   n is 0, 1, 2, 3, or 4.

Preferred compounds of formula I include those where R₇ is O or N—OH. More preferred compounds of formula I are those wherein R₇ is O.

Other preferred compounds of formula I are those where n is 0, 1, or 2. More preferred compounds of formula I are those wherein n is 1.

Other preferred compounds of formula I are those where R₀ is cyano, hydroxyl, nitro, amino, mono- or di-(C₁-C₁₀) alkylamino, or a group of the formula 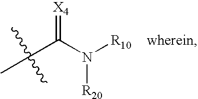
X₄ is O, NH, NOH, or S; and
R₁₀ and R₂₀ are independently H, OH, C₁-C₁₀ haloalkyl, or C₁-C₁₀alkyl.

More preferred compounds of formula I are those wherein R₀ is cyano or —CONH₂. More preferred compounds of formula I are those wherein R₀ is cyano. More preferred compounds of formula I are those wherein R₀ is —CONH₂.

Preferred compounds of Formula I include those where R₃ is hydrogen, halo, or -Z₁R_{Z1}, wherein Z₁ is —O—, —NH—, —S(O)ₚ—, or —S(O)₂NH—, wherein p is 0, 1 or 2; and R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S—(C₁-C₆)alkyl, —SO₂—(C₁-C₆)alkyl, —SO₂NH₂, —SO₂NH—(C₁-C₆)alkyl, —SO₂NH-aryl, —SO₂-aryl, —SO—(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, or R₂₃.

Even more preferred compounds of Formula I include those where R₃ is hydrogen, halo, or -Z₁R_{Z1}, wherein Z₁ is —O— or —NH—; and R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S—(C₁-C₆)alkyl, —SO₂—(C₁-C₆)alkyl, —SO₂NH₂, —SO₂NH—(C₁-C₆)alkyl, —SO₂NH-aryl, —SO₂-aryl, —SO—(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, or R₂₃.

Additional preferred compounds of Formula I include those where R₃ is hydrogen, halo, or —N(H)R_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S—(C₁-C₆)alkyl, —SO₂—(C₁-C₆)alkyl, —SO₂NH₂, —SO₂NH—(C₁-C₆)alkyl, —SO₂NH-aryl, —SO₂-aryl, —SO—(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, or R₂₃.

Most preferred compounds of Formula I include those where R₃ is hydrogen, halo, or —N(H)R_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, or R₂₃.

Additional preferred compounds of Formula I include those where R₃ is hydrogen, halo, or —OR_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S—(C₁-C₆)alkyl, —SO₂—(C₁-C₆)alkyl, —SO₂NH₂, —SO₂NH—(C₁-C₆)alkyl, —SO₂NH-aryl, —SO₂-aryl, —SO—(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, or R₂₃.

Most preferred compounds of Formula I include those where R₃ is hydrogen, halo, or —OR_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, or R₂₃.

Other preferred compounds of formula I are those where X₁ is N.

Other preferred compounds of formula I are those where Y is N.

Other preferred compounds of formula I are those where X₁ is CR_{C}.

Other preferred compounds of formula I are those where Y is CR_{C}.

More preferred embodiments of formula I are those compounds where X₁ is N and Y is CR_{C}. Even more preferred compounds of formula I are those where, X₁ is N and Y is CR_{C}, wherein R_{C} is hydrogen, halogen, C₁-C₁₀ alkyl, C₁-C₁₀ haloalkyl, C₃-C₇ cycloalkyl, or C₃-C₇ cycloalkyl(C₁-C₁₀)alkyl, wherein
each alkyl or cycloalkyl is optionally substituted with from 1-4 groups that are independently C₁-C₆ alkyl, C₁-C₆ alkoxy, halogen, hydroxy, amino, mono- or di-(C₁-C₆)alkylamino, cyano, nitro, halo(C₁-C₆)alkyl, halo(C₁-C₆)alkoxy, carboxamide, heterocycloalkyl, aryl, or heteroaryl, wherein
   the aryl and heteroaryl groups are optionally substituted with from 1-4 groups that are independently C₁-C₆ alkyl, C₁-C₆ alkoxy, halogen, hydroxy, amino, mono- or di-(C₁-C₆)alkylamino, halo(C₁-C₆)alkyl, or carboxamide.
      Even more preferred compounds of formula I are those where, X₁ is N and Y is CR_{C}, wherein R_{C} is hydrogen, halogen, C₁-C₁₀ alkyl, C₁-C₁₀ haloalkyl, C₃-C₇ cycloalkyl, or C₃-C₇ cycloalkyl(C₁-C₁₀)alkyl.

Even more preferred compounds of formula I are those where, X₁ is N and Y is CR_{C}, wherein R_{C} is hydrogen, halogen, C₁-C₃ alkyl, C₁-C₃ haloalkyl, cyclopropyl, or cyclopropylmethyl.

Even more preferred compounds of formula I are those where, X₁ is N and Y is CR_{C}, wherein R_{C} is independently hydrogen, halogen, methyl, ethyl, fluoromethyl, difluoromethyl, trifluoromethyl, cyclopropyl, or cyclopropylmethyl.

In another embodiment, more preferred compounds formula I are those where X₁ and Y are each CR_{C}, wherein each R_{C} is independently hydrogen, halogen, C₁-C₁₀ alkyl, C₁-C₁₀ haloalkyl, C₃-C₇ cycloalkyl, or C₃-C₇ cycloalkyl(C₁-C₁₀)alkyl, wherein
each alkyl or cycloalkyl is optionally substituted with from 1-4 groups that are independently C₁-C₆ alkyl, C₁-C₆ alkoxy, halogen, hydroxy, amino, mono- or di-(C₁-C₆)alkylamino, cyano, nitro, halo(C₁-C₆)alkyl, halo(C₁-C₆)alkoxy, carboxamide, heterocycloalkyl, aryl, or heteroaryl, wherein
   the aryl and heteroaryl groups are optionally substituted with from 1-4 groups that are independently C₁-C₆ alkyl, C₁-C₆ alkoxy, halogen, hydroxy, amino, mono- or di-(C₁-C₆)alkylamino, halo(C₁-C₆)alkyl, or carboxamide.
      Even more preferred compounds of formula I are those where, X₁ and Y are each CR_{C}, wherein each R_{C} is independently hydrogen, halogen, C₁-C₁₀ alkyl, C₁-C₁₀ haloalkyl, C₃-C₇ cycloalkyl, or C₃-C₇ cycloalkyl(C₁-C₁₀)alkyl.

Even more preferred compounds of formula I are those where, X₁ and Y are each CR_{C}, wherein each R_{C} is independently hydrogen, halogen, C₁-C₃ alkyl, C₁-C₃ haloalkyl, cyclopropyl, or cyclopropylmethyl.

Even more preferred compounds of formula I are those where, X₁ and Y are each CR_{C}, wherein each R_{C} is independently hydrogen, halogen, methyl, ethyl, fluoromethyl, difluoromethyl, trifluoromethyl, cyclopropyl, or cyclopropylmethyl.

In another embodiment, preferred compounds of formula I are those where X₃ is CH₂.

In another embodiment, preferred compounds of formula I are those where X₂ is CR₅R₆.

In a preferred embodiment, the invention provides compounds of formula I where X₃ is CH₂ and X₂ is CR₅R₆.

In a more preferred embodiment, the invention provides compounds of formula I where X₃ is CH₂ and X₂ is CR₅R₆, wherein R₅ and R₆ are each independently hydrogen or C₁-C₆ alkyl.

In a more preferred embodiment, the invention provides compounds of formula I where X₃ is CH₂ and X₂ is CR₅R₆, wherein R₅ and R₆ are each independently hydrogen or C₁-C₃ alkyl.

Other preferred compounds of formula I are those where Q₁ is N and Q₂ is CR₁.

Other preferred compounds of formula I are those where Q₁ is N and Q₂ is CR₁, wherein R₁ is hydrogen, halogen, amino, C₁-C₁₀ alkyl, or C₁-C₁₀ haloalkyl.

Other preferred compounds of formula I are those where Q₂ is N and Q₁ is CR₁.

Other preferred compounds of formula I are those where Q₂ is N and Q₁ is CR₁, wherein R₁ is hydrogen, halogen, amino, C₁-C₁₀ alkyl, or C₁-C₁₀ haloalkyl.

In compounds of formula I where R₀ is the group 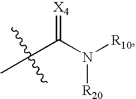
and R₁₀ and/or R₂₀ is haloalkyl, preferred haloalkyl groups are difluoromethyl or fluoromethyl, or C₂-C₆ haloalkyl groups where the carbon atom attached to the nitrogen is not substituted with halogen.

In another embodiment, the invention provides compounds according to formula (IIa) and (IIb), 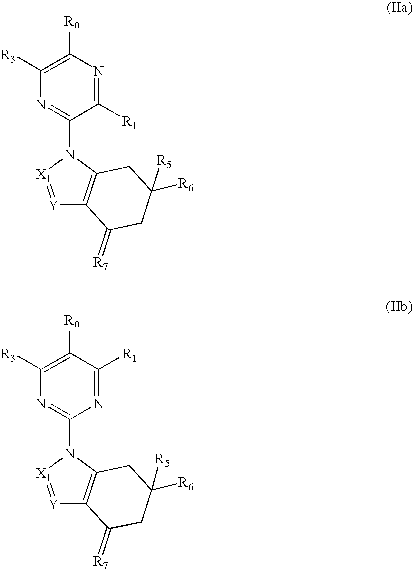
wherein R₀, R₁, R₃, R₅, R₆, R₇, X₁, and Y are as defined for formula (I).

Preferred compounds of formula IIa and IIb include those where R₇ is O or N—OH. More preferred compounds of formula IIa and IIb are those wherein R₇ is O.

Other preferred compounds of formula IIa and IIb are those where R₁ is hydrogen, halogen, amino, C₁-C₁₀ alkyl, or C₁-C₁₀ haloalkyl.

Other preferred compounds of formula IIa and IIb are those where R₀ is cyano, hydroxyl, nitro, amino, mono- or di-(C₁-C₁₀)alkylamino, or a group of the formula 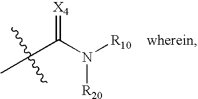
X₄ is O, NH, NOH, or S; and
R₁₀ and R₂₀ are independently H, OH, C₁-C₁₀ haloalkyl, or C₁-C₁₀alkyl.

More preferred compounds of formula IIa and IIb are those wherein R₀ is cyano or —CONH₂. More preferred compounds of formula IIa and IIb are those wherein R₀ is cyano. More preferred compounds of formula IIa and IIb are those wherein R₀ is —CONH₂.

Preferred compounds of Formula IIa and IIb include those where R₃ is hydrogen, halo, or -Z₁R_{Z1}, wherein Z₁ is —O—, —NH—, —S(O)ₚ—, or —S(O)₂NH—, wherein p is 0, 1 or 2; and R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S—(C₁-C₆)alkyl, —SO₂—(C₁-C₆)alkyl, —SO₂NH₂, —SO₂NH—(C₁-C₆)alkyl, —SO₂NH-aryl, —SO₂-aryl, —SO—(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, or R₂₃.

Even more preferred compounds of Formula IIa and IIb include those where R₃ is hydrogen, halo, or -Z₁R_{Z1}, wherein Z₁ is —O— or —NH—; and R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S—(C₁-C₆)alkyl, —SO₂—(C₁-C₆)alkyl, —SO₂NH₂, —SO₂NH—(C₁-C₆)alkyl, —SO₂NH-aryl, —SO₂-aryl, —SO—(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, or R₂₃.

Additional preferred compounds of Formula IIa and IIb include those where R₃ is hydrogen, halo, or —N(H)R_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S—(C₁-C₆)alkyl, —SO₂—(C₁-C₆)alkyl, —SO₂NH₂, —SO₂NH—(C₁-C₆)alkyl, —SO₂NH-aryl, —SO₂-aryl, —SO—(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, or R₂₃.

Most preferred compounds of Formula IIa and IIb include those where R₃ is hydrogen, halo, or —N(H)R_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, or R₂₃.

Additional preferred compounds of Formula IIa and IIb include those where R₃ is hydrogen, halo, or —OR_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S—(C₁-C₆)alkyl, —SO₂—(C₁-C₆)alkyl, —SO₂NH₂, —SO₂NH—(C₁-C₆)alkyl, —SO₂NH-aryl, —SO₂-aryl, —SO—(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, or R₂₃.

Most preferred compounds of Formula IIa and IIb include those where R₃ is hydrogen, halo, or —OR_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, or R₂₃.

Other preferred compounds of formula IIa and IIb are those where X₁ is N.

Other preferred compounds of formula IIa and IIb are those where Y is N.

Other preferred compounds of formula IIa and IIb are those where X₁ is CR_{C}.

Other preferred compounds of formula IIa and IIb are those where Y is CR_{C}.

More preferred embodiments of formula IIa and IIb are those compounds where X₁ is N and Y is CR_{C}. Even more preferred compounds of formula IIa and IIb are those where, X₁ is N and Y is CR_{C}, wherein R_{C} is hydrogen, halogen, C₁-C₁₀ alkyl, C₁-C₁₀ haloalkyl, C₃-C₇ cycloalkyl, or C₃-C₇ cycloalkyl(C₁-C₁₀)alkyl, wherein
each alkyl or cycloalkyl is optionally substituted with from 1-4 groups that are independently C₁-C₆ alkyl, C₁-C₆ alkoxy, halogen, hydroxy, amino, mono- or di-(C₁-C₆)alkylamino, cyano, nitro, halo(C₁-C₆)alkyl, halo(C₁-C₆)alkoxy, carboxamide, heterocycloalkyl, aryl, or heteroaryl, wherein
   the aryl and heteroaryl groups are optionally substituted with from 1-4 groups that are independently C₁-C₆ alkyl, C₁-C₆ alkoxy, halogen, hydroxy, amino, mono- or di-(C₁-C₆)alkylamino, halo(C₁-C₆)alkyl, or carboxamide.
      Even more preferred compounds of formula IIa and IIb are those where, X₁ is N and Y is CR_{C}, wherein R_{C} is hydrogen, halogen, C₁-C₁₀ alkyl, C₁-C₁₀ haloalkyl, C₃-C₇ cycloalkyl, or C₃-C₇ cycloalkyl(C₁-C₁₀)alkyl.

Even more preferred compounds of formula IIa and IIb are those where, X₁ is N and Y is CR_{C}, wherein R_{C} is hydrogen, halogen, C₁-C₃ alkyl, C₁-C₃ haloalkyl, cycloopropyl, or cyclopropylmethyl.

Even more preferred compounds of formula IIa and IIb are those where, X₁ is N and Y is CR_{C}, wherein R_{C} is independently hydrogen, halogen, methyl, ethyl, fluoromethyl, difluoromethyl, trifluoromethyl, cyclopropyl, or cyclopropylmethyl.

In another embodiment, more preferred compounds formula IIa and IIb are those where X₁ and Y are each CR_{C}, wherein each R_{C} is independently hydrogen, halogen, C₁-C₁₀ alkyl, C₁-C₁₀ haloalkyl, C₃-C₇ cycloalkyl, or C₃-C₇ cycloalkyl(C₁-C₁₀)alkyl, wherein
each alkyl or cycloalkyl is optionally substituted with from 1-4 groups that are independently C₁-C₆ alkyl, C₁-C₆ alkoxy, halogen, hydroxy, amino, mono- or di-(C₁-C₆)alkylamino, cyano, nitro, halo(C₁-C₆)alkyl, halo(C₁-C₆)alkoxy, carboxamide, heterocycloalkyl, aryl, or heteroaryl, wherein
   the aryl and heteroaryl groups are optionally substituted with from 1-4 groups that are independently C₁-C₆ alkyl, C₁-C₆ alkoxy, halogen, hydroxy, amino, mono- or di-(C₁-C₆)alkylamino, halo(C₁-C₆)alkyl, or carboxamide.
      Even more preferred compounds of formula IIa and IIb are those where, X₁ and Y are each CR_{C}, wherein each R_{C} is independently hydrogen, halogen, C₁-C₁₀ alkyl, C₁-C₁₀ haloalkyl, C₃-C₇ cycloalkyl, or C₃-C₇ cycloalkyl(C₁-C₁₀)alkyl.

Even more preferred compounds of formula IIa and IIb are those where, X₁ and Y are each CR_{C}, wherein each R_{C} is independently hydrogen, halogen, C₁-C₃ alkyl, C₁-C₃ haloalkyl, cyclopropyl, or cyclopropylmethyl.

Even more preferred compounds of formula IIa and IIb are those where, X₁ and Y are each CR_{C}, wherein each R_{C} is independently hydrogen, halogen, methyl, ethyl, fluoromethyl, difluoromethyl, trifluoromethyl, cyclopropyl, or cyclopropylmethyl.

In another embodiment, the invention provides compounds according to formula (IIIa) and (IIIb), 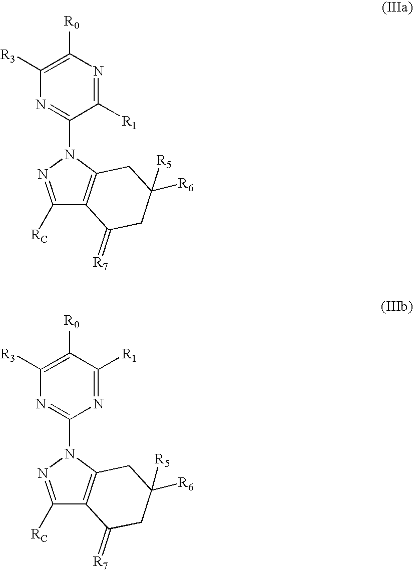
wherein R₀, R₁, R₃, R₅, R₆, R₇, and R_{C} are as defined for formula (I).

Preferred compounds of formula IIIa and IIIb include those where R₇ is O or N—OH. More preferred compounds of formula IIIa and IIIb are those wherein R₇ is O.

Other preferred compounds of formula IIIa and IIIb are those where R₁ is hydrogen, halogen, amino, C₁-C₁₀ alkyl, or C₁-C₁₀ haloalkyl.

Other preferred compounds of formula IIIa and IIIb are those where R₀ is cyano, hydroxyl, nitro, amino, mono- or di-(C₁-C₁₀) alkylamino, or a group of the formula 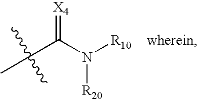
X₄ is O, NH, NOH, or S; and
R₁₀ and R₂₀ are independently H, OH, C₁-C₁₀ haloalkyl, or C₁-C₁₀alkyl.

More preferred compounds of formula IIIa and IIIb are those wherein R₀ is cyano or —CONH₂. More preferred compounds of formula IIIa and IIIb are those wherein R₀ is cyano. More preferred compounds of formula IIIa and IIIb are those wherein R₀ is —CONH₂.

Preferred compounds of Formula IIIa and IIIB include those where R₃ is hydrogen, halo, or -Z₁R_{Z1}, wherein Z₁ is —O—, —NH—, —S(O)ₚ—, or —S(O)₂NH—, wherein p is 0, 1 or 2; and R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S—(C₁-C₆)alkyl, —SO₂—(C₁-C₆)alkyl, —SO₂NH₂, —SO₂NH—(C₁-C₆)alkyl, —SO₂NH-aryl, —SO₂-aryl, —SO—(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, or R₂₃.

Even more preferred compounds of Formula IIIa and IIIB include those where R₃ is hydrogen, halo, or -Z₁R_{Z1}, wherein Z₁ is —O— or —NH—; and R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S—(C₁-C₆)alkyl, —SO₂—(C₁-C₆)alkyl, —SO₂NH₂, —SO₂NH—(C₁-C₆)alkyl, —SO₂NH-aryl, —SO₂-aryl, —SO—(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, or R₂₃.

Additional preferred compounds of Formula IIIa and IIIb include those where R₃ is hydrogen, halo, or —N(H)R_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S—(C₁-C₆)alkyl, —SO₂—(C₁-C₆)alkyl, —SO₂NH₂, —SO₂NH—(C₁-C₆)alkyl, —SO₂NH-aryl, —SO₂-aryl, —SO—(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, or R₂₃.

Most preferred compounds of Formula IIIa and IIIb include those where R₃ is hydrogen, halo, or —N(H)R_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, or R₂₃.

Additional preferred compounds of Formula IIIa and IIIb include those where R₃ is hydrogen, halo, or —OR_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S—(C₁-C₆)alkyl, —SO₂—(C₁-C₆)alkyl, —SO₂NH₂, —SO₂NH—(C₁-C₆)alkyl, —SO₂NH-aryl, —SO₂-aryl, —SO—(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, or R₂₃.

Most preferred compounds of Formula IIIa and IIIb include those where R₃ is hydrogen, halo, or —OR_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, or R₂₃.

Even more preferred compounds of formula IIIa and IIIb are those where R_{C} is hydrogen, halogen, C₁-C₁₀ alkyl, C₁-C₁₀ haloalkyl, C₃-C₇ cycloalkyl, or C₃-C₇ cycloalkyl(C₁-C₁₀)alkyl.

Even more preferred compounds of formula IIIa and IIIb are those where R_{C} is hydrogen, halogen, C₁-C₃ alkyl, C₁-C₃ haloalkyl, cycloopropyl, or cyclopropylmethyl.

Even more preferred compounds of formula IIIa and IIIb are those where R_{C} is independently hydrogen, halogen, methyl, ethyl, fluoromethyl, difluoromethyl, trifluoromethyl, cyclopropyl, or cyclopropylmethyl.

In another embodiment, the invention provides compounds according to formula (IVa) and (IVb), 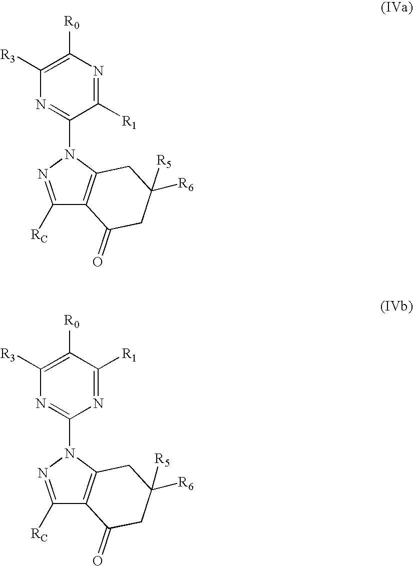
wherein R₀, R₁, R₃, R₅, R₆, and R_{C} are as defined for formula (I).

Preferred compounds of formula IVa and IVb are those where R₁ is hydrogen, halogen, amino, C₁-C₁₀ alkyl, or C₁-C₁₀ haloalkyl.

Other preferred compounds of formula IVa and IVb are those where R₀ is cyano, hydroxyl, nitro, amino, mono- or di-(C₁-C₁₀)alkylamino, or a group of the formula 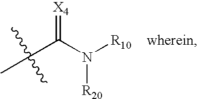
X₄ is O, NH, NOH, or S; and
R₁₀ and R₂₀ are independently H, OH, C₁-C₁₀ haloalkyl, or C₁-C₁₀alkyl.

More preferred compounds of formula IVa and IVb are those wherein R₀ is cyano or —CONH₂. More preferred compounds of formula IVa and IVb are those wherein R₀ is cyano. More preferred compounds of formula IVa and IVb are those wherein R₀ is —CONH₂.

Preferred compounds of Formula IVa and IVb include those where R₃ is hydrogen, halo, or -Z₁R_{Z1}, wherein Z₁ is —O—, —NH—, —S(O)ₚ—, or —S(O)₂NH—, wherein p is 0, 1 or 2; and R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S—(C₁-C₆)alkyl, —SO₂—(C₁-C₆)alkyl, —SO₂NH₂, —SO₂NH—(C₁-C₆)alkyl, —SO₂NH-aryl, —SO₂-aryl, —SO—(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, or R₂₃.

Even more preferred compounds of Formula IVa and IVb include those where R₃ is hydrogen, halo, or -Z₁R_{Z1}, wherein Z₁ is —O— or —NH—; and R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S—(C₁-C₆)alkyl, —SO₂—(C₁-C₆)alkyl, —SO₂NH₂, —SO₂NH—(C₁-C₆)alkyl, —SO₂NH-aryl, —SO₂-aryl, —SO—(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, or R₂₃.

Additional preferred compounds of Formula IVa and IVb include those where R₃ is hydrogen, halo, or —N(H)R_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S—(C₁-C₆)alkyl, —SO₂—(C₁-C₆)alkyl, —SO₂NH₂, —SO₂NH—(C₁-C₆)alkyl, —SO₂NH-aryl, —SO₂-aryl, —SO—(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, or R₂₃.

Most preferred compounds of Formula IVa and IVb include those where R₃ is hydrogen, halo, or —N(H)R_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, or R₂₃.

Additional preferred compounds of Formula IVa and IVb include those where R₃ is hydrogen, halo, or —OR_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S—(C₁-C₆)alkyl, —SO₂—(C₁-C₆)alkyl, —SO₂NH₂, —SO₂NH—(C₁-C₆)alkyl, —SO₂NH-aryl, —SO₂-aryl, —SO—(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, or R₂₃.

Most preferred compounds of Formula IVa and IVb include those where R₃ is hydrogen, halo, or —OR_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, or R₂₃.

Even more preferred compounds of formula IVa and IVb are those where R_{C} is hydrogen, halogen, C₁-C₁₀ alkyl, C₁-C₁₀ haloalkyl, C₃-C₇ cycloalkyl, or C₃-C₇ cycloalkyl(C₁-C₁₀)alkyl.

Even more preferred compounds of formula IVa and IVb are those where R_{C} is hydrogen, halogen, C₁-C₃ alkyl, C₁-C₃ haloalkyl, cycloopropyl, or cyclopropylmethyl.

Even more preferred compounds of formula IVa and IVb are those where R_{C} is independently hydrogen, halogen, methyl, ethyl, fluoromethyl, difluoromethyl, trifluoromethyl, cyclopropyl, or cyclopropylmethyl.

In another embodiment, the invention provides compounds according to formula (Va) and (Vb), 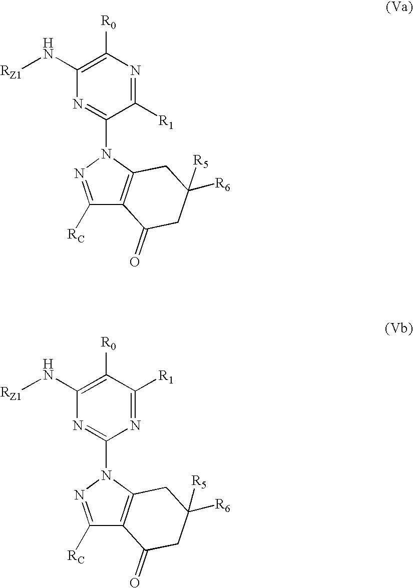
wherein R₀, R₁, R₅, R₆, R_{C}, and R_{Z1} are as defined for formula (I).

Preferred compounds of formula Va and Vb are those where R₁ is hydrogen, halogen, amino, C₁-C₁₀ alkyl, or C₁-C₁₀ haloalkyl.

Other preferred compounds of formula Va and Vb are those where R₀ is cyano, hydroxyl, nitro, amino, mono- or di-(C₁-C₁₀)alkylamino, or a group of the formula 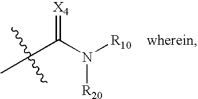
X₄ is O, NH, NOH, or S; and
R₁₀ and R₂₀ are independently H, OH, C₁-C₁₀ haloalkyl, or C₁-C₁₀alkyl.

More preferred compounds of formula Va and Vb are those wherein R₀ is cyano or —CONH₂. More preferred compounds of formula Va and Vb are those wherein R₀ is cyano. More preferred compounds of formula Va and Vb are those wherein R₀ is —CONH₂.

Preferred compounds of Formula Va and Vb include those where R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, or R₂₃.

More preferred compounds of Formula Va and Vb include those where R_{Z1} is a C₁-C₁₄ alkyl group where up to five of the carbon atoms in the alkyl group are optionally replaced independently by R₂₂, carbonyl, N, or O, with the proviso that two O atoms are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, or R₂₃.

Even more preferred compounds of formula Va and Vb are those where R_{C} is hydrogen, halogen, C₁-C₁₀ alkyl, C₁-C₁₀ haloalkyl, C₃-C₇ cycloalkyl, or C₃-C₇ cycloalkyl(C₁-C₁₀)alkyl.

Even more preferred compounds of formula Va and Vb are those where R_{C} is hydrogen, halogen, C₁-C₃ alkyl, C₁-C₃ haloalkyl, cycloopropyl, or cyclopropylmethyl.

Even more preferred compounds of formula Va and Vb are those where R_{C} is independently hydrogen, halogen, methyl, ethyl, fluoromethyl, difluoromethyl, trifluoromethyl, cyclopropyl, or cyclopropylmethyl.

In another embodiment, the invention provides compounds according to formula (VIa) and (VIb), 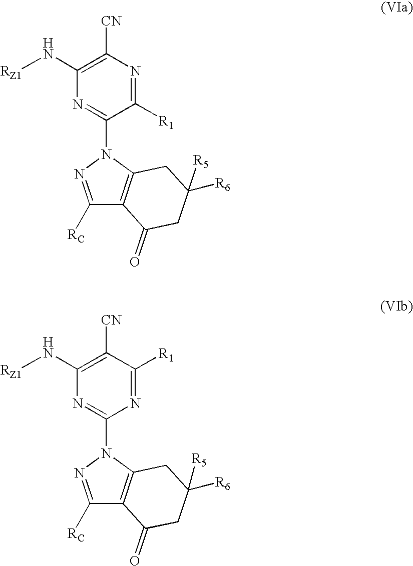
wherein R₁, R₅, R₆, R_{C}, and R_{Z1} are as defined for formula (I).

Preferred compounds of formula VIa and VIb are those where R₁ is hydrogen, halogen, amino, C₁-C₁₀ alkyl, or C₁-C₁₀ haloalkyl.

Preferred compounds of Formula VIa and VIb include those where R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, or R₂₃.

More preferred compounds of Formula VIa and VIb include those where R_{Z1} is a C₁-C₁₄ alkyl group where up to five of the carbon atoms in the alkyl group are optionally replaced independently by R₂₂, carbonyl, N, or O, with the proviso that two O atoms are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, or R₂₃.

Even more preferred compounds of formula VIa and VIb are those where R_{C} is hydrogen, halogen, C₁-C₁₀ alkyl, C₁-C₁₀ haloalkyl, C₃-C₇ cycloalkyl, or C₃-C₇ cycloalkyl(C₁-C₁₀)alkyl.

Even more preferred compounds of formula VIa and VIb are those where R_{C} is hydrogen, halogen, C₁-C₃ alkyl, C₁-C₃ haloalkyl, cycloopropyl, or cyclopropylmethyl.

Even more preferred compounds of formula VIa and VIb are those where R_{C} is independently hydrogen, halogen, methyl, ethyl, fluoromethyl, difluoromethyl, trifluoromethyl, cyclopropyl, or cyclopropylmethyl.

In another embodiment, the invention provides compounds according to formula (VIIa) and (VIIb), 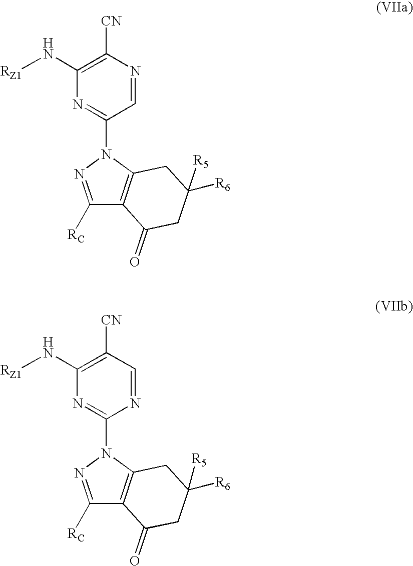
wherein R₅, R₆, R_{C}, and R_{Z1} are as defined for formula (I).

Preferred compounds of Formula VIIa and VIIb include those where R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, or R₂₃.

More preferred compounds of Formula VIIa and VIIb include those where R_{Z1} is a C₁-C₁₄ alkyl group where up to five of the carbon atoms in the alkyl group are optionally replaced independently by R₂₂, carbonyl, N, or O, with the proviso that two O atoms are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, or R₂₃.

Even more preferred compounds of formula VIIa and VIIb are those where R_{C} is hydrogen, halogen, C₁-C₁₀ alkyl, C₁-C₁₀ haloalkyl, C₃-C₇ cycloalkyl, or C₃-C₇ cycloalkyl(C₁-C₁₀)alkyl.

Even more preferred compounds of formula VIIa and VIIb are those where R_{C} is hydrogen, halogen, C₁-C₃ alkyl, C₁-C₃ haloalkyl, cycloopropyl, or cyclopropylmethyl.

Even more preferred compounds of formula VIIa and VIIb are those where R_{C} is independently hydrogen, halogen, methyl, ethyl, fluoromethyl, difluoromethyl, trifluoromethyl, cyclopropyl, or cyclopropylmethyl.

In another embodiment, the invention provides compounds according to formula (VIIIa) and (VIIIb), 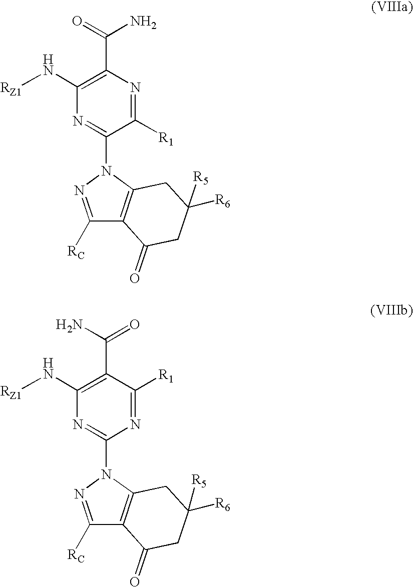
wherein R₁, R₅, R₆, R_{C}, and R_{Z1} are as defined for formula (I).

Preferred compounds of formula VIIIa and VIIIb are those where R₁ is hydrogen, halogen, amino, C₁-C₁₀ alkyl, or C₁-C₁₀ haloalkyl.

Preferred compounds of Formula VIIIa and VIIIb include those where R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, or R₂₃.

More preferred compounds of Formula VIIIa and VIIIb include those where R_{Z1} is a C₁-C₁₄ alkyl group where up to five of the carbon atoms in the alkyl group are optionally replaced independently by R₂₂, carbonyl, N, or O, with the proviso that two O atoms are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, or R₂₃.

Even more preferred compounds of formula VIIIa and VIIIb are those where R_{C} is hydrogen, halogen, C₁-C₁₀ alkyl, C₁-C₁₀ haloalkyl, C₃-C₇ cycloalkyl, or C₃-C₇ cycloalkyl(C₁-C₁₀)alkyl.

Even more preferred compounds of formula VIIIa and VIIIb are those where R_{C} is hydrogen, halogen, C₁-C₃ alkyl, C₁-C₃ haloalkyl, cycloopropyl, or cyclopropylmethyl.

Even more preferred compounds of formula VIIIa and VIIIb are those where R_{C} is independently hydrogen, halogen, methyl, ethyl, fluoromethyl, difluoromethyl, trifluoromethyl, cyclopropyl, or cyclopropylmethyl.

In another embodiment, the invention provides compounds according to formula (IXa) and (IXb), 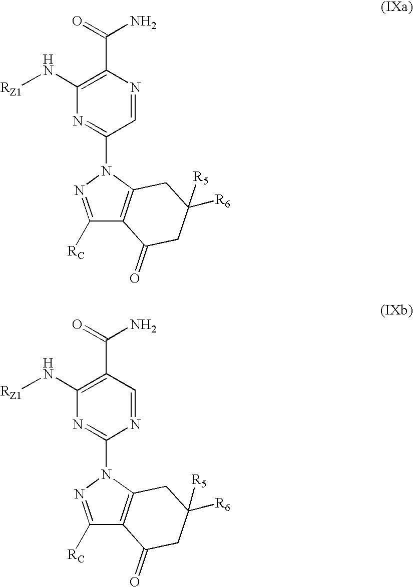
wherein R₅, R₆, R_{C}, and R_{Z1} are as defined for formula (I).

Preferred compounds of Formula IXa and IXb include those where R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, or R₂₃.

More preferred compounds of Formula IXa and IXb include those where R_{Z1} is a C₁-C₁₄ alkyl group where up to five of the carbon atoms in the alkyl group are optionally replaced independently by R₂₂, carbonyl, N, or O, with the proviso that two O atoms are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, or R₂₃.

Even more preferred compounds of formula IXa and IXb are those where R_{C} is hydrogen, halogen, C₁-C₁₀ alkyl, C₁-C₁₀ haloalkyl, C₃-C₇ cycloalkyl, or C₃-C₇ cycloalkyl(C₁-C₁₀)alkyl.

Even more preferred compounds of formula IXa and IXb are those where R_{C} is hydrogen, halogen, C₁-C₃ alkyl, C₁-C₃ haloalkyl, cycloopropyl, or cyclopropylmethyl.

Even more preferred compounds of formula IXa and IXb are those where R_{C} is independently hydrogen, halogen, methyl, ethyl, fluoromethyl, difluoromethyl, trifluoromethyl, cyclopropyl, or cyclopropylmethyl.

In another embodiment, the invention provides compounds according to formula (Xa) and (Xb), 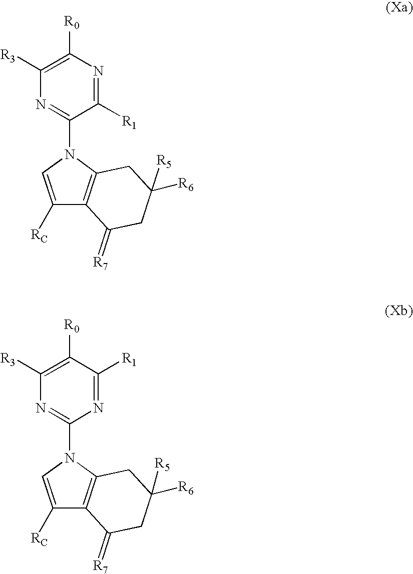
wherein R₀, R₁, R₃, R₅, R₆, R₇, and R_{C} are as defined for formula (I).

Preferred compounds of formula Xa and Xb include those where R₇ is O or N—OH. More preferred compounds of formula Xa and Xb are those wherein R₇ is O.

Other preferred compounds of formula Xa and Xb are those where R₁ is hydrogen, halogen, amino, C₁-C₁₀ alkyl, or C₁-C₁₀ haloalkyl.

Other preferred compounds of formula Xa and Xb are those where R₀ is cyano, hydroxyl, nitro, amino, mono- or di-(C₁-C₁₀)alkylamino, or a group of the formula 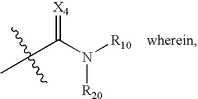
X₄ is O, NH, NOH, or S; and
R₁₀ and R₂₀ are independently H, OH, C₁-C₁₀ haloalkyl, or C₁-C₁₀alkyl.

More preferred compounds of formula Xa and Xb are those wherein R₀ is cyano or —CONH₂. More preferred compounds of formula Xa and Xb are those wherein R₀ is cyano. More preferred compounds of formula Xa and Xb are those wherein R₀ is —CONH₂.

Preferred compounds of Formula Xa and Xb include those where R₃ is hydrogen, halo, or -Z₁R_{Z1}, wherein Z₁ is —O—, —NH—, —S(O)ₚ—, or —S(O)₂NH—, wherein p is 0, 1 or 2; and R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S—(C₁-C₆)alkyl, —SO₂—(C₁-C₆)alkyl, —SO₂NH₂, —SO₂NH—(C₁-C₆)alkyl, —SO₂NH-aryl, —SO₂-aryl, —SO—(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, or R₂₃.

Even more preferred compounds of Formula Xa and Xb include those where R₃ is hydrogen, halo, or -Z₁R_{Z1}, wherein Z₁ is —O— or —NH—; and R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S—(C₁-C₆)alkyl, —SO₂—(C₁-C₆)alkyl, —SO₂NH₂, —SO₂NH—(C₁-C₆)alkyl, —SO₂NH-aryl, —SO₂-aryl, —SO—(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, or R₂₃.

Additional preferred compounds of Formula Xa and Xb include those where R₃ is hydrogen, halo, or —N(H)R_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S—(C₁-C₆)alkyl, —SO₂—(C₁-C₆)alkyl, —SO₂NH₂, —SO₂NH—(C₁-C₆)alkyl, —SO₂NH-aryl, —SO₂-aryl, —SO—(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, or R₂₃.

Most preferred compounds of Formula Xa and Xb include those where R₃ is hydrogen, halo, or —N(H)R_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, or R₂₃.

Additional preferred compounds of Formula Xa and Xb include those where R₃ is hydrogen, halo, or —OR_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S—(C₁-C₆)alkyl, —SO₂—(C₁-C₆)alkyl, —SO₂NH₂, —SO₂NH—(C₁-C₆)alkyl, —SO₂NH-aryl, —SO₂-aryl, —SO—(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, or R₂₃.

Most preferred compounds of Formula Xa and Xb include those where R₃ is hydrogen, halo, or —OR_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, or R₂₃.

Even more preferred compounds of formula Xa and Xb are those where R_{C} is hydrogen, halogen, C₁-C₁₀ alkyl, C₁-C₁₀ haloalkyl, C₃-C₇ cycloalkyl, or C₃-C₇ cycloalkyl(C₁-C₁₀)alkyl.

Even more preferred compounds of formula Xa and Xb are those where R_{C} is hydrogen, halogen, C₁-C₃ alkyl, C₁-C₃ haloalkyl, cycloopropyl, or cyclopropylmethyl.

Even more preferred compounds of formula Xa and Xb are those where R_{C} is independently hydrogen, halogen, methyl, ethyl, fluoromethyl, difluoromethyl, trifluoromethyl, cyclopropyl, or cyclopropylmethyl.

In another embodiment, the invention provides compounds according to formula (XIa) and (XIb), 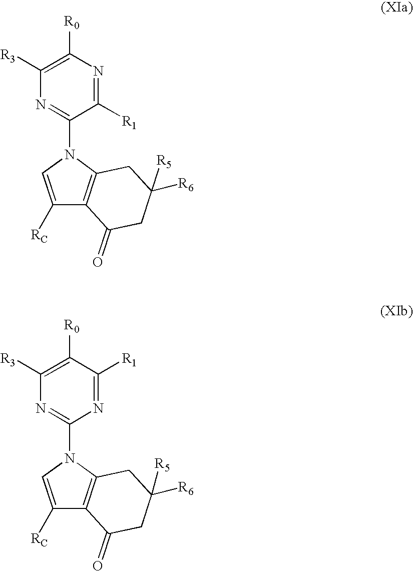
wherein R₀, R₁, R₃, R₅, R₆, and R_{C} are as defined for formula (I).

Preferred compounds of formula XIa and XIb are those where R₁ is hydrogen, halogen, amino, C₁-C₁₀ alkyl, or C₁-C₁₀ haloalkyl.

Other preferred compounds of formula XIa and XIb are those where R₀ is cyano, hydroxyl, nitro, amino, mono- or di-(C₁-C₁₀)alkylamino, or a group of the formula 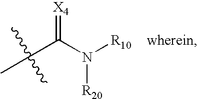
X₄ is O, NH, NOH, or S; and
R₁₀ and R₂₀ are independently H, OH, C₁-C₁₀ haloalkyl, or C₁-C₁₀alkyl.

More preferred compounds of formula XIa and XIb are those wherein R₀ is cyano or —CONH₂. More preferred compounds of formula XIa and XIb are those wherein R₀ is cyano. More preferred compounds of formula XIa and XIb are those wherein R₀ is —CONH₂.

Preferred compounds of Formula XIa and XIb include those where R₃ is hydrogen, halo, or -Z₁R_{Z1}, wherein Z₁ is —O—, —NH—, —S(O)ₚ—, or —S(O)₂NH—, wherein p is 0, 1 or 2; and R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S—(C₁-C₆)alkyl, —SO₂—(C₁-C₆)alkyl, —SO₂NH₂, —SO₂NH—(C₁-C₆)alkyl, —SO₂NH-aryl, —SO₂-aryl, —SO—(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, or R₂₃.

Even more preferred compounds of Formula XIa and XIb include those where R₃ is hydrogen, halo, or -Z₁R_{Z1}, wherein Z₁ is —O— or —NH—; and R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S—(C₁-C₆)alkyl, —SO₂—(C₁-C₆)alkyl, —SO₂NH₂, —SO₂NH—(C₁-C₆)alkyl, —SO₂NH-aryl, —SO₂-aryl, —SO—(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, or R₂₃.

Additional preferred compounds of Formula XIa and XIb include those where R₃ is hydrogen, halo, or —N(H)R_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S—(C₁-C₆)alkyl, —SO₂—(C₁-C₆)alkyl, —SO₂NH₂, —SO₂NH—(C₁-C₆)alkyl, —SO₂NH-aryl, —SO₂-aryl, —SO—(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, or R₂₃.

Most preferred compounds of Formula XIa and XIb include those where R₃ is hydrogen, halo, or —N(H)R_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, or R₂₃.

Additional preferred compounds of Formula XIa and XIb include those where R₃ is hydrogen, halo, or —OR_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S—(C₁-C₆)alkyl, —SO₂—(C₁-C₆)alkyl, —SO₂NH₂, —SO₂NH—(C₁-C₆)alkyl, —SO₂NH-aryl, —SO₂-aryl, —SO—(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, or R₂₃.

Most preferred compounds of Formula XIa and XIb include those where R₃ is hydrogen, halo, or —OR_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, or R₂₃.

Even more preferred compounds of formula XIa and XIb are those where R_{C} is hydrogen, halogen, C₁-C₁₀ alkyl, C₁-C₁₀ haloalkyl, C₃-C₇ cycloalkyl, or C₃-C₇ cycloalkyl(C₁-C₁₀)alkyl.

Even more preferred compounds of formula XIa and XIb are those where R_{C} is hydrogen, halogen, C₁-C₃ alkyl, C₁-C₃ haloalkyl, cycloopropyl, or cyclopropylmethyl.

Even more preferred compounds of formula XIa and XIb are those where R_{C} is independently hydrogen, halogen, methyl, ethyl, fluoromethyl, difluoromethyl, trifluoromethyl, cyclopropyl, or cyclopropylmethyl.

In another embodiment, the invention provides compounds according to formula (XIIa) and (XIIb), 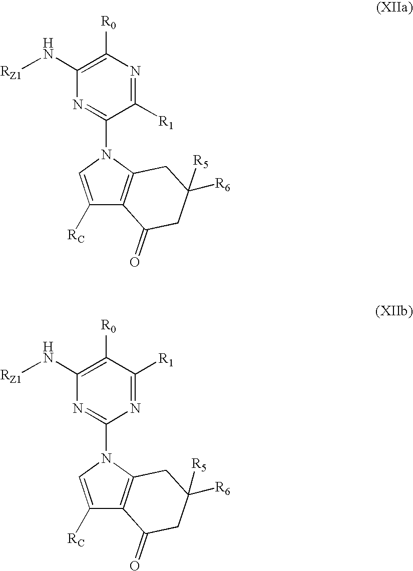
wherein R₀, R₁, R₅, R₆, R_{C}, and R_{Z1} are as defined for formula (I).

Preferred compounds of formula XIIa and XIIb are those where R₁ is hydrogen, halogen, amino, C₁-C₁₀ alkyl, or C₁-C₁₀ haloalkyl.

Other preferred compounds of formula XIIa and XIIb are those where R₀ is cyano, hydroxyl, nitro, amino, mono- or di-(C₁-C₁₀)alkylamino, or a group of the formula 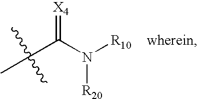
X₄ is O, NH, NOH, or S; and
R₁₀ and R₂₀ are independently H, OH, C₁-C₁₀ haloalkyl, or C₁-C₁₀alkyl.

More preferred compounds of formula XIIa and XIIb are those wherein R₀ is cyano or —CONH₂. More preferred compounds of formula XIIa and XIIb are those wherein R₀ is cyano. More preferred compounds of formula XIIa and XIIb are those wherein R₀ is —CONH₂.

Preferred compounds of Formula XIIa and XIIb include those where R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, or R₂₃.

More preferred compounds of Formula XIIa and XIIb include those where R_{Z1} is a C₁-C₁₄ alkyl group where up to five of the carbon atoms in the alkyl group are optionally replaced independently by R₂₂, carbonyl, N, or O, with the proviso that two O atoms are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, or R₂₃.

Even more preferred compounds of formula XIIa and XIIb are those where R_{C} is hydrogen, halogen, C₁-C₁₀ alkyl, C₁-C₁₀ haloalkyl, C₃-C₇ cycloalkyl, or C₃-C₇ cycloalkyl(C₁-C₁₀)alkyl.

Even more preferred compounds of formula XIIa and XIIb are those where R_{C} is hydrogen, halogen, C₁-C₃ alkyl, C₁-C₃ haloalkyl, cycloopropyl, or cyclopropylmethyl.

Even more preferred compounds of formula XIIa and XIIb are those where R_{C} is independently hydrogen, halogen, methyl, ethyl, fluoromethyl, difluoromethyl, trifluoromethyl, cyclopropyl, or cyclopropylmethyl.

In another embodiment, the invention provides compounds according to formula (XIIIa) and (XIIIb), 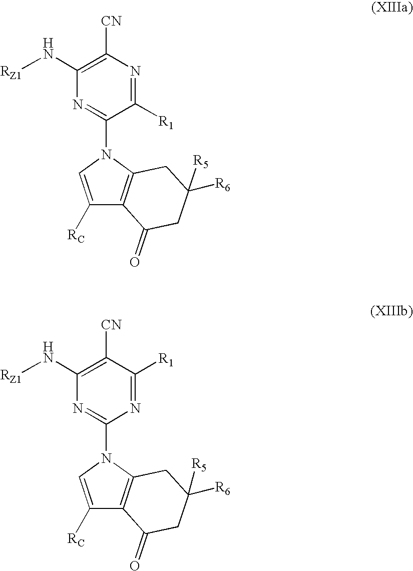
wherein R₁, R₅, R₆, R_{C}, and R_{Z1} are as defined for formula (I).

Preferred compounds of formula XIIIa and XIIIb are those where R₁ is hydrogen, halogen, amino, C₁-C₁₀ alkyl, or C₁-C₁₀ haloalkyl.

Preferred compounds of Formula XIIIa and XIIIb include those where R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, or R₂₃.

More preferred compounds of Formula XIIIa and XIIIb include those where R_{Z1} is a C₁-C₁₄ alkyl group where up to five of the carbon atoms in the alkyl group are optionally replaced independently by R₂₂, carbonyl, N, or O, with the proviso that two O atoms are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, or R₂₃.

Even more preferred compounds of formula XIIIa and XIIIb are those where R_{C} is hydrogen, halogen, C₁-C₁₀ alkyl, C₁-C₁₀ haloalkyl, C₃-C₇ cycloalkyl, or C₃-C₇ cycloalkyl(C₁-C₁₀)alkyl.

Even more preferred compounds of formula XIIIa and XIIIb are those where R_{C} is hydrogen, halogen, C₁-C₃ alkyl, C₁-C₃ haloalkyl, cycloopropyl, or cyclopropylmethyl.

Even more preferred compounds of formula XIIIa and XIIIb are those where R_{C} is independently hydrogen, halogen, methyl, ethyl, fluoromethyl, difluoromethyl, trifluoromethyl, cyclopropyl, or cyclopropylmethyl.

In another embodiment, the invention provides compounds according to formula (XIVa) and (XIVb), 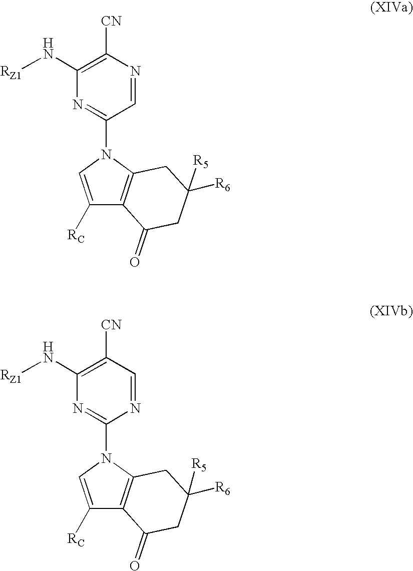
wherein R₅, R₆, R_{C}, and R_{Z1} are as defined for formula (I).

Preferred compounds of Formula XIVa and XIVb include those where R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, or R₂₃.

More preferred compounds of Formula XIVa and XIVb include those where R_{Z1} is a C₁-C₁₄ alkyl group where up to five of the carbon atoms in the alkyl group are optionally replaced independently by R₂₂, carbonyl, N, or O, with the proviso that two O atoms are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, or R₂₃.

Even more preferred compounds of formula XIVa and XIVb are those where R_{C} is hydrogen, halogen, C₁-C₁₀ alkyl, C₁-C₁₀ haloalkyl, C₃-C₇ cycloalkyl, or C₃-C₇ cycloalkyl(C₁-C₁₀)alkyl.

Even more preferred compounds of formula XIVa and XIVb are those where R_{C} is hydrogen, halogen, C₁-C₃ alkyl, C₁-C₃ haloalkyl, cycloopropyl, or cyclopropylmethyl.

Even more preferred compounds of formula XIVa and XIVb are those where R_{C} is independently hydrogen, halogen, methyl, ethyl, fluoromethyl, difluoromethyl, trifluoromethyl, cyclopropyl, or cyclopropylmethyl.

In another embodiment, the invention provides compounds according to formula (XVa) and (XVb), 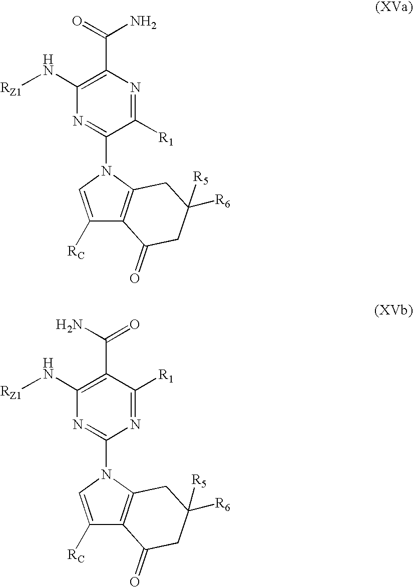
wherein R₁, R₅, R₆, R_{C}, and R_{Z1} are as defined for formula (I).

Preferred compounds of formula XVa and XVb are those where R₁ is hydrogen, halogen, amino, C₁-C₁₀ alkyl, or C₁-C₁₀ haloalkyl.

Preferred compounds of Formula XVa and XVb include those where R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, or R₂₃.

More preferred compounds of Formula XVa and XVb include those where R_{Z1} is a C₁-C₁₄ alkyl group where up to five of the carbon atoms in the alkyl group are optionally replaced independently by R₂₂, carbonyl, N, or O, with the proviso that two O atoms are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, or R₂₃.

Even more preferred compounds of formula XVa and XVb are those where R_{C} is hydrogen, halogen, C₁-C₁₀ alkyl, C₁-C₁₀ haloalkyl, C₃-C₇ cycloalkyl, or C₃-C₇ cycloalkyl(C₁-C₁₀)alkyl.

Even more preferred compounds of formula XVa and XVb are those where R_{C} is hydrogen, halogen, C₁-C₃ alkyl, C₁-C₃ haloalkyl, cycloopropyl, or cyclopropylmethyl.

Even more preferred compounds of formula XVa and XVb are those where R_{C} is independently hydrogen, halogen, methyl, ethyl, fluoromethyl, difluoromethyl, trifluoromethyl, cyclopropyl, or cyclopropylmethyl.

In another embodiment, the invention provides compounds according to formula (XVIa) and (XVIb), 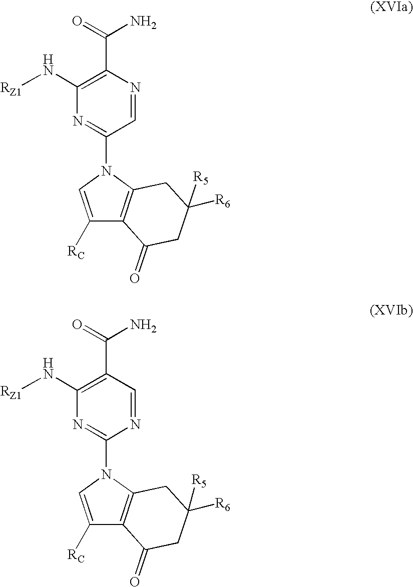
wherein R₅, R₆, R_{C}, and R_{Z1} are as defined for formula (I).

Preferred compounds of Formula XVIa and XVIb include those where R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, or R₂₃.

More preferred compounds of Formula XVIa and XVIb include those where R_{Z1} is a C₁-C₁₄ alkyl group where up to five of the carbon atoms in the alkyl group are optionally replaced independently by R₂₂, carbonyl, N, or O, with the proviso that two O atoms are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, or R₂₃.

Even more preferred compounds of formula XVIa and XVIb are those where R_{C} is hydrogen, halogen, C₁-C₁₀ alkyl, C₁-C₁₀ haloalkyl, C₃-C₇ cycloalkyl, or C₃-C₇ cycloalkyl(C₁-C₁₀)alkyl.

Even more preferred compounds of formula XVIa and XVIb are those where R_{C} is hydrogen, halogen, C₁-C₃ alkyl, C₁-C₃ haloalkyl, cycloopropyl, or cyclopropylmethyl.

Even more preferred compounds of formula XVIa and XVIb are those where R_{C} is independently hydrogen, halogen, methyl, ethyl, fluoromethyl, difluoromethyl, trifluoromethyl, cyclopropyl, or cyclopropylmethyl.

In a second aspect, the invention encompasses a method of treating cancer comprising administering to a patient in need thereof, a pharmaceutically acceptable amount of a compound or salt of any of Formulas I, IIa-XVIa, or IIb-XVIb or a pharmaceutical composition comprising a compound or salt of pharmaceutically acceptable amount of a compound or salt of any of Formulas I, IIa-XVIa, or IIb-XVIb.

In a preferred embodiment of the second aspect, the invention encompasses a method of treating cancer comprising administering to a patient in need thereof, a pharmaceutically acceptable amount of a compound or salt of Formula I or a pharmaceutical composition comprising a compound or salt of Formula I.

In a third aspect, the invention encompasses the use of a therapeutically effective amount of a compound or salt of any of Formulas I, IIa-XVIa, or IIb-XVIb for the preparation of a medicament for the treatment of cancer, inflammation, or arthritis in a patient in need of such treatment.

In a preferred embodiment of the third aspect, the invention encompasses the use of a therapeutically effective amount of a compound or salt of Formula I for the preparation of a medicament for the treatment of cancer, inflammation, or arthritis in a patient in need of such treatment.

In a fourth aspect, the invention encompasses a package comprising a compound or salt of any of Formulas I, IIa-XVIa, or IIb-XVIb in a container with instructions on how to use the compound.

In a preferred embodiment of the fourth aspect, the invention encompasses a package comprising a compound or salt of Formula I in a container with instructions on how to use the compound.

In a fifth aspect, the invention encompasses the use of a therapeutically effective amount of a compound or salt according to any of Formulas I, IIa-XVIa, or IIb-XVIb for the preparation of a medicament for the treatment of a disease or condition related to cell proliferation in a patient in need of such treatment.

In a preferred embodiment of the fifth aspect, the invention encompasses the use of a therapeutically effective amount of a compound or salt according to Formula I for the preparation of a medicament for the treatment of a disease or condition related to cell proliferation in a patient in need of such treatment.

In a sixth aspect, the invention encompasses the use of a therapeutically effective amount of a compound or salt according according to any of Formulas I, IIa-XVIa, or IIb-XVIb for the preparation of a medicament for the treatment of a disease or condition related to cell proliferation in a patient in need of such treatment, wherein the disease or condition is cancer, inflammation, or arthritis.

In a preferred embodiment of the sixth aspect, the invention encompasses the use of a therapeutically effective amount of a compound or salt according to Formula I for the preparation of a medicament for the treatment of a disease or condition related to cell proliferation in a patient in need of such treatment, wherein the disease or condition is cancer, inflammation, or arthritis.

In a seventh aspect, the invention encompasses the use of therapeutically effective amount of a compound or salt of any of Formulas I, IIa-XVIa, or IIb-XVIb for the preparation of a medicament for the treatment of a disease or disorder related to the activity of heat shock protein 90, in a subject in need of such.

In a preferred embodiment of the seventh aspect, the invention encompasses the use of therapeutically effective amount of a compound or salt of Formula I for the preparation of a medicament for the treatment of a disease or disorder related to the activity of heat shock protein 90, in a subject in need of such.

In a eighth aspect, the invention encompasses the use of therapeutically effective amount of a compound or salt of any of Formulas I, IIa-XVIa, or IIb-XVIb, alone or in combination with another therapeutic agent, for the preparation of a medicament for the treatment of a disease or disorder related to the activity of heat shock protein 90 and/or its client proteins, in a subject in need of such, wherein the HSP-90 mediated disorder is selected from the group of inflammatory diseases, infections, autoimmune disorders, stroke, ischemia, cardiac disorders, neurological disorders, fibrogenetic disorders, proliferative disorders, tumors, leukemias, neoplasms, cancers, carcinomas, metabolic diseases and malignant disease.

In a preferred embodiment of the eighth aspect, the invention encompasses the use of therapeutically effective amount of a compound or salt of Formula I, alone or in combination with another therapeutic agent, for the preparation of a medicament for the treatment of a disease or disorder related to the activity of heat shock protein 90 and/or its client proteins, in a subject in need of such, wherein the HSP-90 mediated disorder is selected from the group of inflammatory diseases, infections, autoimmune disorders, stroke, ischemia, cardiac disorders, neurological disorders, fibrogenetic disorders, proliferative disorders, tumors, leukemias, neoplasms, cancers, carcinomas, metabolic diseases and malignant disease.

In a preferred aspect embodiment of the eighth aspect, the invention encompasses methods for the treatment of cancer in a subject in need of such treatment comprising administration of therapeutically effective amount of a compound or salt of Formula I, in combination with at least one other therapeutic agent.

In a more preferred aspect embodiment of the eighth aspect, the invention encompasses methods for treating cancer in a subject in need of such treatment, the methods comprising administration of therapeutically effective amount of a compound or salt of Formula I, in combination with at least one other anti-cancer agent.

In another preferred aspect embodiment of the eighth aspect, the invention encompasses methods for treating cancer, the methods comprising administration, to a subject in need of such treatment, of a therapeutically effective amount of a compound or salt of Formula I, in combination with radiation therapy.

In a ninth aspect, the invention encompasses the use of therapeutically effective amount of a compound or salt of any of Formulas I, IIa-XVIa, or IIb-XVIb for the preparation of a medicament for the treatment of a fibrogenetic disorder related to the activity of heat shock protein 90, in a subject in need of such, wherein the fibrogenetic disorder is selected from the group of scleroderma, polymyositis, systemic lupus, rheumatoid arthritis, liver cirrhosis, keloid formation, interstitial nephritis and pulmonary fibrosis.

In a tenth aspect, the invention encompasses the use of a therapeutically effective amount of a compound or salt of any of Formulas I, IIa-XVIa, or IIb-XVIb for the preparation of a medicament for protecting a subject from infection caused by an organism selected from Plasmodium species.

In a preferred embodiment of the tenth aspect, the invention encompasses the use of a therapeutically effective amount of a compound or salt of Formula I for the preparation of a medicament for protecting a subject from infection caused by Plasmodium falciparum.

In an eleventh aspect, the invention encompasses the use of a therapeutically effective amount of a compound or salt of any of Formulas I, IIa-XVIa, or IIb-XVIb for the preparation of a medicament for reducing the level of infection caused by an organism selected from Plasmodium species in a subject in need of such treatment.

In a preferred embodiment of the eleventh aspect, the invention encompasses the use of a therapeutically effective amount of a compound or salt of Formula I for the preparation of a medicament for reducing the level of infection caused by an organism selected from Plasmodium species in a subject in need of such treatment.

In a preferred aspect of the eleventh aspect, the invention encompasses the use of a therapeutically effective amount of a compound or salt of Formula I for the preparation of a medicament for reducing the level of infection caused by Plasmodium falciparum in a subject in need of such treatment

In a twelfth aspect, the invention encompasses the use of a therapeutically effective amount of a compound or salt of any of Formulas I, IIa-XVIa, or IIb-XVIb for the preparation of a medicament for treating a patient infected with a metazoan parasite.

In a preferred embodiment of the twelfth aspect, the invention encompasses the use of a therapeutically effective amount of a compound or salt of Formula I for the preparation of a medicament for treating a patient infected with a metazoan parasite.

In a more preferred embodiment of the twelfth aspect, the invention encompasses the use of a therapeutically effective amount of a compound or salt of Formula I for the preparation of a medicament for treating a patient infected by a metazoan parasite which is Plasmodium falciparum.

In a thirteenth aspect, the invention encompasses the use of a therapeutically effective amount of a compound or salt of any of Formulas I, IIa-XVIa, or IIb-XVIb in combination with one or more known anti-fungal drugs for the preparation of a medicament for treating a patient infected with a fungal infection.

In a preferred embodiment of the thirteenth aspect, the invention encompasses the use of a therapeutically effective amount of a compound or salt of Formula I in combination with one or more known anti-fungal drugs for the preparation of a medicament for treating a patient infected with a fungal infection.

In the methods for treating viral infections, particular viral infections include those resulting from HIV-1 and Hepatitis C virus.

### DEFINITIONS

In Formula I, R₃ is, as noted above, independently (a) hydrogen, (b) halo, or (c) an alkyl group having from 1-15 carbon atoms. All, but no more than about six, of the carbon atoms in the alkyl group may be replaced independently by the various groups listed above in connection with Formula I. Replacement of any carbon atom is permitted, i.e., both internal and terminal carbon atoms. Further, the alkyl groups of from 1-15 carbon atoms may be straight or branched.

Thus, when the alkyl group is methyl, i.e., a one carbon atom alkyl group, replacement of that carbon atom with, for example, nitrogen or sulfur, the resulting group will not be an alkyl group but instead will be an amino or thio group, respectively. Similarly, when the carbon atom being replaced terminates the alkyl group, the terminal group will become another moiety such as pyrimidinyl, amino, phenyl, or hydroxy.

Replacement of a carbon atom with a group such as, for example, oxygen, nitrogen, or sulfur will require appropriate adjustment of the number of hydrogens or other atoms required to satisfy the replacing atom's valency. Thus, when the replacement is N or O, the number of groups attached to the atom being replaced will be reduced by one or two to satisfy the valency of the nitrogen or oxygen respectively. Similar considerations will be readily apparent to those skilled in the art with respect to replacement by ethenyl and ethynyl.

Thus, replacement as permitted herein results in the term “C₁-C₁₅ alkyl” as defined in connection with Formula I encompassing groups such as, but not limited to: amino, hydroxy, phenyl, benzyl, propylaminoethoxy, butoxyethylamino, pyrid-2-ylpropyl, diethylaminomethyl, pentylsulfonyl, methylsulfonamidoethyl, 3-[4-(butylpyrimidin-2-yl)ethyl]phenyl, butoxy, dimethylamino, 4-(2-(benzylamino)ethyl)pyridyl, but-2-enylamino, 4-(1-(methylamino)pent-3-en-2-ylthio)phenyl, 2-(N-methyl-hexanamido)ethoxy)methyl, and 4-(((3-methoxy-4-(4-methyl-1H-imidazol-2-yl)but-1-enyl)(methyl)amino)-methyl)phenyl.

Further, replacement as permitted herein may result in an R₃ group that exceeds 15 atoms. For example, replacing 6 carbon atoms of a 11-carbon atom straight chain alkyl group with amino, tetrahydropyran, amino, chlorophenyl, imidazolyl, and hydroxy could result in an R₃ group of the formula:

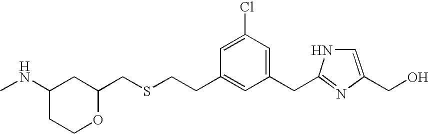

The term “alkoxy” represents an alkyl group of indicated number of carbon atoms attached to the parent molecular moiety through an oxygen bridge. Examples of alkoxy groups include, for example, methoxy, ethoxy, propoxy and isopropoxy.

As used herein, the term “alkyl” includes those alkyl groups of a designated number of carbon atoms. Alkyl groups may be straight, or branched. Examples of “alkyl” include methyl, ethyl, propyl, isopropyl, butyl, iso-, sec- and tert-butyl, pentyl, hexyl, heptyl, 3-ethylbutyl, and the like.

The term “alkenyl” as used herein, means a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkenoxy” refers to an alkenyl group attached to the parent group through an oxygen atom.

The term “alkynyl” as used herein, means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “aryl” refers to an aromatic hydrocarbon ring system containing at least one aromatic ring. The aromatic ring may optionally be fused or otherwise attached to other aromatic hydrocarbon rings or non-aromatic hydrocarbon rings. Examples of aryl groups include, for example, phenyl, naphthyl, 1,2,3,4-tetrahydronaphthalene and biphenyl. Preferred examples of aryl groups include phenyl, naphthyl, and anthracenyl. More preferred aryl groups are phenyl and naphthyl. Most preferred is phenyl. The aryl groups of the invention may be substituted with various groups as provided herein. Thus, any carbon atom present within an aryl ring system and available for substitution may be further bonded to a variety of ring substituents, such as, for example, halogen, hydroxy, nitro, cyano, amino, C₁-C₈alkyl, C₁-C₈alkoxy, mono- and di(C₁-C₈alkyl)amino, C₃-C₁₀cycloalkyl, (C₃-C₁₀cycloalkyl)alkyl, (C₃-C₁₀cycloalkyl)alkoxy, C₂-C₉heterocycloalkyl, C₁-C₈alkenyl, C₁-C₈alkynyl, halo(C₁-C₈)alkyl, halo(C₁-C₈)alkoxy, oxo, amino(C₁-C₈)alkyl, mono- and di(C₁-C₈alkyl)amino(C₁-C₈)alkyl, C₁-C₈acyl, C₁-C₈acyloxy, C₁-C₈sulfonyl, C₁-C₈thio, C₁-C₈sulfonamido, C₁-C₈-aminosulfonyl.

The term “carboxy” as used herein, means a —CO₂H group.

The term “cycloalkyl” refers to a C₃-C₈ cyclic hydrocarbon. Examples of cycloalkyl include cyclopropyl, cyclobutyl, cyclopentyl, cyclohexyl, cycloheptyl and cyclooctyl. More preferred are C₃-C₆ cycloalkyl groups. The cycloalkyl groups of the invention may be substituted with various groups as provided herein. Thus, any carbon atom present within a cycloalkyl ring system and available for substitution may be further bonded to a variety of ring substituents, such as, for example, halogen, hydroxy, nitro, cyano, amino, C₁-C₈alkyl, C₁-C₈alkoxy, mono- and di(C₁-C₈alkyl)amino, C₃-C₁₀cycloalkyl, (C₃-C₁₀cycloalkyl)alkyl, (C₃-C₁₀cycloalkyl)alkoxy, C₂-C₉heterocycloalkyl, C₁-C₈alkenyl, C₁-C₈alkynyl, halo(C₁-C₈)alkyl, halo(C₁-C₈)alkoxy, oxo, amino(C₁-C₈)alkyl and mono- and di(C₁-C₈alkyl)amino(C₁-C₈)alkyl.

The terms “halogen” or “halo” indicate fluorine, chlorine, bromine, and iodine.

The term “haloalkoxy” refers to an alkoxy group substituted with one or more halogen atoms, where each halogen is independently F, Cl, Br or I. Preferred halogens are F and Cl. Preferred haloalkoxy groups contain 1-6 carbons, more preferably 1-4 carbons, and still more preferably 1-2 carbons. “Haloalkoxy” includes perhaloalkoxy groups, such as OCF₃ or OCF₂CF₃. A preferred haloalkoxy group is trifluoromethoxy.

The term “haloalkyl” refers to an alkyl group substituted with one or more halogen atoms, where each halogen is independently F, Cl, Br or I. Preferred halogens are F and Cl. Preferred haloalkyl groups contain 1-6 carbons, more preferably 1-4 carbons, and still more preferably 1-2 carbons. “Haloalkyl” includes perhaloalkyl groups, such as CF₃ or CF₂CF₃. A preferred haloalkyl group is trifluoromethyl.

The term “heterocycloalkyl” refers to a ring or ring system containing at least one heteroatom selected from nitrogen, oxygen, and sulfur, wherein said heteroatom is in a non-aromatic ring. The heterocycloalkyl ring is optionally fused to or otherwise attached to other heterocycloalkyl rings and/or non-aromatic hydrocarbon rings and/or phenyl rings. To clarify the nomemclature herein, by C₃-C₁₀ heterocycloalkyl is meant such a ring containing from 3-10 ring members and those members are selected from carbon and hetero atoms such as oxygen, nitrogen, and sulfur. Preferred heterocycloalkyl groups have from 3 to 7 members. More preferred heterocycloalkyl groups have 5 or 6 members. Examples of heterocycloalkyl groups include, for example, 1,2,3,4-tetrahydroisoquinolinyl, piperazinyl, morpholinyl, piperidinyl, tetrahydrofuranyl, pyrrolidinyl, pyridinonyl, and pyrazolidinyl. Preferred heterocycloalkyl groups include piperidinyl, piperazinyl, morpholinyl, pyrrolidinyl, pyridinonyl, dihydropyrrolidinyl, and pyrrolidinonyl. The heterocycloalkyl groups of the invention may be substituted with various groups as provided herein. Thus, any atom present within a heterocycloalkyl ring and available for substitution may be further bonded to a variety of ring substituents, such as, for example, halogen, hydroxy, nitro, cyano, amino, C₁-C₈alkyl, C₁-C₈alkoxy, mono- and di(C₁-C₈alkyl)amino, C₃-C₁₀cycloalkyl, (C₃-C₁₀cycloalkyl)alkyl, (C₃-C₁₀cycloalkyl)alkoxy, C₂-C₉heterocycloalkyl, C₁-C₈alkenyl, C₁-C₈alkynyl, halo(C₁-C₈)alkyl, halo(C₁-C₈)alkoxy, oxo, amino(C₁-C₈)alkyl and mono- and di(C₁-C₈alkyl)amino(C₁-C₈)alkyl.

The term “heteroaryl” refers to an aromatic ring system containing at least one heteroatom selected from nitrogen, oxygen, and sulfur. The heteroaryl ring may be fused or otherwise attached to one or more heteroaryl rings, aromatic or non-aromatic hydrocarbon rings or heterocycloalkyl rings. Examples of heteroaryl groups include, for example, pyridine, furan, thienyl, 5,6,7,8-tetrahydroisoquinoline and pyrimidines. The heteroaryl groups of the invention may be substituted with various groups as provided herein. Thus, any carbon atom present within an heteroaryl ring system and available for substitution may be further bonded to a variety of ring substituents, such as, for example, halogen, hydroxy, nitro, cyano, amino, C₁-C₈alkyl, C₁-C₈alkoxy, mono- and di(C₁-C₈alkyl)amino, C₃-C₁₀cycloalkyl, (C₃-C₁₀cycloalkyl)alkyl, (C₃-C₁₀cycloalkyl)alkoxy, C₂-C₉heterocycloalkyl, C₁-C₈alkenyl, C₁-C₈alkynyl, halo(C₁-C₈)alkyl, halo(C₁-C₈)alkoxy, oxo, amino(C₁-C₈)alkyl and mono- and di(C₁-C₈alkyl)amino(C₁-C₈)alkyl.

Preferred examples of heteroaryl groups include thienyl, benzothienyl, pyridyl, quinolyl, pyrazolyl, pyrimidyl, imidazolyl, benzimidazolyl, furanyl, benzofuranyl, dibenzofuranyl, thiazolyl, benzothiazolyl, isoxazolyl, oxadiazolyl, isothiazolyl, benzisothiazolyl, triazolyl, pyrrolyl, indolyl, pyrazolyl, and benzopyrazolyl.

The compounds of this invention may contain one or more asymmetric carbon atoms, so that the compounds can exist in different stereoisomeric forms. These compounds can be, for example, racemates, chiral non-racemic or diastereomers. In these situations, the single enantiomers, i.e., optically active forms, can be obtained by asymmetric synthesis or by resolution of the racemates. Resolution of the racemates can be accomplished, for example, by conventional methods such as crystallization in the presence of a resolving agent; chromatography, using, for example a chiral HPLC column; or derivatizing the racemic mixture with a resolving reagent to generate diastereomers, separating the diastereomers via chromatography, and removing the resolving agent to generate the original compound in enantiomerically enriched form. Any of the above procedures can be repeated to increase the enantiomeric purity of a compound.

When the compounds described herein contain olefinic double bonds or other centers of geometric asymmetry, and unless otherwise specified, it is intended that the compounds include the cis, trans, Z- and E-configurations. Likewise, all tautomeric forms are also intended to be included.

Pharmaceutical Compositions

The compounds of general Formula I may be administered orally, topically, parenterally, by inhalation or spray or rectally in dosage unit formulations containing conventional non-toxic pharmaceutically acceptable carriers, adjuvants and vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a compound of general Formula I and a pharmaceutically acceptable carrier. One or more compounds of general Formula I may be present in association with one or more non-toxic pharmaceutically acceptable carriers and/or diluents and/or adjuvants, and if desired other active ingredients. The pharmaceutical compositions containing compounds of general Formula I may be in a form suitable for oral use, for example, as tablets, troches, lozenges, aqueous or oily suspensions, dispersible powders or granules, emulsion, hard or soft capsules, or syrups or elixirs.

Compositions intended for oral use may be prepared according to any method known in the art for the manufacture of pharmaceutical compositions and such compositions may contain one or more agents selected from the group consisting of sweetening agents, flavoring agents, coloring agents and preservative agents in order to provide pharmaceutically elegant and palatable preparations. Tablets contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques. In some cases such coatings may be prepared by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatin capsules, wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Formulations for oral use may also be presented as lozenges.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, may also be present.

Pharmaceutical compositions of the invention may also be in the form of oil-in-water emulsions. The oily phase may be a vegetable oil or a mineral oil or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol, glucose or sucrose. Such formulations may also contain a demulcent, a preservative and flavoring and coloring agents. The pharmaceutical compositions may be in the form of a sterile injectable aqueous or oleaginous suspension. This suspension may be formulated according to the known art using those suitable dispersing or wetting agents and suspending agents that have been mentioned above. The sterile injectable preparation may also be a sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of general Formula I may also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Compounds of general Formula I may be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

For disorders of the eye or other external tissues, e.g., mouth and skin, the formulations are preferably applied as a topical gel, spray, ointment or cream, or as a suppository, containing the active ingredients in a total amount of, for example, 0.075 to 30% w/w, preferably 0.2 to 20% w/w and most preferably 0.4 to 15% w/w. When formulated in an ointment, the active ingredients may be employed with either paraffinic or a water-miscible ointment base.

Alternatively, the active ingredients may be formulated in a cream with an oil-in-water cream base. If desired, the aqueous phase of the cream base may include, for example at least 30% w/w of a polyhydric alcohol such as propylene glycol, butane-1,3-diol, mannitol, sorbitol, glycerol, polyethylene glycol and mixtures thereof. The topical formulation may desirably include a compound which enhances absorption or penetration of the active ingredient through the skin or other affected areas. Examples of such dermal penetration enhancers include dimethylsulfoxide and related analogs. The compounds of this invention can also be administered by a transdermal device. Preferably topical administration will be accomplished using a patch either of the reservoir and porous membrane type or of a solid matrix variety. In either case, the active agent is delivered continuously from the reservoir or microcapsules through a membrane into the active agent permeable adhesive, which is in contact with the skin or mucosa of the recipient. If the active agent is absorbed through the skin, a controlled and predetermined flow of the active agent is administered to the recipient. In the case of microcapsules, the encapsulating agent may also function as the membrane. The transdermal patch may include the compound in a suitable solvent system with an adhesive system, such as an acrylic emulsion, and a polyester patch. The oily phase of the emulsions of this invention may be constituted from known ingredients in a known manner. While the phase may comprise merely an emulsifier, it may comprise a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make-up the so-called emulsifying wax, and the wax together with the oil and fat make up the so-called emulsifying ointment base which forms the oily dispersed phase of the cream formulations. Emulsifiers and emulsion stabilizers suitable for use in the formulation of the invention include Tween 60, Span 80, cetostearyl alcohol, myristyl alcohol, glyceryl monostearate, and sodium lauryl sulfate, among others. The choice of suitable oils or fats for the formulation is based on achieving the desired cosmetic properties, since the solubility of the active compound in most oils likely to be used in pharmaceutical emulsion formulations is very low. Thus, the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters may be used. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredients are dissolved or suspended in suitable carrier, especially an aqueous solvent for the active ingredients. The antiinflammatory active ingredients are preferably present in such formulations in a concentration of 0.5 to 20%, advantageously 0.5 to 10% and particularly about 1.5% w/w. For therapeutic purposes, the active compounds of this combination invention are ordinarily combined with one or more adjuvants appropriate to the indicated route of administration. If administered per os, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose. Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The compounds may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Dosage levels of the order of from about 0.1 mg to about 140 mg per kilogram of body weight per day are useful in the treatment of the above-indicated conditions (about 0.5 mg to about 7 g per patient per day). The amount of active ingredient that may be combined with the carrier materials to produce a single dosage form will vary depending upon the host treated and the particular mode of administration. Dosage unit forms will generally contain between from about 1 mg to about 500 mg of an active ingredient. The daily dose can be administered in one to four doses per day. In the case of skin conditions, it may be preferable to apply a topical preparation of compounds of this invention to the affected area two to four times a day.

It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition may also be added to the animal feed or drinking water. It may be convenient to formulate the animal feed and drinking water compositions so that the animal takes in a therapeutically appropriate quantity of the composition along with its diet. It may also be convenient to present the composition as a premix for addition to the feed or drinking water. Preferred non-human animals include domesticated animals.

The compounds of the invention may be administered alone or in combination with at least one additional therapeutic agent or therapy, e.g., radiation therapy, to a patient in need of such treatment. The additional therapeutic agent or therapy may be administered at the same time, separately, or sequentially with respect to the administration of a compound of formula I. Such additional therapeutic agents included, but are not limited to, anti-cancer agents, anti-inflammatory agents, and the like.

The compounds of the invention may be prepared by use of known chemical reactions and procedures. Representative methods for synthesizing compounds of the invention are presented below. It is understood that the nature of the substituents required for the desired target compound often determines the preferred method of synthesis. All variable groups of these methods are as described in the generic description if they are not specifically defined below.

Methods of Preparation

General Procedure

Representative synthetic procedures for the preparation of compounds of the invention are outlined below in following schemes. Unless otherwise indicated, all variables carry the definitions given in connection with Formula I.

Those having skill in the art will recognize that the starting materials and reaction conditions may be varied, the sequence of the reactions altered, and additional steps employed to produce compounds encompassed by the invention, as demonstrated by the following examples. In some cases, protection of certain reactive functionalities may be necessary to achieve some of the above transformations. In general, the need for such protecting groups as well as the conditions necessary to attach and remove such groups will be apparent to those skilled in the art of organic synthesis.

The disclosures of all articles and references mentioned in this application, including patents, are incorporated herein by reference in their entirety.

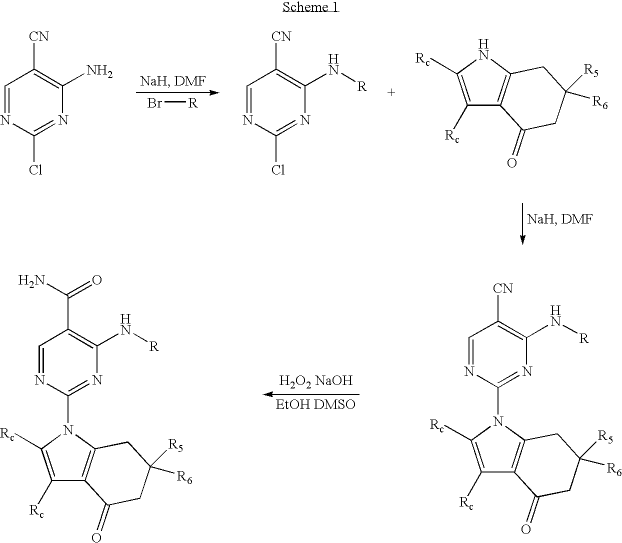

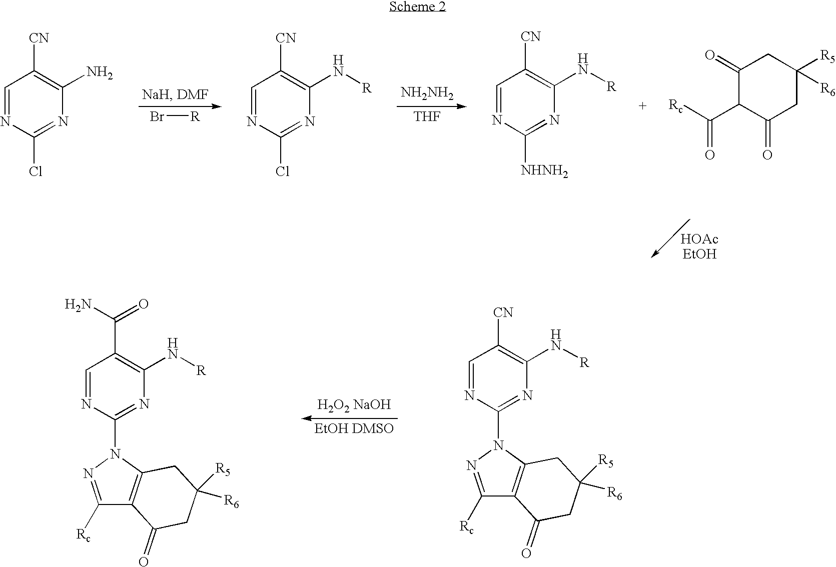

### EXAMPLES

The preparation of the compounds of the invention is illustrated further by the following examples, which are not to be construed as limiting the invention in scope or spirit to the specific procedures and compounds described in them. In all cases, unless otherwise specified, the column chromatography is performed using a silica gel solid phase.

### Example 1

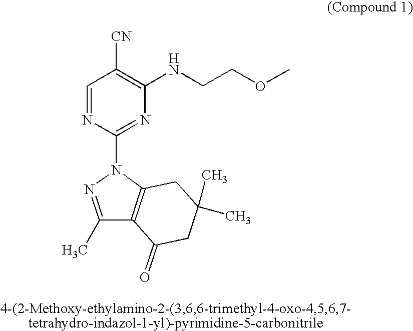 A solution of 4-amino-2-chloropyromidine-5-carbonitrile (0.101 g, 0.65 mmol), bromoethyl methyl ether (0.09 mL, 0.98 mmol), and NaH (60% suspension in mineral oil, 0.04 g, 0.98 mmol) in DMF (1 mL) is stirred at 80° C. in an oil bath for 3 h. Water is added (2 mL), and the aqueous phase is extracted with EtOAc (3×). The combined organic layers are washed with brine, dried over MgSO₄, and evaporated to dryness. Purification of the crude material using a Biotage column (0-50% EtOAc/hexanes for 5 CV, and 50% EtOAc/hexanes for 10 CV) affords 0.0721 g of 2-Chloro-4-(2-methoxy-ethylamino)-pyrimidine-5-carbonitrile (52%). LC/MS Calculated for C₈H₉ClN₄O: m/z=212.64. Found: m/z=213 [M+H]⁺.

To a solution of 2-Chloro-4-(2-methoxy-ethylamino)-pyrimidine-5-carbonitrile (0.14 g, 0.66 mmol) in THF (2 mL) is slowly added anhydrous hydrazine (0.26 mL) and the reaction mixture is stirred at RT for 2.5 d. A white solid formed upon addition of hydrazine. The white solid is dissolved in THF (20 mL). The organic layer is washed with NaHCO₃ (saturated) intil pH=8. The aqueous layer is extracted with EtOAc (2×). The combined organic layers are evaporated to afford 0.045 g (30%) of 2-Hydrazino-4-(2-methoxy-ethylamino)-pyrimidine-5-carbonitrile. LC/MS Calculated for C₈H₁₂N₆O: m/z=208. Found: m/z=209 [M+H]⁺.

A solution of 2-Hydrazino-4-(2-methoxy-ethylamino)-pyrimidine-5-carbonitrile (0.0415 g, 0.2 mmol) and 2-acetyl-5,5-dimethyl-1,3-cyclohexanedione (0.04 g, 0.22 mmol) in EtOH/Acetic Acid (3:1) (2 mL) is microwaved at 150° C. for 1000 s. The solvent is removed under reduced pressure and the residue is purified by column chromatography (20-80% EtOAc/hexanes for 6 CV) to yield 0.0296 g (42%) of 4-(2-Methoxy-ethylamino)-2-(3,6,6-trimethyl-4-oxo-4,5,6,7-tetrahydro-indazol-1-yl)-pyrimidine-5-carbonitrile. LC/MS Calculated for C₁₈H₂₂N₆O₂: m/z=354.4. Found: m/z=355.1 [M+H]⁺.

### Example 2

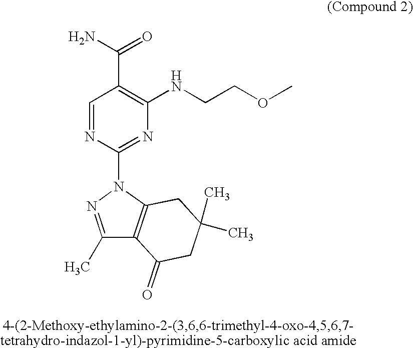 To a solution of 4-(2-Methoxy-ethylamino)-2-(3,6,6-trimethyl-4-oxo-4,5,6,7-tetrahydro-indazol-1-yl)-pyrimidine-5-carbonitrile (0.0296 g, 0.084 mmol) in 20% DMSO/EtOH (1 mL) are added 1 drop of NaOH (1 M), and 4 drops of H₂O₂. The reaction mixture is stirred at RT for 2 h. After addition of brine (10 mL) the aqueous phase is extracted with EtOAc (3×). The combined organic layers are dried over MgSO₄, and evaporated to afford 0.031 g (75%) of 4-(2-Methoxy-ethylamino)-2-(3,6,6-trimethyl-4-oxo-4,5,6,7-tetrahydro-indazol-1-yl) -pyrimidine-5-carboxylic acid amide. LC/MS Calculated for C₁₈H₂₄N₆O₃: m/z=372.4. Found: m/z=373.1 [M+H]⁺.

### Example 3

The following compounds are prepared essentially according to the procedures set forth in the above schemes and detailed in the preceding examples. Thus, the procedures for preparing the following compounds use the same or analogous synthetic techniques with substitution of alternative starting materials as necessary. Suitable variations and alternatives for preparing the following compounds will be readily apparent to those skilled in the art of organic synthesis in view of the procedures and examples herein.

| | | |
|---|---|---|
| 3 | 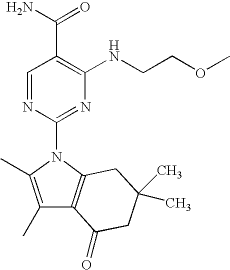 | 4-(2-methoxyethylamino)-2-(2,3,6,6- tetramethyl-4-oxo-4,5,6,7-tetrahydro- 1H-indol-1-yl)pyrimidine-5- carboxamide |
| 4 | 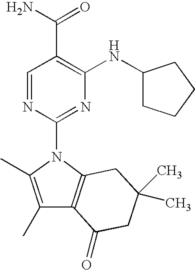 | 4-(cyclopentylamino)-2-(2,3,6,6- tetramethyl-4-oxo-4,5,6,7-tetrahydro- 1H-indol-1-yl)pyrimidine-5- carboxamide |
| 5 | 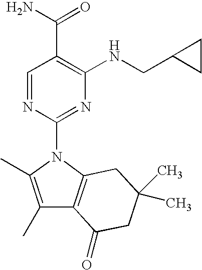 | 4-(cyclopropylmethylamino)-2- (2,3,6,6-tetramethyl-4-oxo-4,5,6,7- tetrahydro-1H-indol-1-yl)pyrimidine- 5-carboxamide |
| 6 | 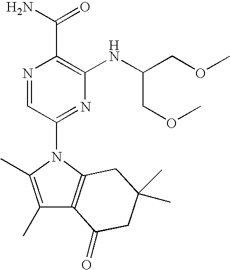 | 3-(1,3-dimethoxypropan-2-ylamino)-5- (2,3,6,6-tetramethyl-4-oxo-4,5,6,7- tetrahydro-1H-indol-1-yl)pyrazine-2- carboxamide |
| 7 | 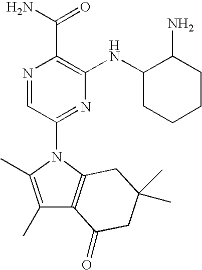 | 3-(2-aminocyclohexylamino)-5- (2,3,6,6-tetramethyl-4-oxo-4,5,6,7- tetrahydro-1H-indol-1-yl)pyrazine-2- carboxamide |
| 8 | 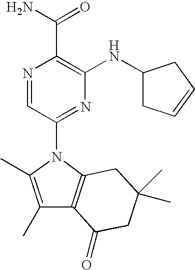 | 3-(cyclopent-3-enylamino)-5-(2,3,6,6- tetramethyl-4-oxo-4,5,6,7-tetrahydro- 1H-indol-1-yl)pyrazine-2-carboxamide |
| 9 | 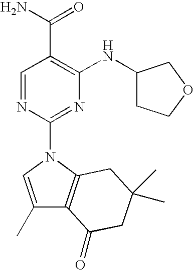 | 4-(tetrahydrofuran-3-ylamino)-2- (3,6,6-trimethyl-4-oxo-4,5,6,7- tetrahydro-1H-indol-1-yl)pyrimidine- 5-carboxamide |
| 10 | 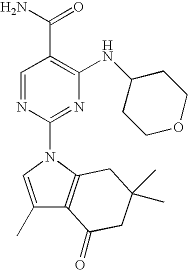 | 4-(tetrahydro-2H-pyran-4-ylamino)-2- (3,6,6-trimethyl-4-oxo-4,5,6,7- tetrahydro-1H-indol-1-yl)pyrimidine- 5-carboxamide |
| 11 | 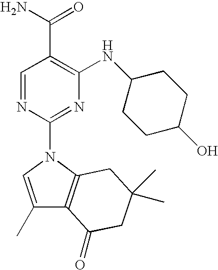 | 4-(4-hydroxycyclohexylamino)-2- (3,6,6-trimethyl-4-oxo-4,5,6,7- tetrahydro-1H-indol-1-yl)pyrimidine- 5-carboxamide |
| 12 | 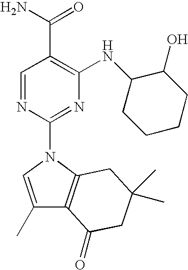 | 4-(2-hydroxycyclohexylamino)-2- (3,6,6-trimethyl-4-oxo-4,5,6,7- tetrahydro-1H-indol-1-yl)pyrimidine- 5-carboxamide |
| 13 | 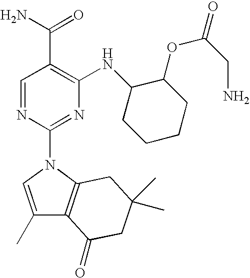 | 2-(5-carbamoyl-2-(3,6,6-trimethyl-4- oxo-4,5,6,7-tetrahydro-1H-indol-1- yl)pyrimidin-4-ylamino)cyclohexyl 2- aminoacetate |
| 14 | 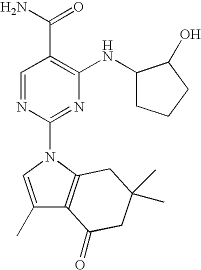 | 4-(2-hydroxycyclopentylamino)-2- (3,6,6-trimethyl-4-oxo-4,5,6,7- tetrahydro-1H-indol-1-yl)pyrimidine- 5-carboxamide |
| 15 | 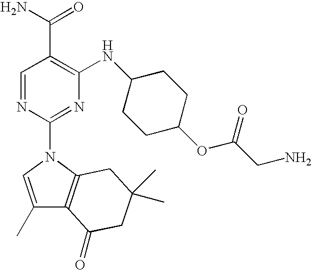 | 4-(5-carbamoyl-2-(3,6,6-trimethyl-4- oxo-4,5,6,7-tetrahydro-1H-indol-1- yl)pyrimidin-4-ylamino)cyclohexyl 2- aminoacetate |
| 16 | 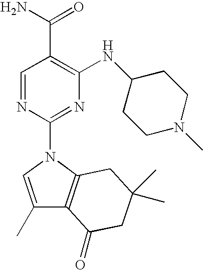 | 4-(1-methylpiperidin-4-ylamino)-2- (3,6,6-trimethyl-4-oxo-4,5,6,7- tetrahydro-1H-indol-1-yl)pyrimidine- 5-carboxamide |
| 17 | 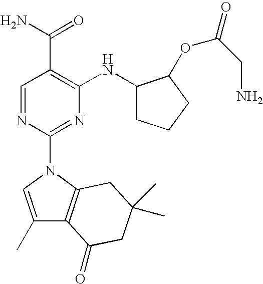 | 2-(5-carbamoyl-2-(3,6,6-trimethyl-4- oxo-4,5,6,7-tetrahydro-1H-indol-1- yl)pyrimidin-4-ylamino)cyclopentyl 2- aminoacetate |
| 18 | 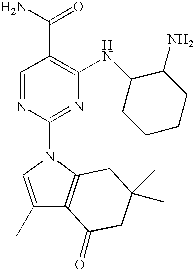 | 4-(2-aminocyclohexylamino)-2-(3,6,6- trimethyl-4-oxo-4,5,6,7-tetrahydro- 1H-indol-1-yl)pyrimidine-5- carboxamide |
| 19 | 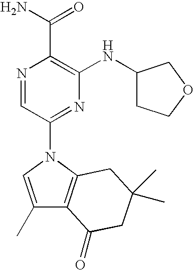 | 3-(tetrahydrofuran-3-ylamino)-5- (3,6,6-trimethyl-4-oxo-4,5,6,7- tetrahydro-1H-indol-1-yl)pyrazine-2- carboxamide |
| 20 | 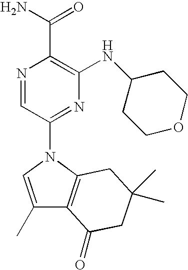 | 3-(tetrahydro-2H-pyran-4-ylamino)-5- (3,6,6-trimethyl-4-oxo-4,5,6,7- tetrahydro-1H-indol-1-yl)pyrazine-2- carboxamide |
| 21 | 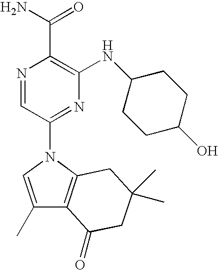 | 3-(4-hydroxycyclohexylamino)-5- (3,6,6-trimethyl-4-oxo-4,5,6,7- tetrahydro-1H-indol-1-yl)pyrazine-2- carboxamide |
| 22 | 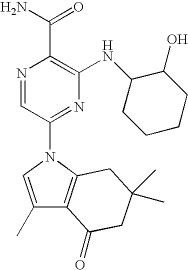 | 3-(2-hydroxycyclohexylamino)-5- (3,6,6-trimethyl-4-oxo-4,5,6,7- tetrahydro-1H-indol-1-yl)pyrazine-2- carboxamide |
| 23 | 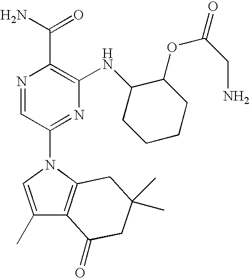 | 2-(3-carbamoyl-6-(3,6,6-trimethyl-4- oxo-4,5,6,7-tetrahydro-1H-indol-1- yl)pyrazin-2-ylamino)cyclohexyl 2- aminoacetate |
| 24 | 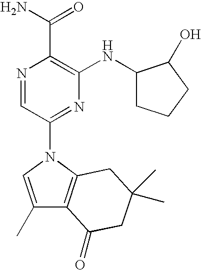 | 3-(2-hydroxycyclopentylamino)-5- (3,6,6-trimethyl-4-oxo-4,5,6,7- tetrahydro-1H-indol-1-yl)pyrazine-2- carboxamide |
| 25 | 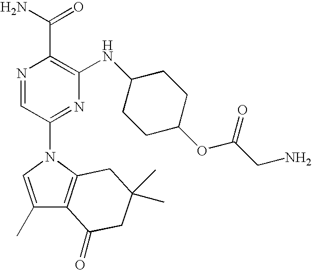 | 4-(3-carbamoyl-6-(3,6,6-trimethyl-4- oxo-4,5,6,7-tetrahydro-1H-indol-1- yl)pyrazin-2-ylamino)cyclohexyl 2- aminoacetate |
| 26 | 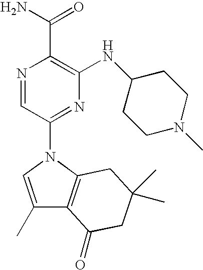 | 3-(1-methylpiperidin-4-ylamino)-5- (3,6,6-trimethyl-4-oxo-4,5,6,7- tetrahydro-1H-indol-1-yl)pyrazine-2- carboxamide |
| 27 | 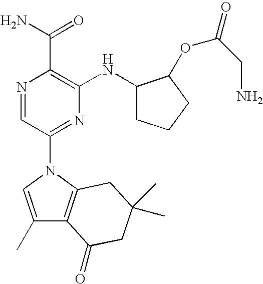 | 2-(3-carbamoyl-6-(3,6,6-trimethyl-4- oxo-4,5,6,7-tetrahydro-1H-indol-1- yl)pyrazin-2-ylamino)cyclopentyl 2- aminoacetate |
| 28 | 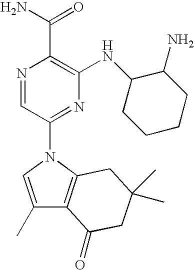 | 3-(2-aminocyclohexylamino)-5-(3,6,6- trimethyl-4-oxo-4,5,6,7-tetrahydro- 1H-indol-1-yl)pyrazine-2-carboxamide |
| 29 | 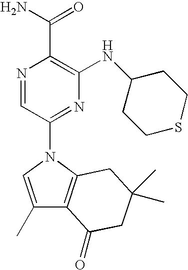 | 3-(tetrahydro-2H-thiopyran-4- ylamino)-5-(3,6,6-trimethyl-4-oxo- 4,5,6,7-tetrahydro-1H-indol-1- yl)pyrazine-2-carboxamide |
| 30 | 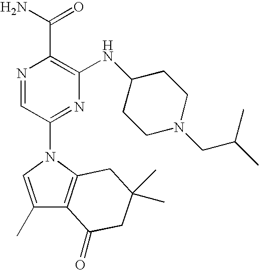 | 3-(1-isobutylpiperidin-4-ylamino)-5- (3,6,6-trimethyl-4-oxo-4,5,6,7- tetrahydro-1H-indol-1-yl)pyrazine-2- carboxamide |
| 31 | 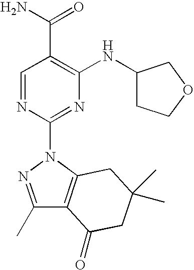 | 4-(tetrahydrofuran-3-ylamino)-2- (3,6,6-trimethyl-4-oxo-4,5,6,7- tetrahydro-1H-indazol-1- yl)pyrimidine-5-carboxamide |
| 32 | 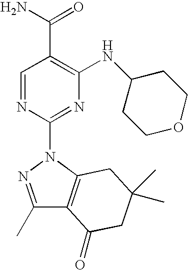 | 4-(tetrahydro-2H-pyran-4-ylamino)-2- (3,6,6-trimethyl-4-oxo-4,5,6,7- tetrahydro-1H-indazol-1- yl)pyrimidine-5-carboxamide |
| 33 | 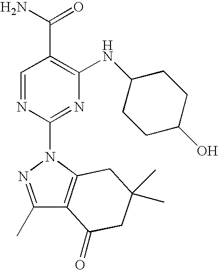 | 4-(4-hydroxycyclohexylamino)-2- (3,6,6-trimethyl-4-oxo-4,5,6,7- tetrahydro-1H-indazol-1- yl)pyrimidine-5-carboxamide |
| 34 | 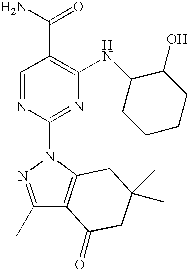 | 4-(2-hydroxycyclohexylamino)-2- (3,6,6-trimethyl-4-oxo-4,5,6,7- tetrahydro-1H-indazol-1- yl)pyrimidine-5-carboxamide |
| 35 | 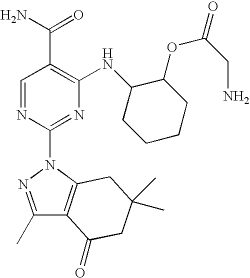 | 2-(5-carbamoyl-2-(3,6,6-trimethyl-4- oxo-4,5,6,7-tetrahydro-1H-indazol-1- yl)pyrimidin-4-ylamino)cyclohexyl 2- aminoacetate |
| 36 | 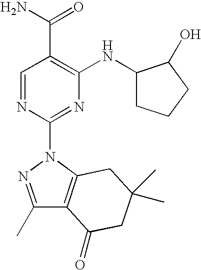 | 4-(2-hydroxycyclopentylamino)-2- (3,6,6-trimethyl-4-oxo-4,5,6,7- tetrahydroindazol-1-yl)pyrimidine-5- carboxamide |
| 37 | 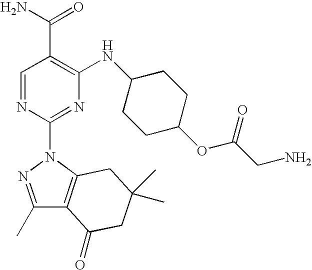 | 4-(5-carbamoyl-2-(3,6,6-trimethyl-4- oxo-4,5,6,7-tetrahydro-1H-indazol-1- yl)pyrimidin-4-ylamino)cyclohexyl 2- aminoacetate |
| 38 | 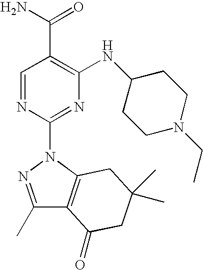 | 4-(1-ethylpiperidin-4-ylamino)-2- (3,6,6-trimethyl-4-oxo-4,5,6,7- tetrahydro-1H-indazol-1- yl)pyrimidine-5-carboxamide |
| 39 | 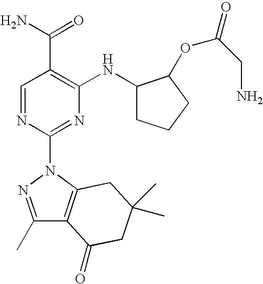 | 2-(5-carbamoyl-2-(3,6,6-trimethyl-4- oxo-4,5,6,7-tetrahydro-1H-indazol-1- yl)pyrimidin-4-ylamino)cyclopentyl 2- aminoacetate |
| 40 | 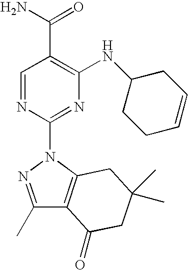 | 4-(cyclohex-3-enylamino)-2-(3,6,6- trimethyl-4-oxo-4,5,6,7-tetrahydro- 1H-indazol-1-yl)pyrimidine-5- carboxamide |
| 41 | 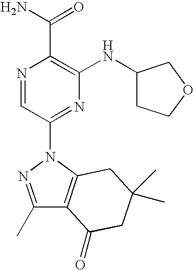 | 3-(tetrahydrofuran-3-ylamino)-5- (3,6,6-trimethyl-4-oxo-4,5,6,7- tetrahydro-1H-indazol-1-yl)pyrazine- 2-carboxamide |
| 42 | 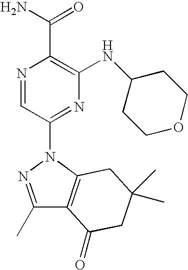 | 3-(tetrahydro-2H-pyran-4-ylamino)-5- (3,6,6-trimethyl-4-oxo-4,5,6,7- tetrahydro-1H-indazol-1-yl)pyrazine- 2-carboxamide |
| 43 | 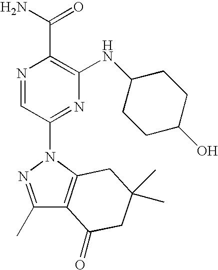 | 3-(4-hydroxycyclohexylamino)-5- (3,6,6-trimethyl-4-oxo-4,5,6,7- tetrahydro-1H-indazol-1-yl)pyrazine- 2-carboxamide |
| 44 | 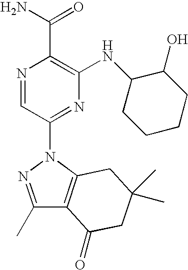 | 3-(2-hydroxycyclohexylamino)-5- (3,6,6-trimethyl-4-oxo-4,5,6,7- tetrahydro-1H-indazol-1-yl)pyrazine- 2-carboxamide |
| 45 | 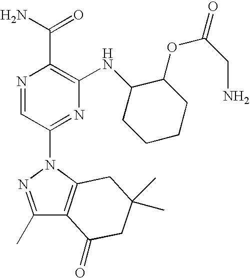 | 2-(3-carbamoyl-6-(3,6,6-trimethyl-4- oxo-4,5,6,7-tetrahydro-1H-indazol-1- yl)pyrazin-2-ylamino)cyclohexyl 2- aminoacetate |
| 46 | 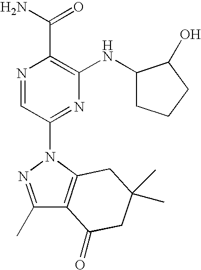 | 3-(2-hydroxycyclopentylamino)-5- (3,6,6-trimethyl-4-oxo-4,5,6,7- tetrahydro-1H-indazol-1-yl)pyrazine- 2-carboxamide |
| 47 | 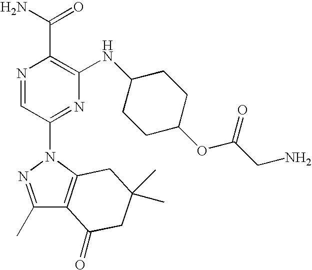 | 4-(3-carbamoyl-6-(3,6,6-trimethyl-4- oxo-4,5,6,7-tetrahydro-1H-indazol-1- yl)pyrazin-2-ylamino)cyclohexyl 2- aminoacetate |
| 48 | 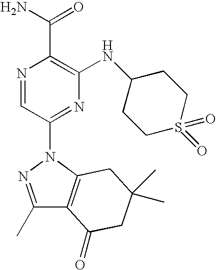 | 3-[(1,1-dioxidotetrahydro-2H- thiopyran-4-yl)amino]-5-(3,6,6- trimethyl-4-oxo-4,5,6,7-tetrahydro- 1H-indazol-1-yl)pyrazine-2- carboxamide |
| 49 | 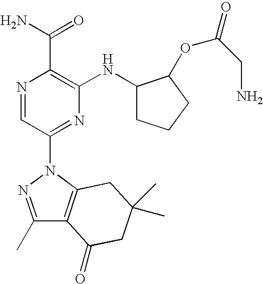 | 2-(3-carbamoyl-6-(3,6,6-trimethyl-4- oxo-4,5,6,7-tetrahydro-1H-indazol-1- yl)pyrazin-2-ylamino)cyclopentyl 2- aminoacetate |
| 50 | 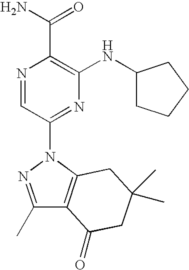 | 3-(cyclopentylamino)-5-(3,6,6- trimethyl-4-oxo-4,5,6,7-tetrahydro- 1H-indazol-1-yl)pyrazine-2- carboxamide |
| 51 | 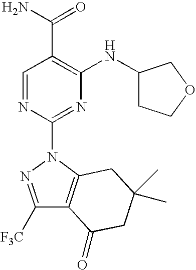 | 2-(6,6-dimethyl-4-oxo-3- (trifluoromethyl)-4,5,6,7-tetrahydro- 1H-indazol-1-yl)-4-(tetrahydrofuran- 3-ylamino)pyrimidine-5-carboxamide |
| 52 | 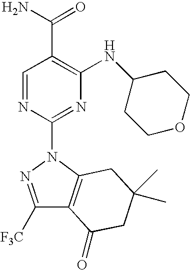 | 2-(6,6-dimethyl-4-oxo-3- (trifluoromethyl)-4,5,6,7-tetrahydro- 1H-indazol-1-yl)-4-(tetrahydro-2H- pyran-4-ylamino)pyrimidine-5- carboxamide |
| 53 | 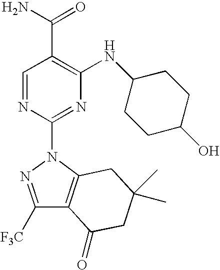 | 2-(6,6-dimethyl-4-oxo-3- (trifluoromethyl)-4,5,6,7-tetrahydro- 1H-indazol-1-yl)-4-(4- hydroxycyclohexylamino)pyrimidine-5- carboxamide |
| 54 | 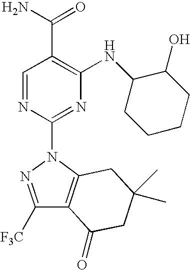 | 2-(6,6-dimethyl-4-oxo-3- (trifluoromethyl)-4,5,6,7-tetrahydro- 1H-indazol-1-yl)-4-(2- hydroxycyclohexylamino)pyrimidine-5- carboxamide |
| 55 | 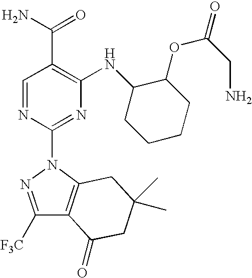 | 2-(5-carbamoyl-2-(6,6-dimethyl-4-oxo- 3-(trifluoromethyl)-4,5,6,7- tetrahydro-1H-indazol-1-yl)pyrimidin- 4-ylamino)cyclohexyl 2-aminoacetate |
| 56 | 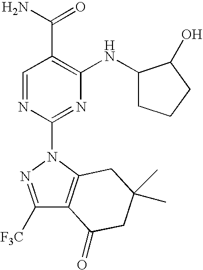 | 2-(6,6-dimethyl-4-oxo-3- (trifluoromethyl)-4,5,6,7-tetrahydro- 1H-indazol-1-yl)-4-(2- hydroxycyclopentylamino)pyrimidine-5- carboxamide |
| 57 | 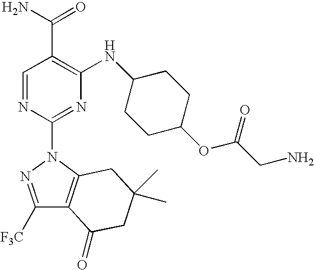 | 4-(5-carbamoyl-2-(6,6-dimethyl-4-oxo- 3-(trifluoromethyl)-4,5,6,7- tetrahydro-1H-indazol-1-yl)pyrimidin- 4-ylamino)cyclohexyl 2-aminoacetate |
| 58 | 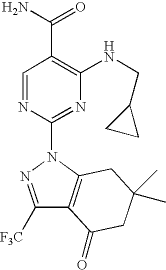 | 4-(cyclopropylmethylamino)-2-(6,6- dimethyl-4-oxo-3-(trifluoromethyl)- 4,5,6,7-tetrahydro-1H-indazol-1- yl)pyrimidine-5-carboxamide |
| 59 | 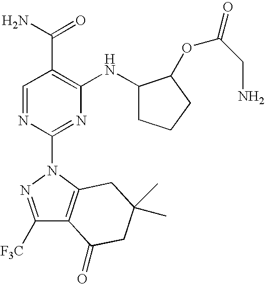 | 2-(5-carbamoyl-2-(6,6-dimethyl-4-oxo- 3-(trifluoromethyl)-4,5,6,7- tetrahydro-1H-indazol-1-yl)pyrimidin- 4-ylamino)cyclopentyl 2-aminoacetate |
| 60 | 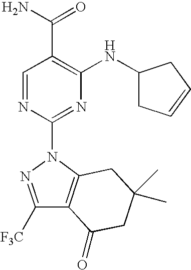 | 4-(cyclopent-3-enylamino)-2-(6,6- dimethyl-4-oxo-3-(trifluoromethyl)- 4,5,6,7-tetrahydro-1H-indazol-1- yl)pyrimidine-5-carboxamide |
| 61 | 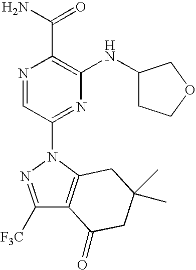 | 5-(6,6-dimethyl-4-oxo-3- (trifluoromethyl)-4,5,6,7-tetrahydro- 1H-indazol-1-yl)-3-(tetrahydrofuran- 3-ylamino)pyrazine-2-carboxamide |
| 62 | 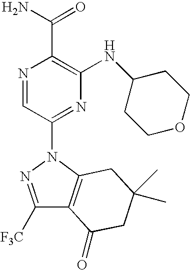 | 5-(6,6-dimethyl-4-oxo-3- (trifluoromethyl)-4,5,6,7-tetrahydro- 1H-indazol-1-yl)-3-(tetrahydro-2H- pyran-4-ylamino)pyrazine-2- carboxamide |
| 63 | 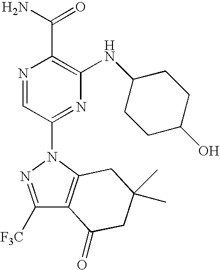 | 5-(6,6-dimethyl-4-oxo-3- (trifluoromethyl)-4,5,6,7-tetrahydro- 1H-indazol-1-yl)-3-(4- hydroxycyclohexylamino)pyrazine-2- carboxamide |
| 64 | 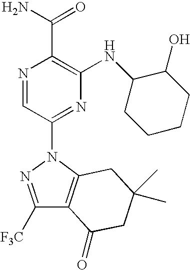 | 5-(6,6-dimethyl-4-oxo-3- (trifluoromethyl)-4,5,6,7-tetrahydro- 1H-indazol-1-yl)-3-(2- hydroxycyclohexylamino)pyrazine-2- carboxamide |
| 65 | 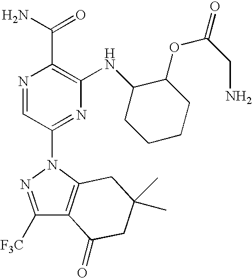 | 2-(3-carbamoyl-6-(6,6-dimethyl-4-oxo- 3-(trifluoromethyl)-4,5,6,7- tetrahydro-1H-indazol-1-yl)pyrazin-2- ylamino)cyclohexyl 2-aminoacetate |
| 66 | 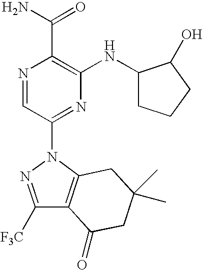 | 5-(6,6-dimethyl-4-oxo-3- (trifluoromethyl)-4,5,6,7-tetrahydro- 1H-indazol-1-yl)-3-(2- hydroxycyclopentylamino)pyrazine-2- carboxamide |
| 67 | 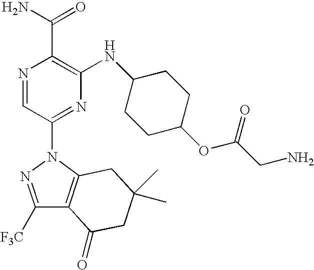 | 4-(3-carbamoyl-6-(6,6-dimethyl-4-oxo- 3-(trifluoromethyl)-4,5,6,7- tetrahydro-1H-indazol-1-yl)pyrazin-2- ylamino)cyclohexyl 2-aminoacetate |
| 68 | 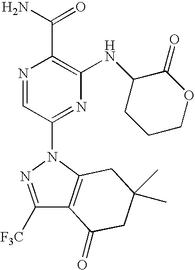 | 5-(6,6-dimethyl-4-oxo-3- (trifluoromethyl)-4,5,6,7-tetrahydro- 1H-indazol-1-yl)-3-(2-oxotetrahydro- 2H-pyran-3-ylamino)pyrazine-2- carboxamide |
| 69 | 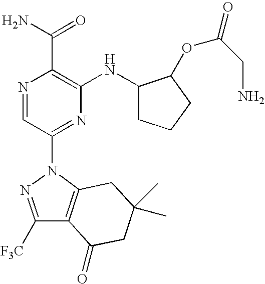 | 2-(3-carbamoyl-6-(6,6-dimethyl-4-oxo- 3-(trifluoromethyl)-4,5,6,7- tetrahydro-1H-indazol-1-yl)pyrazin-2- ylamino)cyclopentyl 2-aminoacetate |
| 70 | 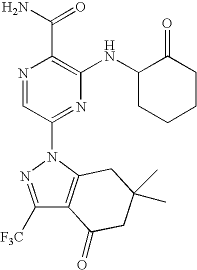 | 5-(6,6-dimethyl-4-oxo-3- (trifluoromethyl)-4,5,6,7-tetrahydro- 1H-indazol-1-yl)-3-(2- oxocyclohexylamino)pyrazine-2- carboxamide |
| 71 | 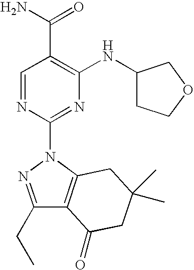 | 2-(3-ethyl-6,6-dimethyl-4-oxo- 4,5,6,7-tetrahydro-1H-indazol-1-yl)- 4-(tetrahydrofuran-3- ylamino)pyrimidine-5-carboxamide |
| 72 | 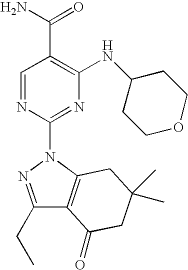 | 2-(3-ethyl-6,6-dimethyl-4-oxo- 4,5,6,7-tetrahydro-1H-indazol-1-yl)- 4-(tetrahydro-2H-pyran-4- ylamino)pyrimidine-5-carboxamide |
| 73 | 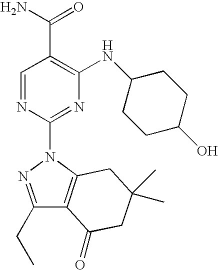 | 2-(3-ethyl-6,6-dimethyl-4-oxo- 4,5,6,7-tetrahydro-1H-indazol-1-yl)- 4-(4- hydroxycyclohexylamino)pyrimidine-5- carboxamide |
| 74 | 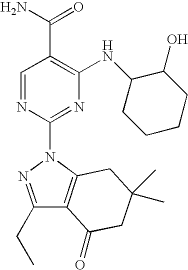 | 2-(3-ethyl-6,6-dimethyl-4-oxo- 4,5,6,7-tetrahydro-1H-indazol-1-yl)- 4-(2- hydroxycyclohexylamino)pyrimidine-5- carboxamide |
| 75 | 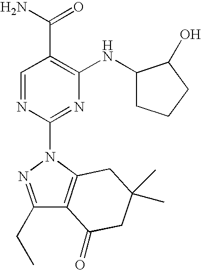 | 2-(3-ethyl-6,6-dimethyl-4-oxo- 4,5,6,7-tetrahydro-1H-indazol-1-yl)- 4-(2- hydroxycyclopentylamino)pyrimidine-5- carboxamide |
| 76 | 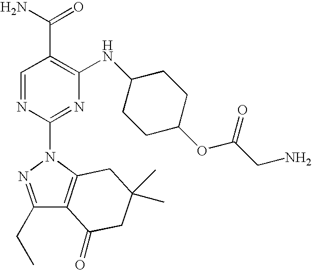 | 4-(5-carbamoyl-2-(3-ethyl-6,6- dimethyl-4-oxo-4,5,6,7-tetrahydro-1H- indazol-1-yl)pyrimidin-4- ylamino)cyclohexyl 2-aminoacetate |
| 77 | 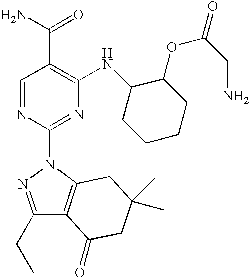 | 2-(5-carbamoyl-2-(3-ethyl-6,6- dimethyl-4-oxo-4,5,6,7-tetrahydro-1H- indazol-1-yl)pyrimidin-4- ylamino)cyclohexyl 2-aminoacetate |
| 78 | 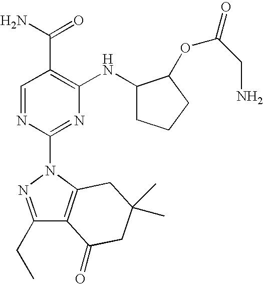 | 2-(5-carbamoyl-2-(3-ethyl-6,6- dimethyl-4-oxo-4,5,6,7-tetrahydro-1H- indazol-1-yl)pyrimidin-4- ylamino)cyclopentyl 2-aminoacetate |
| 79 | 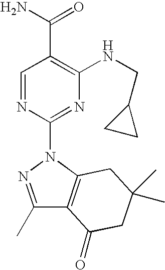 | 4-(cyclopropylmethylamino)-2-(3,6,6- trimethyl-4-oxo-4,5,6,7-tetrahydro- 1H-indazol-1-yl)pyrimidine-5- carboxamide |
| 80 | 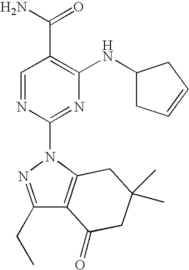 | 4-(cyclopent-3-enylamino)-2-(3-ethyl- 6,6-dimethyl-4-oxo-4,5,6,7- tetrahydro-1H-indazol-1- yl)pyrimidine-5-carboxamide |
| 81 | 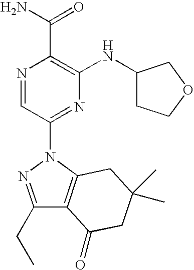 | 5-(3-ethyl-6,6-dimethyl-4-oxo- 4,5,6,7-tetrahydro-1H-indazol-1-yl)- 3-(tetrahydrofuran-3- ylamino)pyrazine-2-carboxamide |
| 82 | 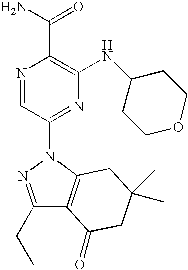 | 5-(3-ethyl-6,6-dimethyl-4-oxo- 4,5,6,7-tetrahydro-1H-indazol-1-yl)- 3-(tetrahydro-2H-pyran-4- ylamino)pyrazine-2-carboxamide |
| 83 | 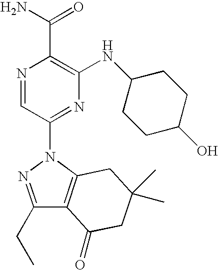 | 5-(3-ethyl-6,6-dimethyl-4-oxo- 4,5,6,7-tetrahydro-1H-indazol-1-yl)- 3-(4-hydroxycyclohexylamino)pyrazine- 2-carboxamide |
| 84 | 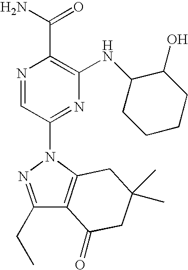 | 5-(3-ethyl-6,6-dimethyl-4-oxo- 4,5,6,7-tetrahydro-1H-indazol-1-yl)- 3-(2-hydroxycyclohexylamino)pyrazine- 2-carboxamide |
| 85 | 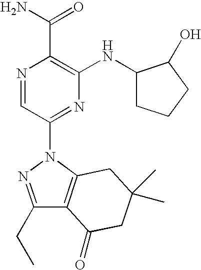 | 5-(3-ethyl-6,6-dimethyl-4-oxo- 4,5,6,7-tetrahydro-1H-indazol-1-yl)- 3-(2- hydroxycyclopentylamino)pyrazine-2- carboxamide |
| 86 | 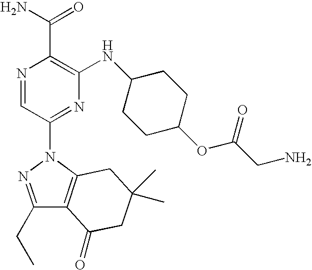 | 4-(3-carbamoyl-6-(3-ethyl-6,6- dimethyl-4-oxo-4,5,6,7-tetrahydro-1H- indazol-1-yl)pyrazin-2- ylamino)cyclohexyl 2-aminoacetate |
| 87 | 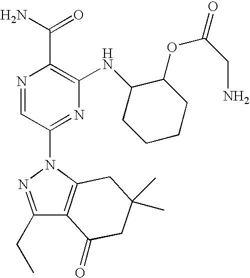 | 2-(3-carbamoyl-6-(3-ethyl-6,6- dimethyl-4-oxo-4,5,6,7-tetrahydro-1H- indazol-1-yl)pyrazin-2- ylamino)cyclohexyl 2-aminoacetate |
| 88 | 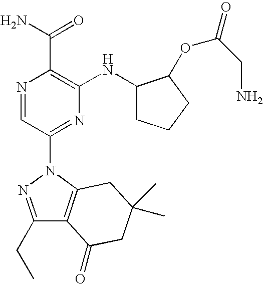 | 2-(3-carbamoyl-6-(3-ethyl-6,6- dimethyl-4-oxo-4,5,6,7-tetrahydro-1H- indazol-1-yl)pyrazin-2- ylamino)cyclopentyl 2-aminoacetate |
| 89 | 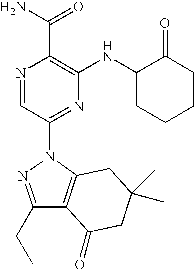 | 5-(3-ethyl-6,6-dimethyl-4-oxo- 4,5,6,7-tetrahydro-1H-indazol-1-yl)- 3-(2-oxocyclohexylamino)pyrazine-2- carboxamide |
| 90 | 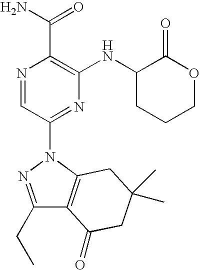 | 5-(3-ethyl-6,6-dimethyl-4-oxo- 4,5,6,7-tetrahydro-1H-indazol-1-yl)- 3-(2-oxotetrahydro-2H-pyran-3- ylamino)pyrazine-2-carboxamide |
| 91 | 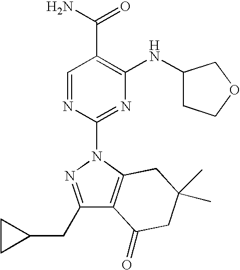 | 2-(3-(cyclopropylmethyl)-6,6- dimethyl-4-oxo-4,5,6,7-tetrahydro-1H- indazol-1-yl)-4-(tetrahydrofuran-3- ylamino)pyrimidine-5-carboxamide |
| 92 | 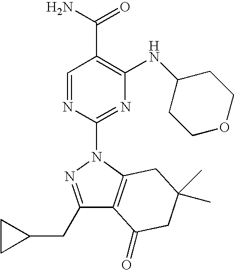 | 2-(3-(cyclopropylmethyl)-6,6- dimethyl-4-oxo-4,5,6,7-tetrahydro-1H- indazol-1-yl)-4-(tetrahydro-2H-pyran- 4-ylamino)pyrimidine-5-carboxamide |
| 93 | 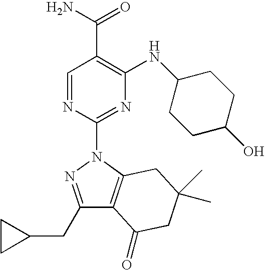 | 2-(3-(cyclopropylmethyl)-6,6- dimethyl-4-oxo-4,5,6,7-tetrahydro-1H- indazol-1-yl)-4-(4- hydroxycyclohexylamino)pyrimidine-5- carboxamide |
| 94 | 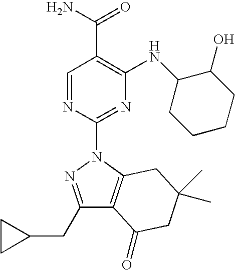 | 2-(3-(cyclopropylmethyl)-6,6- dimethyl-4-oxo-4,5,6,7-tetrahydro-1H- indazol-1-yl)-4-(2- hydroxycyclohexylamino)pyrimidine-5- carboxamide |
| 95 | 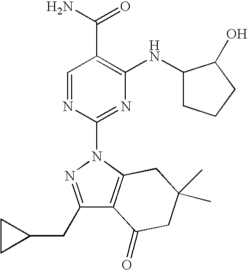 | 2-(3-(cyclopropylmethyl)-6,6- dimethyl-4-oxo-4,5,6,7-tetrahydro-1H- indazol-1-yl)-4-(2- hydroxycyclopentylamino)pyrimidine-5- carboxamide |
| 96 | 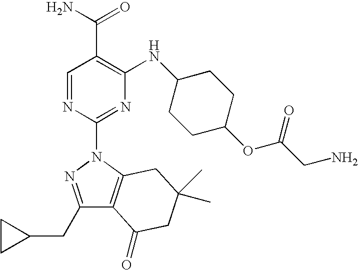 | 4-(5-carbamoyl-2-(3- (cyclopropylmethyl)-6,6-dimethyl-4- oxo-4,5,6,7-tetrahydro-1H-indazol-1- yl)pyrimidin-4-ylamino)cyclohexyl 2- aminoacetate |
| 97 | 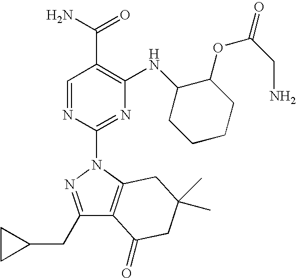 | 2-(5-carbamoyl-2-(3- (cyclopropylmethyl)-6,6-dimethyl-4- oxo-4,5,6,7-tetrahydro-1H-indazol-1- yl)pyrimidin-4-ylamino)cyclohexyl 2- aminoacetate |
| 98 | 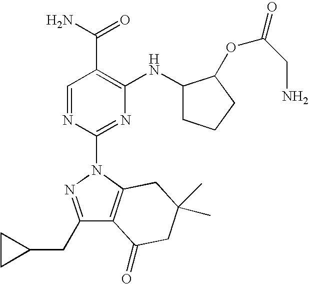 | 2-(5-carbamoyl-2-(3- (cyclopropylmethyl)-6,6-dimethyl-4- oxo-4,5,6,7-tetrahydro-1H-indazol-1- yl)pyrimidin-4-ylamino)cyclopentyl 2- aminoacetate |
| 99 | 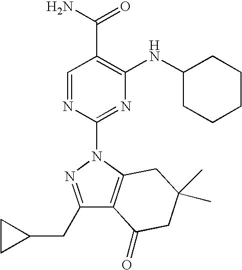 | 4-(cyclohexylamino)-2-(3- (cyclopropylmethyl)-6,6-dimethyl-4- oxo-4,5,6,7-tetrahydro-1H-indazol-1- yl)pyrimidine-5-carboxamide |
| 100 | 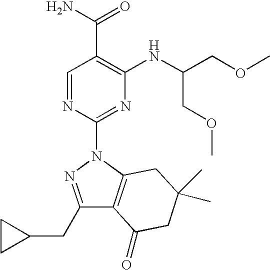 | 2-(3-(cyclopropylmethyl)-6,6- dimethyl-4-oxo-4,5,6,7-tetrahydro-1H- indazol-1-yl)-4-(1,3-dimethoxypropan- 2-ylamino)pyrimidine-5-carboxamide |
| 101 | 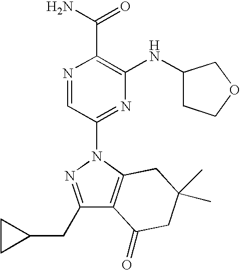 | 5-(3-(cyclopropylmethyl)-6,6- dimethyl-4-oxo-4,5,6,7-tetrahydro-1H- indazol-1-yl)-3-(tetrahydrofuran-3- ylamino)pyrazine-2-carboxamide |
| 102 | 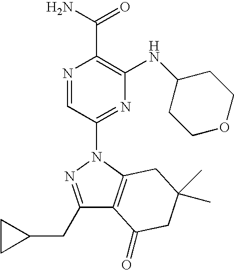 | 5-(3-(cyclopropylmethyl)-6,6- dimethyl-4-oxo-4,5,6,7-tetrahydro-1H- indazol-1-yl)-3-(tetrahydro-2H-pyran- 4-ylamino)pyrazine-2-carboxamide |
| 103 | 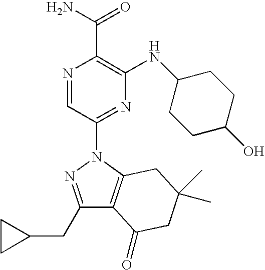 | 5-(3-(cyclopropylmethyl)-6,6- dimethyl-4-oxo-4,5,6,7-tetrahydro-1H- indazol-1-yl)-3-(4- hydroxycyclohexylamino)pyrazine-2- carboxamide |
| 104 | 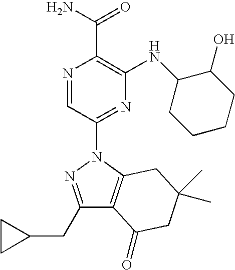 | 5-(3-(cyclopropylmethyl)-6,6- dimethyl-4-oxo-4,5,6,7-tetrahydro-1H- indazol-1-yl)-3-(2- hydroxycyclohexylamino)pyrazine-2- carboxamide |
| 105 | 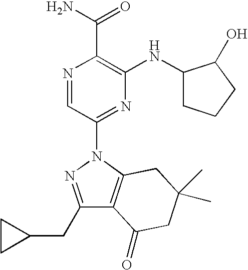 | 5-(3-(cyclopropylmethyl)-6,6- dimethyl-4-oxo-4,5,6,7-tetrahydro-1H- indazol-1-yl)-3-(2- hydroxycyclopentylamino)pyrazine-2- carboxamide |
| 106 | 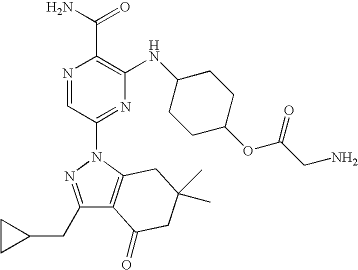 | 4-(3-carbamoyl-6-(3- (cyclopropylmethyl)-6,6-dimethyl-4- oxo-4,5,6,7-tetrahydro-1H-indazol-1- yl)pyrazin-2-ylamino)cyclohexyl 2- aminoacetate |
| 107 | 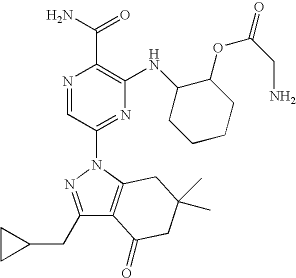 | 2-(3-carbamoyl-6-(3- (cyclopropylmethyl)-6,6-dimethyl-4- oxo-4,5,6,7-tetrahydro-1H-indazol-1- yl)pyrazin-2-ylamino)cyclohexyl 2- aminoacetate |
| 108 | 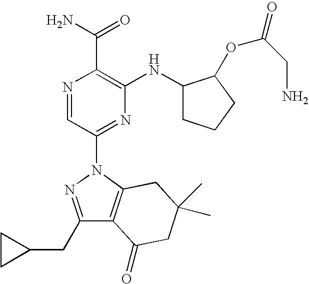 | 2-(3-carbamoyl-6-(3- (cyclopropylmethyl)-6,6-dimethyl-4- oxo-4,5,6,7-tetrahydro-1H-indazol-1- yl)pyrazin-2-ylamino)cyclopentyl 2- aminoacetate |
| 109 | 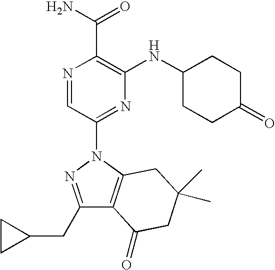 | 5-(3-(cyclopropylmethyl)-6,6- dimethyl-4-oxo-4,5,6,7-tetrahydro-1H- indazol-1-yl)-3-(4- oxocyclohexylamino)pyrazine-2- carboxamide |
| 110 | 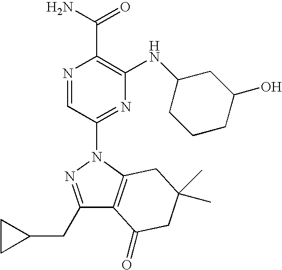 | 5-(3-(cyclopropylmethyl)-6,6- dimethyl-4-oxo-4,5,6,7-tetrahydro-1H- indazol-1-yl)-3-(3- hydroxycyclohexylamino)pyrazine-2- carboxamide |
| 111 | 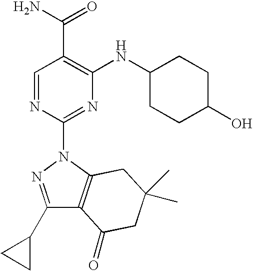 | 2-(3-cyclopropyl-6,6-dimethyl-4-oxo- 4,5,6,7-tetrahydro-1H-indazol-1-yl)- 4-(4- hydroxycyclohexylamino)pyrimidine-5- carboxamide |
| 112 | 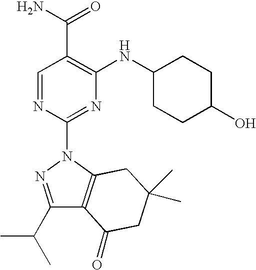 | 4-(4-hydroxycyclohexylamino)-2-(3- isopropyl-6,6-dimethyl-4-oxo-4,5,6,7- tetrahydro-1H-indazol-1- yl)pyrimidine-5-carboxamide |
| 113 | 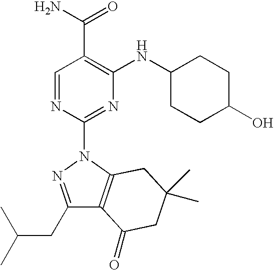 | 4-(4-hydroxycyclohexylamino)-2-(3- isobutyl-6,6-dimethyl-4-oxo-4,5,6,7- tetrahydro-1H-indazol-1- yl)pyrimidine-5-carboxamide |
| 114 | 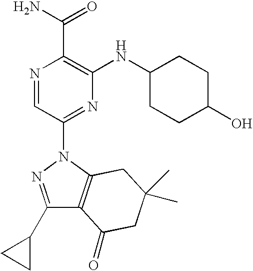 | 5-(3-cyclopropyl-6,6-dimethyl-4-oxo- 4,5,6,7-tetrahydro-1H-indazol-1-yl)- 3-(4-hydroxycyclohexylamino)pyrazine- 2-carboxamide |
| 115 | 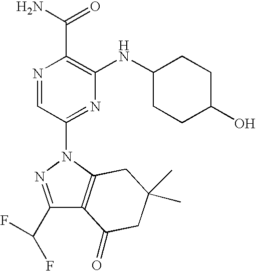 | 5-(3-(difluoromethyl)-6,6-dimethyl-4- oxo-4,5,6,7-tetrahydro-1H-indazol-1- yl)-3-(4- hydroxycyclohexylamino)pyrazine-2- carboxamide |
| 116 | 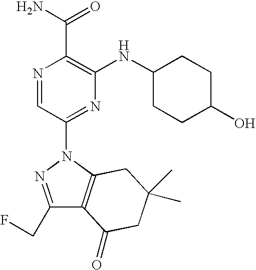 | 5-(3-(fluoromethyl)-6,6-dimethyl-4- oxo-4,5,6,7-tetrahydro-1H-indazol-1- yl)-3-(4- hydroxycyclohexylamino)pyrazine-2- carboxamide |

### Example 4

The compounds listed below in Tables 1-8 are prepared essentially according to the procedures outlined in the above schemes and detailed in the preceding synthetic examples. Thus, the procedures for preparing the following compounds use the same or analogous synthetic techniques with substitution of alternative starting materials as necessary. Suitable variations and alternatives for preparing the following compounds will be readily apparent to those skilled in the art of organic synthesis.

In each of the following tables 1-8, the various substituents are defined in the following table.

```

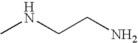

  1

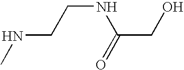

  2

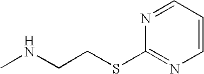

  3

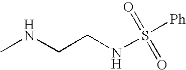

  4

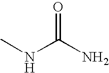

  5

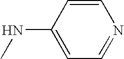

  6

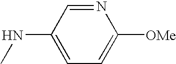

  7

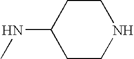

  8

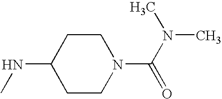

  9
—OEt
  10

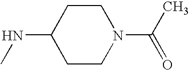

  11

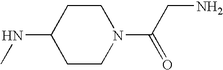

  12

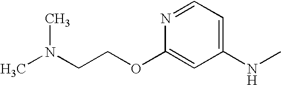

  13

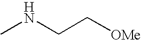

  14

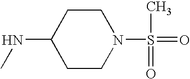

  15

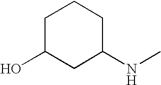

  16

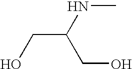

  17

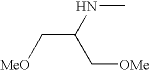

  18

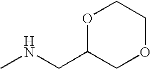

  19

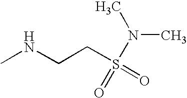

  20

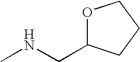

  21

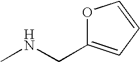

  22

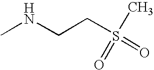

  23

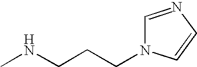

  24

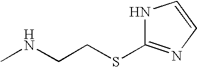

  25

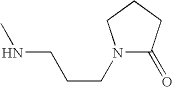

  26

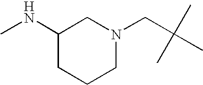

  27

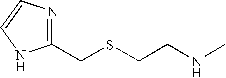

  28

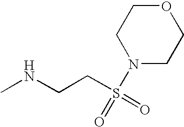

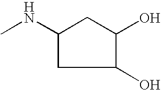

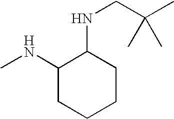



  31

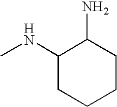

  32

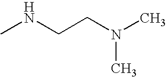

  33

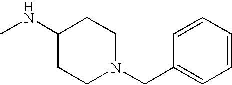

  34

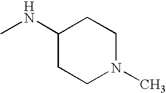

  35

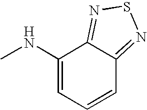

  36

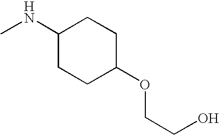

  37

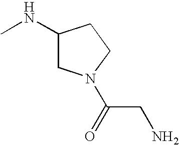

  38

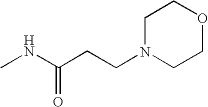

  39

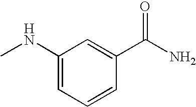

  40

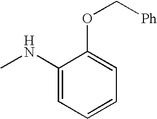

  41

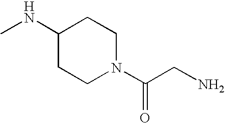

  42

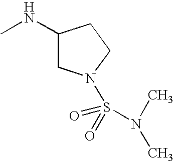

  43

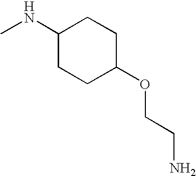

  44

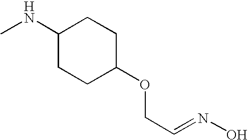

  45

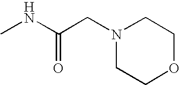

  46

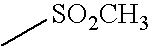

  47

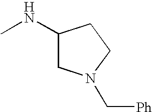

  48

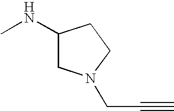

  49

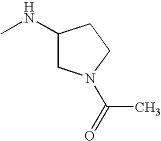

  50

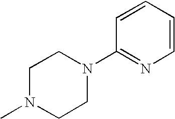

  51

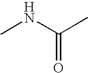

  52

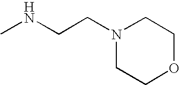

  53

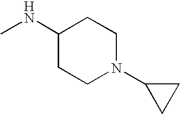

  54

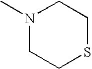

  55

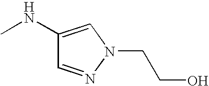

  56

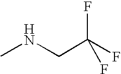

  57

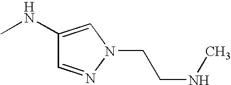

  58

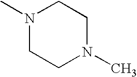

  59

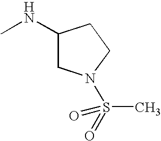

  60

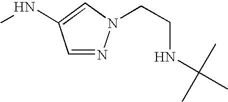

  61

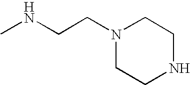

  62

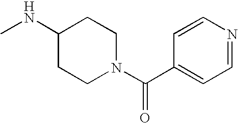

  63

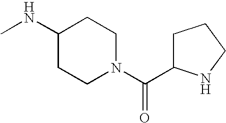

  64

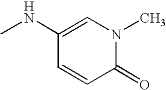

  65

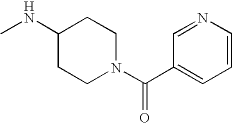

  66

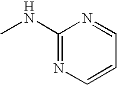

  67

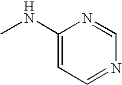

  68

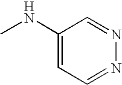

  69

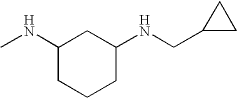

  70

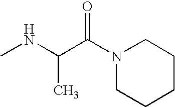

  71

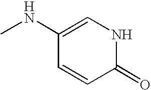

  72

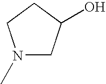

  73

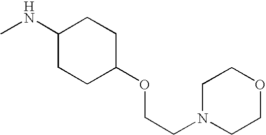

  74

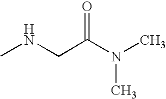

  75

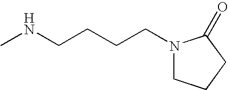

  76

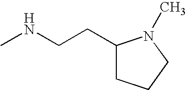

  77

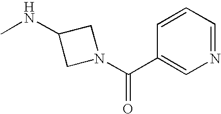

  78

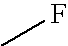

  79

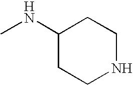

  80

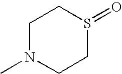

  81

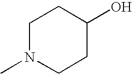

  82

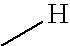

  83

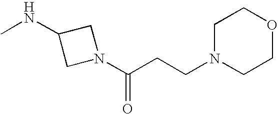

  84

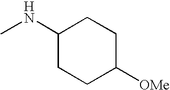

  85

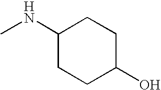

  86

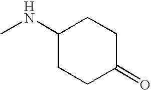

  87

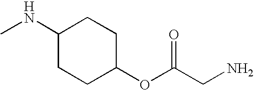

  88

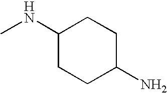

  89

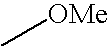

  90

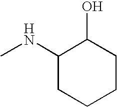

  91

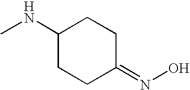

  92

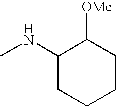

  93

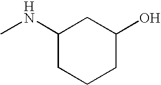

  94

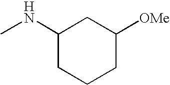

  95

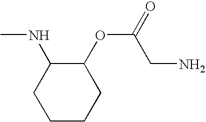

  96

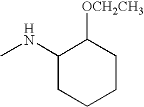

  97

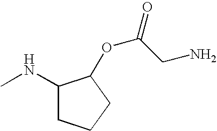

  98

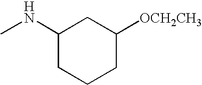

  99

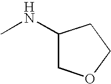

100

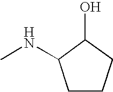

101

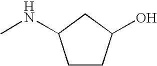

102

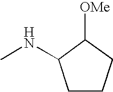

103

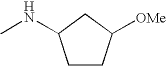

104

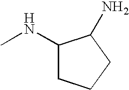

105

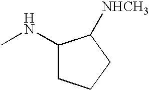

106

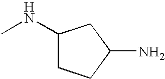

107

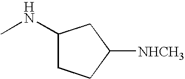

108

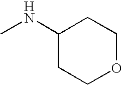

109

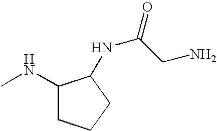

110

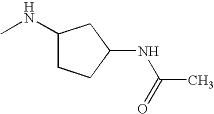

111

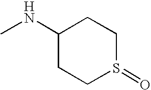

112

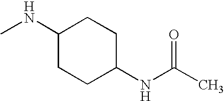

113

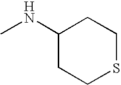

114

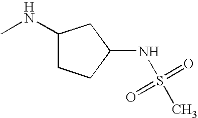

115

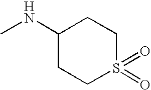

116

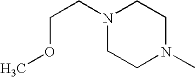

117
—Cl
118

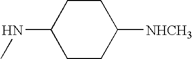

119

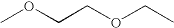

120
—SCH₃
121
—Br
122

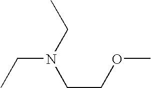

123

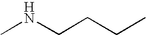

124

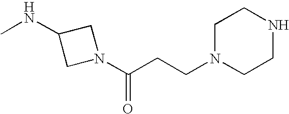

125

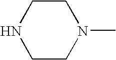

126

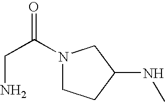

127

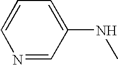

128
—CH₃
129
—CF₃
130
H₃C—
201

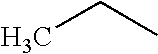

202

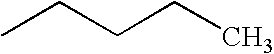

203

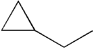

204

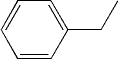

205

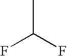

206

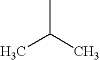

207

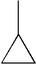

208
F₃C—
209

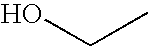

210

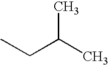

211
—H
212
═O
301

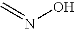

302

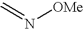

303

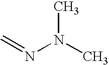

304

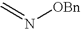

305

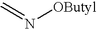

306

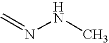

307

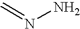

308
```

Compounds having the formula:

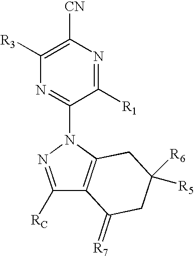

wherein R₁, R₃, R_{C}, R₅, R₆, and R₇ are defined in Table 1:

TABLE 1

| Compound | | | | | | |
|---|---|---|---|---|---|---|
| No. | R₁ | R₃ | R_{c} | R₅ | R₆ | R₇ |
| 117 | 10 | 1 | 201 | 212 | 212 | 308 |
| 118 | 130 | 50 | 212 | 202 | 212 | 307 |
| 119 | 130 | 121 | 209 | 202 | 212 | 308 |
| 120 | 83 | 109 | 207 | 201 | 201 | 302 |
| 121 | 90 | 101 | 211 | 201 | 201 | 306 |
| 122 | 90 | 66 | 201 | 212 | 212 | 304 |
| 123 | 130 | 56 | 209 | 202 | 212 | 304 |
| 124 | 118 | 43 | 212 | 202 | 212 | 308 |
| 125 | 90 | 83 | 211 | 212 | 212 | 303 |
| 126 | 79 | 88 | 206 | 202 | 212 | 303 |
| 127 | 118 | 114 | 212 | 202 | 212 | 304 |
| 128 | 90 | 85 | 204 | 202 | 212 | 301 |
| 129 | 129 | 62 | 205 | 212 | 212 | 303 |
| 130 | 10 | 46 | 204 | 202 | 212 | 308 |
| 131 | 90 | 39 | 204 | 212 | 212 | 302 |
| 132 | 79 | 116 | 205 | 202 | 212 | 301 |
| 133 | 83 | 86 | 201 | 212 | 212 | 302 |
| 134 | 83 | 15 | 211 | 212 | 212 | 302 |
| 135 | 90 | 7 | 204 | 201 | 201 | 307 |
| 136 | 129 | 6 | 210 | 202 | 212 | 306 |
| 137 | 118 | 73 | 210 | 202 | 212 | 308 |
| 138 | 118 | 57 | 202 | 212 | 212 | 308 |
| 139 | 90 | 86 | 210 | 212 | 212 | 303 |
| 140 | 79 | 109 | 204 | 201 | 201 | 303 |
| 141 | 90 | 86 | 204 | 212 | 212 | 304 |
| 142 | 130 | 69 | 210 | 201 | 201 | 303 |
| 143 | 129 | 88 | 208 | 212 | 212 | 301 |
| 144 | 10 | 98 | 208 | 201 | 201 | 308 |
| 145 | 118 | 102 | 201 | 201 | 201 | 308 |
| 146 | 90 | 90 | 205 | 212 | 212 | 305 |
| 147 | 10 | 96 | 209 | 201 | 201 | 302 |
| 148 | 90 | 91 | 210 | 201 | 201 | 308 |
| 149 | 130 | 109 | 212 | 201 | 201 | 305 |
| 150 | 90 | 78 | 204 | 201 | 201 | 307 |
| 151 | 130 | 93 | 205 | 212 | 212 | 307 |
| 152 | 83 | 17 | 207 | 201 | 201 | 308 |
| 153 | 90 | 23 | 205 | 202 | 212 | 301 |
| 154 | 10 | 3 | 203 | 202 | 212 | 302 |
| 155 | 118 | 67 | 211 | 201 | 201 | 302 |
| 156 | 83 | 88 | 206 | 212 | 212 | 301 |
| 157 | 129 | 72 | 204 | 202 | 212 | 305 |
| 158 | 118 | 34 | 205 | 201 | 201 | 304 |
| 159 | 90 | 96 | 201 | 201 | 201 | 304 |
| 160 | 118 | 64 | 212 | 201 | 201 | 308 |
| 161 | 10 | 125 | 201 | 212 | 212 | 308 |
| 162 | 130 | 117 | 211 | 202 | 212 | 302 |
| 163 | 83 | 60 | 202 | 201 | 201 | 301 |
| 164 | 129 | 9 | 212 | 212 | 212 | 305 |
| 165 | 10 | 74 | 210 | 201 | 201 | 303 |
| 166 | 79 | 79 | 206 | 212 | 212 | 301 |
| 167 | 118 | 19 | 202 | 201 | 201 | 308 |
| 168 | 90 | 86 | 205 | 201 | 201 | 303 |
| 169 | 10 | 52 | 201 | 202 | 212 | 306 |
| 170 | 79 | 91 | 212 | 201 | 201 | 304 |
| 171 | 90 | 10 | 211 | 202 | 212 | 308 |
| 172 | 10 | 22 | 211 | 201 | 201 | 308 |
| 173 | 83 | 113 | 210 | 201 | 201 | 307 |
| 174 | 90 | 13 | 204 | 201 | 201 | 302 |
| 175 | 83 | 91 | 206 | 212 | 212 | 307 |
| 176 | 10 | 48 | 212 | 202 | 212 | 303 |
| 177 | 83 | 88 | 209 | 212 | 212 | 307 |
| 178 | 83 | 36 | 212 | 201 | 201 | 301 |
| 179 | 10 | 85 | 204 | 212 | 212 | 308 |
| 180 | 118 | 108 | 205 | 212 | 212 | 302 |
| 181 | 83 | 98 | 209 | 201 | 201 | 307 |
| 182 | 79 | 61 | 202 | 202 | 212 | 301 |
| 183 | 90 | 122 | 201 | 201 | 201 | 308 |
| 184 | 118 | 47 | 203 | 202 | 212 | 306 |
| 185 | 118 | 76 | 202 | 202 | 212 | 306 |
| 186 | 129 | 55 | 204 | 201 | 201 | 302 |
| 187 | 90 | 101 | 201 | 212 | 212 | 308 |
| 188 | 130 | 85 | 210 | 202 | 212 | 303 |
| 189 | 130 | 104 | 210 | 202 | 212 | 307 |
| 190 | 130 | 14 | 203 | 201 | 201 | 308 |
| 191 | 10 | 118 | 203 | 212 | 212 | 306 |
| 192 | 130 | 88 | 204 | 202 | 212 | 306 |
| 193 | 129 | 109 | 207 | 201 | 201 | 304 |
| 194 | 130 | 112 | 212 | 201 | 201 | 304 |
| 195 | 129 | 91 | 201 | 201 | 201 | 305 |
| 196 | 10 | 91 | 203 | 201 | 201 | 306 |
| 197 | 129 | 28 | 212 | 202 | 212 | 302 |
| 198 | 90 | 96 | 212 | 212 | 212 | 307 |
| 199 | 90 | 53 | 204 | 201 | 201 | 303 |
| 200 | 90 | 111 | 204 | 202 | 212 | 308 |
| 201 | 90 | 82 | 209 | 202 | 212 | 302 |
| 202 | 129 | 107 | 204 | 201 | 201 | 302 |
| 203 | 90 | 98 | 204 | 212 | 212 | 301 |
| 204 | 90 | 100 | 212 | 212 | 212 | 308 |
| 205 | 118 | 130 | 206 | 202 | 212 | 306 |
| 206 | 10 | 109 | 201 | 212 | 212 | 301 |
| 207 | 10 | 85 | 212 | 212 | 212 | 307 |
| 208 | 129 | 68 | 204 | 212 | 212 | 304 |
| 209 | 130 | 106 | 205 | 212 | 212 | 307 |
| 210 | 118 | 49 | 209 | 201 | 201 | 307 |
| 211 | 118 | 35 | 209 | 202 | 212 | 301 |
| 212 | 130 | 89 | 206 | 202 | 212 | 306 |
| 213 | 129 | 58 | 202 | 212 | 212 | 306 |
| 214 | 118 | 105 | 212 | 202 | 212 | 306 |
| 215 | 10 | 109 | 207 | 201 | 201 | 306 |
| 216 | 83 | 98 | 208 | 201 | 201 | 301 |
| 217 | 90 | 101 | 207 | 212 | 212 | 301 |
| 218 | 129 | 101 | 212 | 202 | 212 | 307 |
| 219 | 79 | 126 | 212 | 202 | 212 | 307 |
| 220 | 10 | 86 | 212 | 212 | 212 | 301 |
| 221 | 118 | 88 | 210 | 202 | 212 | 306 |
| 222 | 83 | 129 | 211 | 202 | 212 | 302 |
| 223 | 79 | 127 | 210 | 201 | 201 | 301 |
| 224 | 83 | 38 | 206 | 201 | 201 | 305 |
| 225 | 79 | 24 | 204 | 201 | 201 | 301 |
| 226 | 130 | 31 | 208 | 212 | 212 | 305 |
| 227 | 118 | 63 | 210 | 202 | 212 | 308 |
| 228 | 90 | 2 | 201 | 201 | 201 | 308 |
| 229 | 83 | 30 | 210 | 201 | 201 | 305 |
| 230 | 10 | 98 | 205 | 201 | 201 | 302 |
| 231 | 90 | 21 | 212 | 202 | 212 | 301 |
| 232 | 118 | 96 | 201 | 201 | 201 | 308 |
| 233 | 90 | 96 | 211 | 201 | 201 | 301 |
| 234 | 83 | 54 | 212 | 201 | 201 | 302 |
| 235 | 118 | 42 | 205 | 212 | 212 | 308 |
| 236 | 118 | 59 | 206 | 201 | 201 | 306 |
| 237 | 130 | 25 | 204 | 202 | 212 | 306 |
| 238 | 10 | 32 | 211 | 212 | 212 | 301 |
| 239 | 83 | 33 | 203 | 202 | 212 | 302 |
| 240 | 90 | 70 | 205 | 201 | 201 | 302 |
| 241 | 83 | 4 | 201 | 201 | 201 | 304 |
| 242 | 129 | 85 | 204 | 212 | 212 | 301 |
| 243 | 130 | 101 | 205 | 202 | 212 | 302 |
| 244 | 118 | 109 | 204 | 212 | 212 | 303 |
| 245 | 79 | 96 | 212 | 201 | 201 | 301 |
| 246 | 83 | 124 | 203 | 201 | 201 | 308 |
| 247 | 90 | 12 | 207 | 201 | 201 | 303 |
| 248 | 130 | 123 | 201 | 201 | 201 | 307 |
| 249 | 10 | 91 | 209 | 212 | 212 | 302 |
| 250 | 83 | 44 | 202 | 201 | 201 | 307 |
| 251 | 90 | 103 | 205 | 201 | 201 | 301 |
| 252 | 83 | 8 | 204 | 212 | 212 | 301 |
| 253 | 129 | 40 | 210 | 202 | 212 | 306 |
| 254 | 130 | 115 | 206 | 201 | 201 | 308 |
| 255 | 118 | 85 | 211 | 212 | 212 | 301 |
| 256 | 118 | 37 | 204 | 202 | 212 | 306 |
| 257 | 79 | 75 | 204 | 202 | 212 | 302 |
| 258 | 130 | 88 | 211 | 201 | 201 | 306 |
| 259 | 90 | 96 | 204 | 201 | 201 | 308 |
| 260 | 129 | 92 | 204 | 212 | 212 | 301 |
| 261 | 79 | 86 | 201 | 202 | 212 | 301 |
| 262 | 118 | 87 | 206 | 202 | 212 | 302 |
| 263 | 130 | 29 | 210 | 202 | 212 | 306 |
| 264 | 10 | 11 | 212 | 212 | 212 | 306 |
| 265 | 129 | 20 | 201 | 201 | 201 | 305 |
| 266 | 118 | 120 | 201 | 201 | 201 | 304 |
| 267 | 79 | 110 | 207 | 201 | 201 | 302 |
| 268 | 90 | 94 | 201 | 212 | 212 | 304 |
| 269 | 130 | 95 | 204 | 201 | 201 | 308 |
| 270 | 10 | 80 | 204 | 212 | 212 | 306 |
| 271 | 118 | 18 | 202 | 202 | 212 | 306 |
| 272 | 10 | 98 | 212 | 201 | 201 | 307 |
| 273 | 10 | 45 | 205 | 201 | 201 | 303 |
| 274 | 79 | 97 | 210 | 201 | 201 | 304 |
| 275 | 10 | 77 | 204 | 212 | 212 | 301 |
| 276 | 79 | 96 | 210 | 212 | 212 | 305 |
| 277 | 90 | 88 | 210 | 201 | 201 | 301 |
| 278 | 83 | 128 | 212 | 201 | 201 | 303 |
| 279 | 118 | 41 | 202 | 202 | 212 | 302 |
| 280 | 83 | 5 | 205 | 201 | 201 | 302 |
| 281 | 10 | 99 | 203 | 202 | 212 | 307 |
| 282 | 118 | 119 | 212 | 212 | 212 | 302 |
| 283 | 129 | 71 | 211 | 202 | 212 | 306 |
| 284 | 79 | 65 | 212 | 212 | 212 | 306 |
| 285 | 130 | 16 | 205 | 201 | 201 | 306 |
| 286 | 83 | 27 | 212 | 202 | 212 | 306 |
| 287 | 129 | 51 | 211 | 201 | 201 | 307 |
| 288 | 90 | 26 | 209 | 212 | 212 | 303 |
| 289 | 83 | 84 | 203 | 212 | 212 | 303 |
| 290 | 83 | 101 | 203 | 201 | 201 | 301 |
| 291 | 118 | 81 | 210 | 212 | 212 | 301 |

Compounds having the formula:

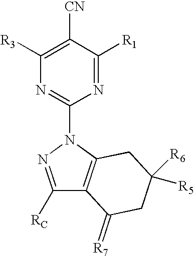

wherein R₁, R₃, R_{C}, R₄, and R₇ are defined in Table 2:

TABLE 2

| Compound | | | | | | |
|---|---|---|---|---|---|---|
| No. | R₁ | R₃ | R_{c} | R₅ | R₆ | R₇ |
| 292 | 129 | 101 | 212 | 202 | 212 | 307 |
| 293 | 10 | 118 | 203 | 212 | 212 | 306 |
| 294 | 118 | 43 | 212 | 202 | 212 | 308 |
| 295 | 90 | 26 | 209 | 212 | 212 | 303 |
| 296 | 10 | 98 | 212 | 201 | 201 | 307 |
| 297 | 118 | 130 | 206 | 202 | 212 | 306 |
| 298 | 83 | 30 | 210 | 201 | 201 | 305 |
| 299 | 83 | 5 | 205 | 201 | 201 | 302 |
| 300 | 83 | 36 | 212 | 201 | 201 | 301 |
| 301 | 130 | 56 | 209 | 202 | 212 | 304 |
| 302 | 118 | 47 | 203 | 202 | 212 | 306 |
| 303 | 118 | 114 | 212 | 202 | 212 | 304 |
| 304 | 129 | 91 | 201 | 201 | 201 | 305 |
| 305 | 118 | 119 | 212 | 212 | 212 | 302 |
| 306 | 129 | 58 | 202 | 212 | 212 | 306 |
| 307 | 10 | 98 | 205 | 201 | 201 | 302 |
| 308 | 79 | 61 | 202 | 202 | 212 | 301 |
| 309 | 90 | 82 | 209 | 202 | 212 | 302 |
| 310 | 118 | 37 | 204 | 202 | 212 | 306 |
| 311 | 130 | 106 | 205 | 212 | 212 | 307 |
| 312 | 129 | 51 | 211 | 201 | 201 | 307 |
| 313 | 10 | 32 | 211 | 212 | 212 | 301 |
| 314 | 10 | 109 | 207 | 201 | 201 | 306 |
| 315 | 130 | 69 | 210 | 201 | 201 | 303 |
| 316 | 130 | 29 | 210 | 202 | 212 | 306 |
| 317 | 129 | 72 | 204 | 202 | 212 | 305 |
| 318 | 83 | 84 | 203 | 212 | 212 | 303 |
| 319 | 90 | 53 | 204 | 201 | 201 | 303 |
| 320 | 90 | 39 | 204 | 212 | 212 | 302 |
| 321 | 118 | 19 | 202 | 201 | 201 | 308 |
| 322 | 10 | 80 | 204 | 212 | 212 | 306 |
| 323 | 79 | 126 | 212 | 202 | 212 | 307 |
| 324 | 83 | 98 | 209 | 201 | 201 | 307 |
| 325 | 90 | 100 | 212 | 212 | 212 | 308 |
| 326 | 90 | 23 | 205 | 202 | 212 | 301 |
| 327 | 129 | 20 | 201 | 201 | 201 | 305 |
| 328 | 83 | 88 | 209 | 212 | 212 | 307 |
| 329 | 10 | 109 | 201 | 212 | 212 | 301 |
| 330 | 79 | 96 | 210 | 212 | 212 | 305 |
| 331 | 90 | 86 | 210 | 212 | 212 | 303 |
| 332 | 130 | 89 | 206 | 202 | 212 | 306 |
| 333 | 130 | 117 | 211 | 202 | 212 | 302 |
| 334 | 79 | 79 | 206 | 212 | 212 | 301 |
| 335 | 118 | 76 | 202 | 202 | 212 | 306 |
| 336 | 90 | 21 | 212 | 202 | 212 | 301 |
| 337 | 90 | 96 | 212 | 212 | 212 | 307 |
| 338 | 118 | 105 | 212 | 202 | 212 | 306 |
| 339 | 79 | 97 | 210 | 201 | 201 | 304 |
| 340 | 118 | 108 | 205 | 212 | 212 | 302 |
| 341 | 118 | 81 | 210 | 212 | 212 | 301 |
| 342 | 10 | 77 | 204 | 212 | 212 | 301 |
| 343 | 90 | 122 | 201 | 201 | 201 | 308 |
| 344 | 90 | 85 | 204 | 202 | 212 | 301 |
| 345 | 129 | 85 | 204 | 212 | 212 | 301 |
| 346 | 130 | 101 | 205 | 202 | 212 | 302 |
| 347 | 83 | 88 | 206 | 212 | 212 | 301 |
| 348 | 129 | 9 | 212 | 212 | 212 | 305 |
| 349 | 129 | 55 | 204 | 201 | 201 | 302 |
| 350 | 83 | 44 | 202 | 201 | 201 | 307 |
| 351 | 10 | 48 | 212 | 202 | 212 | 303 |
| 352 | 10 | 86 | 212 | 212 | 212 | 301 |
| 353 | 10 | 91 | 209 | 212 | 212 | 302 |
| 354 | 130 | 85 | 210 | 202 | 212 | 303 |
| 355 | 83 | 128 | 212 | 201 | 201 | 303 |
| 356 | 90 | 12 | 207 | 201 | 201 | 303 |
| 357 | 79 | 88 | 206 | 202 | 212 | 303 |
| 358 | 118 | 102 | 201 | 201 | 201 | 308 |
| 359 | 130 | 112 | 212 | 201 | 201 | 304 |
| 360 | 10 | 52 | 201 | 202 | 212 | 306 |
| 361 | 90 | 94 | 201 | 212 | 212 | 304 |
| 362 | 90 | 96 | 204 | 201 | 201 | 308 |
| 363 | 118 | 73 | 210 | 202 | 212 | 308 |
| 364 | 90 | 101 | 207 | 212 | 212 | 301 |
| 365 | 83 | 91 | 206 | 212 | 212 | 307 |
| 366 | 130 | 88 | 211 | 201 | 201 | 306 |
| 367 | 118 | 41 | 202 | 202 | 212 | 302 |
| 368 | 118 | 67 | 211 | 201 | 201 | 302 |
| 369 | 83 | 113 | 210 | 201 | 201 | 307 |
| 370 | 118 | 120 | 201 | 201 | 201 | 304 |
| 371 | 90 | 101 | 201 | 212 | 212 | 308 |
| 372 | 129 | 62 | 205 | 212 | 212 | 303 |
| 373 | 83 | 17 | 207 | 201 | 201 | 308 |
| 374 | 90 | 2 | 201 | 201 | 201 | 308 |
| 375 | 10 | 11 | 212 | 212 | 212 | 306 |
| 376 | 90 | 86 | 204 | 212 | 212 | 304 |
| 377 | 118 | 87 | 206 | 202 | 212 | 302 |
| 378 | 90 | 13 | 204 | 201 | 201 | 302 |
| 379 | 83 | 33 | 203 | 202 | 212 | 302 |
| 380 | 118 | 85 | 211 | 212 | 212 | 301 |
| 381 | 83 | 4 | 201 | 201 | 201 | 304 |
| 382 | 10 | 1 | 201 | 212 | 212 | 308 |
| 383 | 90 | 91 | 210 | 201 | 201 | 308 |
| 384 | 130 | 104 | 210 | 202 | 212 | 307 |
| 385 | 129 | 109 | 207 | 201 | 201 | 304 |
| 386 | 10 | 99 | 203 | 202 | 212 | 307 |
| 387 | 10 | 91 | 203 | 201 | 201 | 306 |
| 388 | 90 | 98 | 204 | 212 | 212 | 301 |
| 389 | 79 | 127 | 210 | 201 | 201 | 301 |
| 390 | 79 | 24 | 204 | 201 | 201 | 301 |
| 391 | 83 | 54 | 212 | 201 | 201 | 302 |
| 392 | 90 | 66 | 201 | 212 | 212 | 304 |
| 393 | 90 | 78 | 204 | 201 | 201 | 307 |
| 394 | 130 | 25 | 204 | 202 | 212 | 306 |
| 395 | 79 | 65 | 212 | 212 | 212 | 306 |
| 396 | 129 | 88 | 208 | 212 | 212 | 301 |
| 397 | 83 | 8 | 204 | 212 | 212 | 301 |
| 398 | 79 | 96 | 212 | 201 | 201 | 301 |
| 399 | 90 | 101 | 211 | 201 | 201 | 306 |
| 400 | 79 | 86 | 201 | 202 | 212 | 301 |
| 401 | 129 | 28 | 212 | 202 | 212 | 302 |
| 402 | 10 | 22 | 211 | 201 | 201 | 308 |
| 403 | 118 | 35 | 209 | 202 | 212 | 301 |
| 404 | 10 | 74 | 210 | 201 | 201 | 303 |
| 405 | 129 | 71 | 211 | 202 | 212 | 306 |
| 406 | 83 | 109 | 207 | 201 | 201 | 302 |
| 407 | 129 | 6 | 210 | 202 | 212 | 306 |
| 408 | 118 | 18 | 202 | 202 | 212 | 306 |
| 409 | 83 | 129 | 211 | 202 | 212 | 302 |
| 410 | 90 | 7 | 204 | 201 | 201 | 307 |
| 411 | 118 | 42 | 205 | 212 | 212 | 308 |
| 412 | 10 | 125 | 201 | 212 | 212 | 308 |
| 413 | 118 | 88 | 210 | 202 | 212 | 306 |
| 414 | 83 | 15 | 211 | 212 | 212 | 302 |
| 415 | 90 | 70 | 205 | 201 | 201 | 302 |
| 416 | 90 | 83 | 211 | 212 | 212 | 303 |
| 417 | 129 | 40 | 210 | 202 | 212 | 306 |
| 418 | 130 | 123 | 201 | 201 | 201 | 307 |
| 419 | 79 | 109 | 204 | 201 | 201 | 303 |
| 420 | 90 | 111 | 204 | 202 | 212 | 308 |
| 421 | 118 | 64 | 212 | 201 | 201 | 308 |
| 422 | 130 | 50 | 212 | 202 | 212 | 307 |
| 423 | 90 | 90 | 205 | 212 | 212 | 305 |
| 424 | 130 | 16 | 205 | 201 | 201 | 306 |
| 425 | 90 | 103 | 205 | 201 | 201 | 301 |
| 426 | 90 | 10 | 211 | 202 | 212 | 308 |
| 427 | 90 | 96 | 211 | 201 | 201 | 301 |
| 428 | 83 | 86 | 201 | 212 | 212 | 302 |
| 429 | 79 | 91 | 212 | 201 | 201 | 304 |
| 430 | 130 | 14 | 203 | 201 | 201 | 308 |
| 431 | 90 | 86 | 205 | 201 | 201 | 303 |
| 432 | 83 | 38 | 206 | 201 | 201 | 305 |
| 433 | 130 | 115 | 206 | 201 | 201 | 308 |
| 434 | 83 | 27 | 212 | 202 | 212 | 306 |
| 435 | 90 | 96 | 201 | 201 | 201 | 304 |
| 436 | 129 | 107 | 204 | 201 | 201 | 302 |
| 437 | 118 | 57 | 202 | 212 | 212 | 308 |
| 438 | 10 | 45 | 205 | 201 | 201 | 303 |
| 439 | 79 | 116 | 205 | 202 | 212 | 301 |
| 440 | 129 | 92 | 204 | 212 | 212 | 301 |
| 441 | 83 | 101 | 203 | 201 | 201 | 301 |
| 442 | 10 | 46 | 204 | 202 | 212 | 308 |
| 443 | 10 | 85 | 212 | 212 | 212 | 307 |
| 444 | 118 | 49 | 209 | 201 | 201 | 307 |
| 445 | 118 | 59 | 206 | 201 | 201 | 306 |
| 446 | 118 | 34 | 205 | 201 | 201 | 304 |
| 447 | 129 | 68 | 204 | 212 | 212 | 304 |
| 448 | 10 | 96 | 209 | 201 | 201 | 302 |
| 449 | 130 | 121 | 209 | 202 | 212 | 308 |
| 450 | 130 | 31 | 208 | 212 | 212 | 305 |
| 451 | 83 | 98 | 208 | 201 | 201 | 301 |
| 452 | 10 | 3 | 203 | 202 | 212 | 302 |
| 453 | 10 | 85 | 204 | 212 | 212 | 308 |
| 454 | 118 | 96 | 201 | 201 | 201 | 308 |
| 455 | 118 | 109 | 204 | 212 | 212 | 303 |
| 456 | 118 | 63 | 210 | 202 | 212 | 308 |
| 457 | 130 | 109 | 212 | 201 | 201 | 305 |
| 458 | 83 | 60 | 202 | 201 | 201 | 301 |
| 459 | 130 | 93 | 205 | 212 | 212 | 307 |
| 460 | 130 | 88 | 204 | 202 | 212 | 306 |
| 461 | 90 | 88 | 210 | 201 | 201 | 301 |
| 462 | 83 | 124 | 203 | 201 | 201 | 308 |
| 463 | 79 | 110 | 207 | 201 | 201 | 302 |
| 464 | 79 | 75 | 204 | 202 | 212 | 302 |
| 465 | 10 | 98 | 208 | 201 | 201 | 308 |

Compounds having the formula:

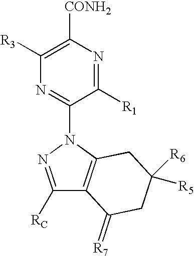

wherein R₁, R₃, R_{C}, R₆, and R₇ are defined in Table 3:

TABLE 3

| Compound | | | | | | |
|---|---|---|---|---|---|---|
| No. | R₁ | R₃ | R_{c} | R₅ | R₆ | R₇ |
| 466 | 130 | 89 | 206 | 202 | 212 | 306 |
| 467 | 90 | 82 | 209 | 202 | 212 | 302 |
| 468 | 118 | 34 | 205 | 201 | 201 | 304 |
| 469 | 90 | 88 | 210 | 201 | 201 | 301 |
| 470 | 83 | 88 | 209 | 212 | 212 | 307 |
| 471 | 10 | 3 | 203 | 202 | 212 | 302 |
| 472 | 90 | 26 | 209 | 212 | 212 | 303 |
| 473 | 90 | 122 | 201 | 201 | 201 | 308 |
| 474 | 10 | 109 | 207 | 201 | 201 | 306 |
| 475 | 118 | 57 | 202 | 212 | 212 | 308 |
| 476 | 83 | 27 | 212 | 202 | 212 | 306 |
| 477 | 10 | 98 | 208 | 201 | 201 | 308 |
| 478 | 130 | 88 | 204 | 202 | 212 | 306 |
| 479 | 130 | 69 | 210 | 201 | 201 | 303 |
| 480 | 90 | 96 | 211 | 201 | 201 | 301 |
| 481 | 90 | 10 | 211 | 202 | 212 | 308 |
| 482 | 90 | 13 | 204 | 201 | 201 | 302 |
| 483 | 118 | 130 | 206 | 202 | 212 | 306 |
| 484 | 130 | 112 | 212 | 201 | 201 | 304 |
| 485 | 10 | 86 | 212 | 212 | 212 | 301 |
| 486 | 79 | 116 | 205 | 202 | 212 | 301 |
| 487 | 118 | 73 | 210 | 202 | 212 | 308 |
| 488 | 130 | 106 | 205 | 212 | 212 | 307 |
| 489 | 10 | 48 | 212 | 202 | 212 | 303 |
| 490 | 79 | 86 | 201 | 202 | 212 | 301 |
| 491 | 83 | 44 | 202 | 201 | 201 | 307 |
| 492 | 90 | 100 | 212 | 212 | 212 | 308 |
| 493 | 118 | 43 | 212 | 202 | 212 | 308 |
| 494 | 90 | 101 | 207 | 212 | 212 | 301 |
| 495 | 90 | 86 | 210 | 212 | 212 | 303 |
| 496 | 90 | 23 | 205 | 202 | 212 | 301 |
| 497 | 90 | 96 | 204 | 201 | 201 | 308 |
| 498 | 118 | 42 | 205 | 212 | 212 | 308 |
| 499 | 79 | 96 | 212 | 201 | 201 | 301 |
| 500 | 118 | 96 | 201 | 201 | 201 | 308 |
| 501 | 130 | 14 | 203 | 201 | 201 | 308 |
| 502 | 83 | 128 | 212 | 201 | 201 | 303 |
| 503 | 79 | 126 | 212 | 202 | 212 | 307 |
| 504 | 90 | 96 | 201 | 201 | 201 | 304 |
| 505 | 79 | 79 | 206 | 212 | 212 | 301 |
| 506 | 118 | 18 | 202 | 202 | 212 | 306 |
| 507 | 118 | 64 | 212 | 201 | 201 | 308 |
| 508 | 118 | 19 | 202 | 201 | 201 | 308 |
| 509 | 129 | 68 | 204 | 212 | 212 | 304 |
| 510 | 83 | 30 | 210 | 201 | 201 | 305 |
| 511 | 118 | 119 | 212 | 212 | 212 | 302 |
| 512 | 10 | 99 | 203 | 202 | 212 | 307 |
| 513 | 90 | 53 | 204 | 201 | 201 | 303 |
| 514 | 129 | 85 | 204 | 212 | 212 | 301 |
| 515 | 10 | 32 | 211 | 212 | 212 | 301 |
| 516 | 90 | 101 | 201 | 212 | 212 | 308 |
| 517 | 90 | 70 | 205 | 201 | 201 | 302 |
| 518 | 79 | 24 | 204 | 201 | 201 | 301 |
| 519 | 130 | 93 | 205 | 212 | 212 | 307 |
| 520 | 83 | 33 | 203 | 202 | 212 | 302 |
| 521 | 90 | 86 | 204 | 212 | 212 | 304 |
| 522 | 83 | 113 | 210 | 201 | 201 | 307 |
| 523 | 130 | 88 | 211 | 201 | 201 | 306 |
| 524 | 118 | 67 | 211 | 201 | 201 | 302 |
| 525 | 130 | 85 | 210 | 202 | 212 | 303 |
| 526 | 118 | 35 | 209 | 202 | 212 | 301 |
| 527 | 129 | 62 | 205 | 212 | 212 | 303 |
| 528 | 129 | 20 | 201 | 201 | 201 | 305 |
| 529 | 118 | 114 | 212 | 202 | 212 | 304 |
| 530 | 10 | 85 | 212 | 212 | 212 | 307 |
| 531 | 83 | 86 | 201 | 212 | 212 | 302 |
| 532 | 83 | 38 | 206 | 201 | 201 | 305 |
| 533 | 79 | 110 | 207 | 201 | 201 | 302 |
| 534 | 83 | 88 | 206 | 212 | 212 | 301 |
| 535 | 129 | 71 | 211 | 202 | 212 | 306 |
| 536 | 129 | 51 | 211 | 201 | 201 | 307 |
| 537 | 130 | 50 | 212 | 202 | 212 | 307 |
| 538 | 129 | 72 | 204 | 202 | 212 | 305 |
| 539 | 83 | 124 | 203 | 201 | 201 | 308 |
| 540 | 90 | 91 | 210 | 201 | 201 | 308 |
| 541 | 10 | 80 | 204 | 212 | 212 | 306 |
| 542 | 83 | 129 | 211 | 202 | 212 | 302 |
| 543 | 10 | 91 | 203 | 201 | 201 | 306 |
| 544 | 83 | 8 | 204 | 212 | 212 | 301 |
| 545 | 10 | 118 | 203 | 212 | 212 | 306 |
| 546 | 118 | 49 | 209 | 201 | 201 | 307 |
| 547 | 130 | 16 | 205 | 201 | 201 | 306 |
| 548 | 83 | 109 | 207 | 201 | 201 | 302 |
| 549 | 10 | 1 | 201 | 212 | 212 | 308 |
| 550 | 118 | 102 | 201 | 201 | 201 | 308 |
| 551 | 118 | 105 | 212 | 202 | 212 | 306 |
| 552 | 79 | 75 | 204 | 202 | 212 | 302 |
| 553 | 90 | 111 | 204 | 202 | 212 | 308 |
| 554 | 10 | 74 | 210 | 201 | 201 | 303 |
| 555 | 118 | 87 | 206 | 202 | 212 | 302 |
| 556 | 129 | 58 | 202 | 212 | 212 | 306 |
| 557 | 129 | 9 | 212 | 212 | 212 | 305 |
| 558 | 118 | 63 | 210 | 202 | 212 | 308 |
| 559 | 129 | 6 | 210 | 202 | 212 | 306 |
| 560 | 79 | 88 | 206 | 202 | 212 | 303 |
| 561 | 79 | 91 | 212 | 201 | 201 | 304 |
| 562 | 90 | 85 | 204 | 202 | 212 | 301 |
| 563 | 129 | 109 | 207 | 201 | 201 | 304 |
| 564 | 10 | 109 | 201 | 212 | 212 | 301 |
| 565 | 129 | 107 | 204 | 201 | 201 | 302 |
| 566 | 90 | 78 | 204 | 201 | 201 | 307 |
| 567 | 129 | 88 | 208 | 212 | 212 | 301 |
| 568 | 10 | 98 | 212 | 201 | 201 | 307 |
| 569 | 10 | 98 | 205 | 201 | 201 | 302 |
| 570 | 118 | 41 | 202 | 202 | 212 | 302 |
| 571 | 83 | 4 | 201 | 201 | 201 | 304 |
| 572 | 90 | 103 | 205 | 201 | 201 | 301 |
| 573 | 90 | 90 | 205 | 212 | 212 | 305 |
| 574 | 118 | 81 | 210 | 212 | 212 | 301 |
| 575 | 10 | 85 | 204 | 212 | 212 | 308 |
| 576 | 79 | 127 | 210 | 201 | 201 | 301 |
| 577 | 129 | 91 | 201 | 201 | 201 | 305 |
| 578 | 130 | 56 | 209 | 202 | 212 | 304 |
| 579 | 118 | 85 | 211 | 212 | 212 | 301 |
| 580 | 90 | 98 | 204 | 212 | 212 | 301 |
| 581 | 90 | 7 | 204 | 201 | 201 | 307 |
| 582 | 83 | 91 | 206 | 212 | 212 | 307 |
| 583 | 118 | 108 | 205 | 212 | 212 | 302 |
| 584 | 129 | 40 | 210 | 202 | 212 | 306 |
| 585 | 129 | 92 | 204 | 212 | 212 | 301 |
| 586 | 83 | 15 | 211 | 212 | 212 | 302 |
| 587 | 83 | 101 | 203 | 201 | 201 | 301 |
| 588 | 83 | 36 | 212 | 201 | 201 | 301 |
| 589 | 10 | 96 | 209 | 201 | 201 | 302 |
| 590 | 118 | 59 | 206 | 201 | 201 | 306 |
| 591 | 83 | 84 | 203 | 212 | 212 | 303 |
| 592 | 83 | 17 | 207 | 201 | 201 | 308 |
| 593 | 118 | 120 | 201 | 201 | 201 | 304 |
| 594 | 90 | 83 | 211 | 212 | 212 | 303 |
| 595 | 130 | 115 | 206 | 201 | 201 | 308 |
| 596 | 10 | 125 | 201 | 212 | 212 | 308 |
| 597 | 130 | 29 | 210 | 202 | 212 | 306 |
| 598 | 90 | 21 | 212 | 202 | 212 | 301 |
| 599 | 130 | 31 | 208 | 212 | 212 | 305 |
| 600 | 118 | 76 | 202 | 202 | 212 | 306 |
| 601 | 90 | 94 | 201 | 212 | 212 | 304 |
| 602 | 130 | 25 | 204 | 202 | 212 | 306 |
| 603 | 83 | 98 | 209 | 201 | 201 | 307 |
| 604 | 90 | 66 | 201 | 212 | 212 | 304 |
| 605 | 90 | 2 | 201 | 201 | 201 | 308 |
| 606 | 83 | 54 | 212 | 201 | 201 | 302 |
| 607 | 83 | 98 | 208 | 201 | 201 | 301 |
| 608 | 79 | 65 | 212 | 212 | 212 | 306 |
| 609 | 10 | 46 | 204 | 202 | 212 | 308 |
| 610 | 129 | 28 | 212 | 202 | 212 | 302 |
| 611 | 90 | 39 | 204 | 212 | 212 | 302 |
| 612 | 83 | 5 | 205 | 201 | 201 | 302 |
| 613 | 130 | 104 | 210 | 202 | 212 | 307 |
| 614 | 90 | 101 | 211 | 201 | 201 | 306 |
| 615 | 79 | 97 | 210 | 201 | 201 | 304 |
| 616 | 79 | 109 | 204 | 201 | 201 | 303 |
| 617 | 79 | 96 | 210 | 212 | 212 | 305 |
| 618 | 90 | 86 | 205 | 201 | 201 | 303 |
| 619 | 130 | 109 | 212 | 201 | 201 | 305 |
| 620 | 130 | 95 | 204 | 201 | 201 | 308 |
| 621 | 130 | 123 | 201 | 201 | 201 | 307 |
| 622 | 10 | 45 | 205 | 201 | 201 | 303 |
| 623 | 118 | 37 | 204 | 202 | 212 | 306 |
| 624 | 118 | 109 | 204 | 212 | 212 | 303 |
| 625 | 129 | 101 | 212 | 202 | 212 | 307 |
| 626 | 130 | 121 | 209 | 202 | 212 | 308 |
| 627 | 118 | 47 | 203 | 202 | 212 | 306 |
| 628 | 130 | 101 | 205 | 202 | 212 | 302 |
| 629 | 10 | 52 | 201 | 202 | 212 | 306 |
| 630 | 79 | 61 | 202 | 202 | 212 | 301 |
| 631 | 10 | 91 | 209 | 212 | 212 | 302 |
| 632 | 90 | 12 | 207 | 201 | 201 | 303 |
| 633 | 118 | 88 | 210 | 202 | 212 | 306 |
| 634 | 130 | 117 | 211 | 202 | 212 | 302 |
| 635 | 10 | 11 | 212 | 212 | 212 | 306 |
| 636 | 10 | 77 | 204 | 212 | 212 | 301 |
| 637 | 90 | 96 | 212 | 212 | 212 | 307 |
| 638 | 83 | 60 | 202 | 201 | 201 | 301 |
| 639 | 129 | 55 | 204 | 201 | 201 | 302 |
| 640 | 10 | 22 | 211 | 201 | 201 | 308 |

Compounds having the formula:

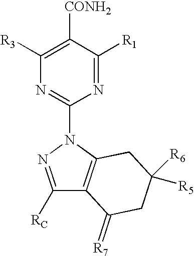

wherein R₁, R₃, R_{C}, R₅, R₆, and R₇ are defined in Table 4:

TABLE 4

| Compound | | | | | | |
|---|---|---|---|---|---|---|
| No. | R₁ | R₃ | R_{c} | R₅ | R₆ | R₇ |
| 641 | 118 | 96 | 201 | 201 | 201 | 308 |
| 642 | 83 | 124 | 203 | 201 | 201 | 308 |
| 643 | 83 | 36 | 212 | 201 | 201 | 301 |
| 644 | 130 | 121 | 209 | 202 | 212 | 308 |
| 645 | 130 | 56 | 209 | 202 | 212 | 304 |
| 646 | 83 | 38 | 206 | 201 | 201 | 305 |
| 647 | 90 | 83 | 211 | 212 | 212 | 303 |
| 648 | 118 | 49 | 209 | 201 | 201 | 307 |
| 649 | 79 | 86 | 201 | 202 | 212 | 301 |
| 650 | 130 | 104 | 210 | 202 | 212 | 307 |
| 651 | 130 | 50 | 212 | 202 | 212 | 307 |
| 652 | 118 | 42 | 205 | 212 | 212 | 308 |
| 653 | 79 | 97 | 210 | 201 | 201 | 304 |
| 654 | 118 | 114 | 212 | 202 | 212 | 304 |
| 655 | 79 | 88 | 206 | 202 | 212 | 303 |
| 656 | 90 | 82 | 209 | 202 | 212 | 302 |
| 657 | 129 | 85 | 204 | 212 | 212 | 301 |
| 658 | 118 | 130 | 206 | 202 | 212 | 306 |
| 659 | 130 | 115 | 206 | 201 | 201 | 308 |
| 660 | 83 | 54 | 212 | 201 | 201 | 302 |
| 661 | 10 | 98 | 205 | 201 | 201 | 302 |
| 662 | 90 | 101 | 211 | 201 | 201 | 306 |
| 663 | 83 | 5 | 205 | 201 | 201 | 302 |
| 664 | 10 | 1 | 201 | 212 | 212 | 308 |
| 665 | 118 | 57 | 202 | 212 | 212 | 308 |
| 666 | 83 | 129 | 211 | 202 | 212 | 302 |
| 667 | 79 | 61 | 202 | 202 | 212 | 301 |
| 668 | 129 | 9 | 212 | 212 | 212 | 305 |
| 669 | 130 | 101 | 205 | 202 | 212 | 302 |
| 670 | 90 | 66 | 201 | 212 | 212 | 304 |
| 671 | 118 | 88 | 210 | 202 | 212 | 306 |
| 672 | 130 | 88 | 211 | 201 | 201 | 306 |
| 673 | 83 | 60 | 202 | 201 | 201 | 301 |
| 674 | 90 | 53 | 204 | 201 | 201 | 303 |
| 675 | 10 | 98 | 208 | 201 | 201 | 308 |
| 676 | 130 | 85 | 210 | 202 | 212 | 303 |
| 677 | 118 | 19 | 202 | 201 | 201 | 308 |
| 678 | 130 | 89 | 206 | 202 | 212 | 306 |
| 679 | 90 | 96 | 211 | 201 | 201 | 301 |
| 680 | 10 | 86 | 212 | 212 | 212 | 301 |
| 681 | 83 | 113 | 210 | 201 | 201 | 307 |
| 682 | 90 | 39 | 204 | 212 | 212 | 302 |
| 683 | 118 | 73 | 210 | 202 | 212 | 308 |
| 684 | 10 | 99 | 203 | 202 | 212 | 307 |
| 685 | 10 | 22 | 211 | 201 | 201 | 308 |
| 686 | 118 | 105 | 212 | 202 | 212 | 306 |
| 687 | 83 | 84 | 203 | 212 | 212 | 303 |
| 688 | 10 | 74 | 210 | 201 | 201 | 303 |
| 689 | 83 | 27 | 212 | 202 | 212 | 306 |
| 690 | 129 | 92 | 204 | 212 | 212 | 301 |
| 691 | 10 | 3 | 203 | 202 | 212 | 302 |
| 692 | 10 | 85 | 212 | 212 | 212 | 307 |
| 693 | 79 | 75 | 204 | 202 | 212 | 302 |
| 694 | 10 | 109 | 201 | 212 | 212 | 301 |
| 695 | 10 | 98 | 212 | 201 | 201 | 307 |
| 696 | 90 | 86 | 210 | 212 | 212 | 303 |
| 697 | 90 | 94 | 201 | 212 | 212 | 304 |
| 698 | 129 | 62 | 205 | 212 | 212 | 303 |
| 699 | 130 | 69 | 210 | 201 | 201 | 303 |
| 700 | 83 | 128 | 212 | 201 | 201 | 303 |
| 701 | 118 | 63 | 210 | 202 | 212 | 308 |
| 702 | 129 | 91 | 201 | 201 | 201 | 305 |
| 703 | 129 | 51 | 211 | 201 | 201 | 307 |
| 704 | 10 | 91 | 209 | 212 | 212 | 302 |
| 705 | 118 | 109 | 204 | 212 | 212 | 303 |
| 706 | 129 | 6 | 210 | 202 | 212 | 306 |
| 707 | 10 | 118 | 203 | 212 | 212 | 306 |
| 708 | 90 | 13 | 204 | 201 | 201 | 302 |
| 709 | 129 | 68 | 204 | 212 | 212 | 304 |
| 710 | 130 | 123 | 201 | 201 | 201 | 307 |
| 711 | 118 | 81 | 210 | 212 | 212 | 301 |
| 712 | 90 | 86 | 205 | 201 | 201 | 303 |
| 713 | 83 | 88 | 206 | 212 | 212 | 301 |
| 714 | 130 | 106 | 205 | 212 | 212 | 307 |
| 715 | 118 | 64 | 212 | 201 | 201 | 308 |
| 716 | 83 | 30 | 210 | 201 | 201 | 305 |
| 717 | 10 | 77 | 204 | 212 | 212 | 301 |
| 718 | 90 | 21 | 212 | 202 | 212 | 301 |
| 719 | 90 | 122 | 201 | 201 | 201 | 308 |
| 720 | 118 | 37 | 204 | 202 | 212 | 306 |
| 721 | 129 | 28 | 212 | 202 | 212 | 302 |
| 722 | 118 | 85 | 211 | 212 | 212 | 301 |
| 723 | 10 | 32 | 211 | 212 | 212 | 301 |
| 724 | 129 | 40 | 210 | 202 | 212 | 306 |
| 725 | 10 | 125 | 201 | 212 | 212 | 308 |
| 726 | 129 | 101 | 212 | 202 | 212 | 307 |
| 727 | 90 | 90 | 205 | 212 | 212 | 305 |
| 728 | 130 | 88 | 204 | 202 | 212 | 306 |
| 729 | 90 | 78 | 204 | 201 | 201 | 307 |
| 730 | 118 | 18 | 202 | 202 | 212 | 306 |
| 731 | 130 | 25 | 204 | 202 | 212 | 306 |
| 732 | 79 | 65 | 212 | 212 | 212 | 306 |
| 733 | 90 | 2 | 201 | 201 | 201 | 308 |
| 734 | 90 | 101 | 201 | 212 | 212 | 308 |
| 735 | 83 | 8 | 204 | 212 | 212 | 301 |
| 736 | 90 | 10 | 211 | 202 | 212 | 308 |
| 737 | 118 | 76 | 202 | 202 | 212 | 306 |
| 738 | 10 | 85 | 204 | 212 | 212 | 308 |
| 739 | 129 | 55 | 204 | 201 | 201 | 302 |
| 740 | 90 | 88 | 210 | 201 | 201 | 301 |
| 741 | 129 | 72 | 204 | 202 | 212 | 305 |
| 742 | 83 | 17 | 207 | 201 | 201 | 308 |
| 743 | 118 | 47 | 203 | 202 | 212 | 306 |
| 744 | 79 | 127 | 210 | 201 | 201 | 301 |
| 745 | 10 | 48 | 212 | 202 | 212 | 303 |
| 746 | 90 | 100 | 212 | 212 | 212 | 308 |
| 747 | 10 | 109 | 207 | 201 | 201 | 306 |
| 748 | 129 | 88 | 208 | 212 | 212 | 301 |
| 749 | 83 | 98 | 209 | 201 | 201 | 307 |
| 750 | 129 | 20 | 201 | 201 | 201 | 305 |
| 751 | 118 | 41 | 202 | 202 | 212 | 302 |
| 752 | 90 | 111 | 204 | 202 | 212 | 308 |
| 753 | 118 | 59 | 206 | 201 | 201 | 306 |
| 754 | 90 | 96 | 212 | 212 | 212 | 307 |
| 755 | 118 | 43 | 212 | 202 | 212 | 308 |
| 756 | 79 | 96 | 210 | 212 | 212 | 305 |
| 757 | 10 | 96 | 209 | 201 | 201 | 302 |
| 758 | 79 | 126 | 212 | 202 | 212 | 307 |
| 759 | 83 | 33 | 203 | 202 | 212 | 302 |
| 760 | 90 | 70 | 205 | 201 | 201 | 302 |
| 761 | 118 | 108 | 205 | 212 | 212 | 302 |
| 762 | 129 | 58 | 202 | 212 | 212 | 306 |
| 763 | 10 | 45 | 205 | 201 | 201 | 303 |
| 764 | 90 | 23 | 205 | 202 | 212 | 301 |
| 765 | 10 | 46 | 204 | 202 | 212 | 308 |
| 766 | 83 | 4 | 201 | 201 | 201 | 304 |
| 767 | 90 | 91 | 210 | 201 | 201 | 308 |
| 768 | 10 | 11 | 212 | 212 | 212 | 306 |
| 769 | 118 | 87 | 206 | 202 | 212 | 302 |
| 770 | 129 | 71 | 211 | 202 | 212 | 306 |
| 771 | 118 | 35 | 209 | 202 | 212 | 301 |
| 772 | 90 | 12 | 207 | 201 | 201 | 303 |
| 773 | 118 | 34 | 205 | 201 | 201 | 304 |
| 774 | 79 | 116 | 205 | 202 | 212 | 301 |
| 775 | 90 | 26 | 209 | 212 | 212 | 303 |
| 776 | 79 | 110 | 207 | 201 | 201 | 302 |
| 777 | 118 | 67 | 211 | 201 | 201 | 302 |
| 778 | 83 | 98 | 208 | 201 | 201 | 301 |
| 779 | 130 | 16 | 205 | 201 | 201 | 306 |
| 780 | 130 | 117 | 211 | 202 | 212 | 302 |
| 781 | 83 | 88 | 209 | 212 | 212 | 307 |
| 782 | 83 | 44 | 202 | 201 | 201 | 307 |
| 783 | 10 | 80 | 204 | 212 | 212 | 306 |
| 784 | 90 | 85 | 204 | 202 | 212 | 301 |
| 785 | 90 | 101 | 207 | 212 | 212 | 301 |
| 786 | 130 | 109 | 212 | 201 | 201 | 305 |
| 787 | 83 | 91 | 206 | 212 | 212 | 307 |
| 788 | 130 | 95 | 204 | 201 | 201 | 308 |
| 789 | 90 | 96 | 204 | 201 | 201 | 308 |
| 790 | 129 | 109 | 207 | 201 | 201 | 304 |
| 791 | 90 | 103 | 205 | 201 | 201 | 301 |
| 792 | 90 | 98 | 204 | 212 | 212 | 301 |
| 793 | 79 | 109 | 204 | 201 | 201 | 303 |
| 794 | 83 | 15 | 211 | 212 | 212 | 302 |
| 795 | 83 | 101 | 203 | 201 | 201 | 301 |
| 796 | 10 | 52 | 201 | 202 | 212 | 306 |
| 797 | 90 | 96 | 201 | 201 | 201 | 304 |
| 798 | 90 | 7 | 204 | 201 | 201 | 307 |
| 799 | 10 | 91 | 203 | 201 | 201 | 306 |
| 800 | 130 | 93 | 205 | 212 | 212 | 307 |
| 801 | 79 | 24 | 204 | 201 | 201 | 301 |
| 802 | 129 | 107 | 204 | 201 | 201 | 302 |
| 803 | 118 | 102 | 201 | 201 | 201 | 308 |
| 804 | 90 | 86 | 204 | 212 | 212 | 304 |
| 805 | 83 | 86 | 201 | 212 | 212 | 302 |
| 806 | 130 | 29 | 210 | 202 | 212 | 306 |
| 807 | 83 | 109 | 207 | 201 | 201 | 302 |
| 808 | 79 | 79 | 206 | 212 | 212 | 301 |
| 809 | 79 | 91 | 212 | 201 | 201 | 304 |
| 810 | 118 | 119 | 212 | 212 | 212 | 302 |
| 811 | 79 | 96 | 212 | 201 | 201 | 301 |
| 812 | 130 | 14 | 203 | 201 | 201 | 308 |
| 813 | 118 | 120 | 201 | 201 | 201 | 304 |
| 814 | 130 | 112 | 212 | 201 | 201 | 304 |
| 815 | 130 | 31 | 208 | 212 | 212 | 305 |

Compounds having the formula:

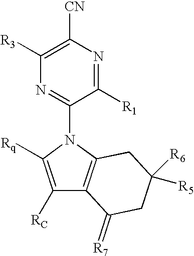

wherein R₁, R₃, R_{C}, R₅, R₆, and R₇ are defined in Table 5:

TABLE 5

| Compound | | | | | | | |
|---|---|---|---|---|---|---|---|
| No. | R₁ | R₃ | R_{c} | R_{q} | R₅ | R₆ | R₇ |
| 816 | 118 | 37 | 204 | 202 | 202 | 212 | 306 |
| 817 | 79 | 61 | 202 | 212 | 202 | 212 | 301 |
| 818 | 90 | 96 | 211 | 201 | 201 | 201 | 301 |
| 819 | 118 | 120 | 201 | 201 | 201 | 201 | 304 |
| 820 | 79 | 109 | 204 | 209 | 201 | 201 | 303 |
| 821 | 118 | 47 | 203 | 202 | 202 | 212 | 306 |
| 822 | 118 | 64 | 212 | 202 | 201 | 201 | 308 |
| 823 | 10 | 32 | 211 | 201 | 212 | 212 | 301 |
| 824 | 90 | 66 | 201 | 212 | 212 | 212 | 304 |
| 825 | 79 | 96 | 212 | 201 | 201 | 201 | 301 |
| 826 | 118 | 41 | 202 | 209 | 202 | 212 | 302 |
| 827 | 130 | 101 | 205 | 201 | 202 | 212 | 302 |
| 828 | 130 | 16 | 205 | 212 | 201 | 201 | 306 |
| 829 | 79 | 97 | 210 | 202 | 201 | 201 | 304 |
| 830 | 90 | 10 | 211 | 201 | 202 | 212 | 308 |
| 831 | 118 | 119 | 212 | 201 | 212 | 212 | 302 |
| 832 | 118 | 114 | 212 | 212 | 202 | 212 | 304 |
| 833 | 83 | 124 | 203 | 201 | 201 | 201 | 308 |
| 834 | 130 | 88 | 204 | 201 | 202 | 212 | 306 |
| 835 | 79 | 75 | 204 | 209 | 202 | 212 | 302 |
| 836 | 130 | 115 | 206 | 202 | 201 | 201 | 308 |
| 837 | 90 | 86 | 210 | 202 | 212 | 212 | 303 |
| 838 | 129 | 101 | 212 | 209 | 202 | 212 | 307 |
| 839 | 130 | 95 | 204 | 212 | 201 | 201 | 308 |
| 840 | 10 | 80 | 204 | 212 | 212 | 212 | 306 |
| 841 | 83 | 101 | 203 | 201 | 201 | 201 | 301 |
| 842 | 118 | 87 | 206 | 212 | 202 | 212 | 302 |
| 843 | 129 | 6 | 210 | 201 | 202 | 212 | 306 |
| 844 | 90 | 101 | 207 | 212 | 212 | 212 | 301 |
| 845 | 90 | 101 | 211 | 202 | 201 | 201 | 306 |
| 846 | 83 | 88 | 209 | 202 | 212 | 212 | 307 |
| 847 | 83 | 15 | 211 | 212 | 212 | 212 | 302 |
| 848 | 90 | 88 | 210 | 212 | 201 | 201 | 301 |
| 849 | 10 | 11 | 212 | 201 | 212 | 212 | 306 |
| 850 | 129 | 40 | 210 | 209 | 202 | 212 | 306 |
| 851 | 129 | 72 | 204 | 202 | 202 | 212 | 305 |
| 852 | 118 | 59 | 206 | 202 | 201 | 201 | 306 |
| 853 | 130 | 121 | 209 | 202 | 202 | 212 | 308 |
| 854 | 90 | 103 | 205 | 212 | 201 | 201 | 301 |
| 855 | 130 | 31 | 208 | 202 | 212 | 212 | 305 |
| 856 | 83 | 17 | 207 | 202 | 201 | 201 | 308 |
| 857 | 118 | 105 | 212 | 201 | 202 | 212 | 306 |
| 858 | 129 | 51 | 211 | 212 | 201 | 201 | 307 |
| 859 | 90 | 12 | 207 | 201 | 201 | 201 | 303 |
| 860 | 118 | 96 | 201 | 201 | 201 | 201 | 308 |
| 861 | 90 | 53 | 204 | 201 | 201 | 201 | 303 |
| 862 | 90 | 96 | 201 | 212 | 201 | 201 | 304 |
| 863 | 83 | 98 | 208 | 212 | 201 | 201 | 301 |
| 864 | 90 | 86 | 205 | 202 | 201 | 201 | 303 |
| 865 | 10 | 109 | 201 | 202 | 212 | 212 | 301 |
| 866 | 83 | 36 | 212 | 209 | 201 | 201 | 301 |
| 867 | 90 | 70 | 205 | 201 | 201 | 201 | 302 |
| 868 | 79 | 86 | 201 | 212 | 202 | 212 | 301 |
| 869 | 83 | 84 | 203 | 201 | 212 | 212 | 303 |
| 870 | 79 | 88 | 206 | 201 | 202 | 212 | 303 |
| 871 | 129 | 58 | 202 | 202 | 212 | 212 | 306 |
| 872 | 130 | 112 | 212 | 212 | 201 | 201 | 304 |
| 873 | 83 | 128 | 212 | 209 | 201 | 201 | 303 |
| 874 | 79 | 79 | 206 | 202 | 212 | 212 | 301 |
| 875 | 10 | 125 | 201 | 212 | 212 | 212 | 308 |
| 876 | 83 | 88 | 206 | 209 | 212 | 212 | 301 |
| 877 | 90 | 85 | 204 | 202 | 202 | 212 | 301 |
| 878 | 10 | 85 | 212 | 212 | 212 | 212 | 307 |
| 879 | 129 | 55 | 204 | 212 | 201 | 201 | 302 |
| 880 | 10 | 91 | 209 | 209 | 212 | 212 | 302 |
| 881 | 83 | 38 | 206 | 201 | 201 | 201 | 305 |
| 882 | 79 | 91 | 212 | 201 | 201 | 201 | 304 |
| 883 | 118 | 34 | 205 | 212 | 201 | 201 | 304 |
| 884 | 118 | 109 | 204 | 202 | 212 | 212 | 303 |
| 885 | 118 | 43 | 212 | 212 | 202 | 212 | 308 |
| 886 | 118 | 102 | 201 | 209 | 201 | 201 | 308 |
| 887 | 10 | 109 | 207 | 201 | 201 | 201 | 306 |
| 888 | 10 | 85 | 204 | 201 | 212 | 212 | 308 |
| 889 | 129 | 88 | 208 | 212 | 212 | 212 | 301 |
| 890 | 90 | 122 | 201 | 212 | 201 | 201 | 308 |
| 891 | 129 | 20 | 201 | 209 | 201 | 201 | 305 |
| 892 | 10 | 99 | 203 | 201 | 202 | 212 | 307 |
| 893 | 10 | 98 | 212 | 212 | 201 | 201 | 307 |
| 894 | 79 | 65 | 212 | 201 | 212 | 212 | 306 |
| 895 | 118 | 19 | 202 | 212 | 201 | 201 | 308 |
| 896 | 79 | 127 | 210 | 202 | 201 | 201 | 301 |
| 897 | 90 | 86 | 204 | 202 | 212 | 212 | 304 |
| 898 | 118 | 108 | 205 | 209 | 212 | 212 | 302 |
| 899 | 90 | 7 | 204 | 201 | 201 | 201 | 307 |
| 900 | 130 | 106 | 205 | 209 | 212 | 212 | 307 |
| 901 | 129 | 109 | 207 | 209 | 201 | 201 | 304 |
| 902 | 90 | 2 | 201 | 212 | 201 | 201 | 308 |
| 903 | 129 | 107 | 204 | 201 | 201 | 201 | 302 |
| 904 | 10 | 3 | 203 | 201 | 202 | 212 | 302 |
| 905 | 118 | 35 | 209 | 201 | 202 | 212 | 301 |
| 906 | 83 | 44 | 202 | 201 | 201 | 201 | 307 |
| 907 | 118 | 73 | 210 | 209 | 202 | 212 | 308 |
| 908 | 90 | 78 | 204 | 201 | 201 | 201 | 307 |
| 909 | 90 | 98 | 204 | 202 | 212 | 212 | 301 |
| 910 | 129 | 85 | 204 | 201 | 212 | 212 | 301 |
| 911 | 130 | 69 | 210 | 202 | 201 | 201 | 303 |
| 912 | 83 | 60 | 202 | 209 | 201 | 201 | 301 |
| 913 | 129 | 71 | 211 | 202 | 202 | 212 | 306 |
| 914 | 79 | 116 | 205 | 212 | 202 | 212 | 301 |
| 915 | 118 | 42 | 205 | 201 | 212 | 212 | 308 |
| 916 | 10 | 86 | 212 | 201 | 212 | 212 | 301 |
| 917 | 130 | 89 | 206 | 202 | 202 | 212 | 306 |
| 918 | 83 | 129 | 211 | 202 | 202 | 212 | 302 |
| 919 | 10 | 74 | 210 | 209 | 201 | 201 | 303 |
| 920 | 83 | 4 | 201 | 212 | 201 | 201 | 304 |
| 921 | 129 | 91 | 201 | 212 | 201 | 201 | 305 |
| 922 | 90 | 96 | 204 | 202 | 201 | 201 | 308 |
| 923 | 90 | 21 | 212 | 212 | 202 | 212 | 301 |
| 924 | 90 | 13 | 204 | 212 | 201 | 201 | 302 |
| 925 | 130 | 56 | 209 | 202 | 202 | 212 | 304 |
| 926 | 118 | 18 | 202 | 201 | 202 | 212 | 306 |
| 927 | 90 | 91 | 210 | 212 | 201 | 201 | 308 |
| 928 | 129 | 92 | 204 | 202 | 212 | 212 | 301 |
| 929 | 90 | 100 | 212 | 209 | 212 | 212 | 308 |
| 930 | 79 | 96 | 210 | 202 | 212 | 212 | 305 |
| 931 | 118 | 85 | 211 | 202 | 212 | 212 | 301 |
| 932 | 10 | 1 | 201 | 212 | 212 | 212 | 308 |
| 933 | 90 | 101 | 201 | 201 | 212 | 212 | 308 |
| 934 | 130 | 85 | 210 | 212 | 202 | 212 | 303 |
| 935 | 118 | 63 | 210 | 212 | 202 | 212 | 308 |
| 936 | 118 | 57 | 202 | 202 | 212 | 212 | 308 |
| 937 | 90 | 83 | 211 | 202 | 212 | 212 | 303 |
| 938 | 83 | 86 | 201 | 212 | 212 | 212 | 302 |
| 939 | 90 | 94 | 201 | 201 | 212 | 212 | 304 |
| 940 | 79 | 110 | 207 | 201 | 201 | 201 | 302 |
| 941 | 83 | 91 | 206 | 209 | 212 | 212 | 307 |
| 942 | 130 | 109 | 212 | 201 | 201 | 201 | 305 |
| 943 | 118 | 67 | 211 | 202 | 201 | 201 | 302 |
| 944 | 83 | 109 | 207 | 209 | 201 | 201 | 302 |
| 945 | 130 | 93 | 205 | 202 | 212 | 212 | 307 |
| 946 | 130 | 88 | 211 | 202 | 201 | 201 | 306 |
| 947 | 90 | 39 | 204 | 201 | 212 | 212 | 302 |
| 948 | 10 | 91 | 203 | 201 | 201 | 201 | 306 |
| 949 | 79 | 24 | 204 | 209 | 201 | 201 | 301 |
| 950 | 83 | 5 | 205 | 201 | 201 | 201 | 302 |
| 951 | 10 | 22 | 211 | 202 | 201 | 201 | 308 |
| 952 | 129 | 68 | 204 | 201 | 212 | 212 | 304 |
| 953 | 90 | 26 | 209 | 202 | 212 | 212 | 303 |
| 954 | 130 | 29 | 210 | 201 | 202 | 212 | 306 |
| 955 | 90 | 111 | 204 | 212 | 202 | 212 | 308 |
| 956 | 10 | 96 | 209 | 201 | 201 | 201 | 302 |
| 957 | 10 | 45 | 205 | 202 | 201 | 201 | 303 |
| 958 | 90 | 82 | 209 | 201 | 202 | 212 | 302 |
| 959 | 130 | 25 | 204 | 202 | 202 | 212 | 306 |
| 960 | 10 | 77 | 204 | 202 | 212 | 212 | 301 |
| 961 | 118 | 81 | 210 | 212 | 212 | 212 | 301 |
| 962 | 83 | 98 | 209 | 212 | 201 | 201 | 307 |
| 963 | 10 | 98 | 205 | 212 | 201 | 201 | 302 |
| 964 | 129 | 28 | 212 | 212 | 202 | 212 | 302 |
| 965 | 130 | 14 | 203 | 201 | 201 | 201 | 308 |
| 966 | 130 | 50 | 212 | 209 | 202 | 212 | 307 |
| 967 | 83 | 30 | 210 | 212 | 201 | 201 | 305 |
| 968 | 90 | 96 | 212 | 209 | 212 | 212 | 307 |
| 969 | 83 | 33 | 203 | 209 | 202 | 212 | 302 |
| 970 | 118 | 49 | 209 | 201 | 201 | 201 | 307 |
| 971 | 90 | 90 | 205 | 209 | 212 | 212 | 305 |
| 972 | 83 | 8 | 204 | 209 | 212 | 212 | 301 |
| 973 | 90 | 23 | 205 | 209 | 202 | 212 | 301 |
| 974 | 129 | 9 | 212 | 202 | 212 | 212 | 305 |
| 975 | 118 | 88 | 210 | 202 | 202 | 212 | 306 |
| 976 | 79 | 126 | 212 | 209 | 202 | 212 | 307 |
| 977 | 118 | 130 | 206 | 202 | 202 | 212 | 306 |
| 978 | 130 | 117 | 211 | 212 | 202 | 212 | 302 |
| 979 | 10 | 48 | 212 | 201 | 202 | 212 | 303 |
| 980 | 130 | 104 | 210 | 202 | 202 | 212 | 307 |
| 981 | 10 | 118 | 203 | 202 | 212 | 212 | 306 |
| 982 | 130 | 123 | 201 | 202 | 201 | 201 | 307 |
| 983 | 83 | 54 | 212 | 212 | 201 | 201 | 302 |
| 984 | 83 | 27 | 212 | 212 | 202 | 212 | 306 |
| 985 | 10 | 52 | 201 | 202 | 202 | 212 | 306 |
| 986 | 129 | 62 | 205 | 212 | 212 | 212 | 303 |
| 987 | 83 | 113 | 210 | 202 | 201 | 201 | 307 |
| 988 | 118 | 76 | 202 | 209 | 202 | 212 | 306 |
| 989 | 10 | 98 | 208 | 212 | 201 | 201 | 308 |
| 990 | 10 | 46 | 204 | 209 | 202 | 212 | 308 |

Compounds having the formula:

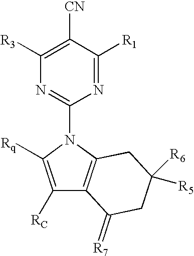

wherein R₁, R₃, R_{C}, R₅, R₆, and R₇ are defined in Table 6:

TABLE 6

| Compound No. | R₁ | R₃ | R_{c} | R_{q} | R₅ | R₆ | R₇ |
|---|---|---|---|---|---|---|---|
| 991 | 90 | 70 | 205 | 201 | 201 | 201 | 302 |
| 992 | 79 | 65 | 212 | 201 | 212 | 212 | 306 |
| 993 | 90 | 96 | 212 | 209 | 212 | 212 | 307 |
| 994 | 83 | 88 | 206 | 209 | 212 | 212 | 301 |
| 995 | 83 | 33 | 203 | 209 | 202 | 212 | 302 |
| 996 | 130 | 106 | 205 | 209 | 212 | 212 | 307 |
| 997 | 10 | 98 | 205 | 212 | 201 | 201 | 302 |
| 998 | 10 | 46 | 204 | 209 | 202 | 212 | 308 |
| 999 | 83 | 91 | 206 | 209 | 212 | 212 | 307 |
| 1000 | 90 | 83 | 211 | 202 | 212 | 212 | 303 |
| 1001 | 90 | 86 | 205 | 202 | 201 | 201 | 303 |
| 1002 | 118 | 102 | 201 | 209 | 201 | 201 | 308 |
| 1003 | 10 | 80 | 204 | 212 | 212 | 212 | 306 |
| 1004 | 90 | 23 | 205 | 209 | 202 | 212 | 301 |
| 1005 | 83 | 54 | 212 | 212 | 201 | 201 | 302 |
| 1006 | 90 | 101 | 201 | 201 | 212 | 212 | 308 |
| 1007 | 10 | 91 | 209 | 209 | 212 | 212 | 302 |
| 1008 | 79 | 24 | 204 | 209 | 201 | 201 | 301 |
| 1009 | 10 | 98 | 212 | 212 | 201 | 201 | 307 |
| 1010 | 129 | 71 | 211 | 202 | 202 | 212 | 306 |
| 1011 | 90 | 88 | 210 | 212 | 201 | 201 | 301 |
| 1012 | 83 | 98 | 208 | 212 | 201 | 201 | 301 |
| 1013 | 90 | 13 | 204 | 212 | 201 | 201 | 302 |
| 1014 | 130 | 95 | 204 | 212 | 201 | 201 | 308 |
| 1015 | 129 | 55 | 204 | 212 | 201 | 201 | 302 |
| 1016 | 130 | 101 | 205 | 201 | 202 | 212 | 302 |
| 1017 | 118 | 96 | 201 | 201 | 201 | 201 | 308 |
| 1018 | 90 | 86 | 204 | 202 | 212 | 212 | 304 |
| 1019 | 83 | 101 | 203 | 201 | 201 | 201 | 301 |
| 1020 | 83 | 5 | 205 | 201 | 201 | 201 | 302 |
| 1021 | 118 | 109 | 204 | 202 | 212 | 212 | 303 |
| 1022 | 83 | 38 | 206 | 201 | 201 | 201 | 305 |
| 1023 | 83 | 15 | 211 | 212 | 212 | 212 | 302 |
| 1024 | 130 | 117 | 211 | 212 | 202 | 212 | 302 |
| 1025 | 79 | 91 | 212 | 201 | 201 | 201 | 304 |
| 1026 | 83 | 30 | 210 | 212 | 201 | 201 | 305 |
| 1027 | 130 | 31 | 208 | 202 | 212 | 212 | 305 |
| 1028 | 10 | 22 | 211 | 202 | 201 | 201 | 308 |
| 1029 | 118 | 87 | 206 | 212 | 202 | 212 | 302 |
| 1030 | 130 | 29 | 210 | 201 | 202 | 212 | 306 |
| 1031 | 79 | 61 | 202 | 212 | 202 | 212 | 301 |
| 1032 | 118 | 42 | 205 | 201 | 212 | 212 | 308 |
| 1033 | 118 | 19 | 202 | 212 | 201 | 201 | 308 |
| 1034 | 90 | 39 | 204 | 201 | 212 | 212 | 302 |
| 1035 | 10 | 98 | 208 | 212 | 201 | 201 | 308 |
| 1036 | 118 | 34 | 205 | 212 | 201 | 201 | 304 |
| 1037 | 90 | 78 | 204 | 201 | 201 | 201 | 307 |
| 1038 | 90 | 12 | 207 | 201 | 201 | 201 | 303 |
| 1039 | 129 | 109 | 207 | 209 | 201 | 201 | 304 |
| 1040 | 118 | 105 | 212 | 201 | 202 | 212 | 306 |
| 1041 | 90 | 90 | 205 | 209 | 212 | 212 | 305 |
| 1042 | 90 | 96 | 204 | 202 | 201 | 201 | 308 |
| 1043 | 10 | 48 | 212 | 201 | 202 | 212 | 303 |
| 1044 | 10 | 96 | 209 | 201 | 201 | 201 | 302 |
| 1045 | 129 | 28 | 212 | 212 | 202 | 212 | 302 |
| 1046 | 90 | 53 | 204 | 201 | 201 | 201 | 303 |
| 1047 | 118 | 114 | 212 | 212 | 202 | 212 | 304 |
| 1048 | 130 | 69 | 210 | 202 | 201 | 201 | 303 |
| 1049 | 90 | 100 | 212 | 209 | 212 | 212 | 308 |
| 1050 | 118 | 43 | 212 | 212 | 202 | 212 | 308 |
| 1051 | 130 | 121 | 209 | 202 | 202 | 212 | 308 |
| 1052 | 79 | 127 | 210 | 202 | 201 | 201 | 301 |
| 1053 | 90 | 101 | 211 | 202 | 201 | 201 | 306 |
| 1054 | 83 | 8 | 204 | 209 | 212 | 212 | 301 |
| 1055 | 10 | 109 | 207 | 201 | 201 | 201 | 306 |
| 1056 | 118 | 37 | 204 | 202 | 202 | 212 | 306 |
| 1057 | 79 | 96 | 210 | 202 | 212 | 212 | 305 |
| 1058 | 83 | 17 | 207 | 202 | 201 | 201 | 308 |
| 1059 | 90 | 103 | 205 | 212 | 201 | 201 | 301 |
| 1060 | 83 | 113 | 210 | 202 | 201 | 201 | 307 |
| 1061 | 118 | 108 | 205 | 209 | 212 | 212 | 302 |
| 1062 | 79 | 88 | 206 | 201 | 202 | 212 | 303 |
| 1063 | 90 | 21 | 212 | 212 | 202 | 212 | 301 |
| 1064 | 129 | 101 | 212 | 209 | 202 | 212 | 307 |
| 1065 | 118 | 47 | 203 | 202 | 202 | 212 | 306 |
| 1066 | 10 | 11 | 212 | 201 | 212 | 212 | 306 |
| 1067 | 118 | 63 | 210 | 212 | 202 | 212 | 308 |
| 1068 | 83 | 36 | 212 | 209 | 201 | 201 | 301 |
| 1069 | 10 | 91 | 203 | 201 | 201 | 201 | 306 |
| 1070 | 129 | 85 | 204 | 201 | 212 | 212 | 301 |
| 1071 | 90 | 86 | 210 | 202 | 212 | 212 | 303 |
| 1072 | 130 | 88 | 204 | 201 | 202 | 212 | 306 |
| 1073 | 129 | 40 | 210 | 209 | 202 | 212 | 306 |
| 1074 | 118 | 59 | 206 | 202 | 201 | 201 | 306 |
| 1075 | 90 | 82 | 209 | 201 | 202 | 212 | 302 |
| 1076 | 83 | 124 | 203 | 201 | 201 | 201 | 308 |
| 1077 | 118 | 64 | 212 | 202 | 201 | 201 | 308 |
| 1078 | 118 | 41 | 202 | 209 | 202 | 212 | 302 |
| 1079 | 83 | 86 | 201 | 212 | 212 | 212 | 302 |
| 1080 | 129 | 62 | 205 | 212 | 212 | 212 | 303 |
| 1081 | 10 | 1 | 201 | 212 | 212 | 212 | 308 |
| 1082 | 130 | 123 | 201 | 202 | 201 | 201 | 307 |
| 1083 | 10 | 85 | 212 | 212 | 212 | 212 | 307 |
| 1084 | 118 | 67 | 211 | 202 | 201 | 201 | 302 |
| 1085 | 83 | 84 | 203 | 201 | 212 | 212 | 303 |
| 1086 | 90 | 26 | 209 | 202 | 212 | 212 | 303 |
| 1087 | 118 | 57 | 202 | 202 | 212 | 212 | 308 |
| 1088 | 130 | 88 | 211 | 202 | 201 | 201 | 306 |
| 1089 | 118 | 76 | 202 | 209 | 202 | 212 | 306 |
| 1090 | 130 | 25 | 204 | 202 | 202 | 212 | 306 |
| 1091 | 118 | 130 | 206 | 202 | 202 | 212 | 306 |
| 1092 | 10 | 3 | 203 | 201 | 202 | 212 | 302 |
| 1093 | 83 | 98 | 209 | 212 | 201 | 201 | 307 |
| 1094 | 83 | 4 | 201 | 212 | 201 | 201 | 304 |
| 1095 | 118 | 120 | 201 | 201 | 201 | 201 | 304 |
| 1096 | 90 | 91 | 210 | 212 | 201 | 201 | 308 |
| 1097 | 129 | 88 | 208 | 212 | 212 | 212 | 301 |
| 1098 | 90 | 111 | 204 | 212 | 202 | 212 | 308 |
| 1099 | 130 | 89 | 206 | 202 | 202 | 212 | 306 |
| 1100 | 130 | 115 | 206 | 202 | 201 | 201 | 308 |
| 1101 | 118 | 81 | 210 | 212 | 212 | 212 | 301 |
| 1102 | 10 | 86 | 212 | 201 | 212 | 212 | 301 |
| 1103 | 130 | 16 | 205 | 212 | 201 | 201 | 306 |
| 1104 | 83 | 60 | 202 | 209 | 201 | 201 | 301 |
| 1105 | 118 | 18 | 202 | 201 | 202 | 212 | 306 |
| 1106 | 10 | 32 | 211 | 201 | 212 | 212 | 301 |
| 1107 | 118 | 119 | 212 | 201 | 212 | 212 | 302 |
| 1108 | 90 | 10 | 211 | 201 | 202 | 212 | 308 |
| 1109 | 90 | 7 | 204 | 201 | 201 | 201 | 307 |
| 1110 | 90 | 66 | 201 | 212 | 212 | 212 | 304 |
| 1111 | 79 | 75 | 204 | 209 | 202 | 212 | 302 |
| 1112 | 79 | 110 | 207 | 201 | 201 | 201 | 302 |
| 1113 | 129 | 92 | 204 | 202 | 212 | 212 | 301 |
| 1114 | 129 | 20 | 201 | 209 | 201 | 201 | 305 |
| 1115 | 130 | 85 | 210 | 212 | 202 | 212 | 303 |
| 1116 | 129 | 58 | 202 | 202 | 212 | 212 | 306 |
| 1117 | 79 | 109 | 204 | 209 | 201 | 201 | 303 |
| 1118 | 118 | 49 | 209 | 201 | 201 | 201 | 307 |
| 1119 | 130 | 50 | 212 | 209 | 202 | 212 | 307 |
| 1120 | 129 | 6 | 210 | 201 | 202 | 212 | 306 |
| 1121 | 79 | 97 | 210 | 202 | 201 | 201 | 304 |
| 1122 | 130 | 109 | 212 | 201 | 201 | 201 | 305 |
| 1123 | 90 | 96 | 201 | 212 | 201 | 201 | 304 |
| 1124 | 129 | 68 | 204 | 201 | 212 | 212 | 304 |
| 1125 | 79 | 116 | 205 | 212 | 202 | 212 | 301 |
| 1126 | 118 | 35 | 209 | 201 | 202 | 212 | 301 |
| 1127 | 90 | 98 | 204 | 202 | 212 | 212 | 301 |
| 1128 | 90 | 96 | 211 | 201 | 201 | 201 | 301 |
| 1129 | 79 | 96 | 212 | 201 | 201 | 201 | 301 |
| 1130 | 130 | 56 | 209 | 202 | 202 | 212 | 304 |
| 1131 | 130 | 112 | 212 | 212 | 201 | 201 | 304 |
| 1132 | 83 | 129 | 211 | 202 | 202 | 212 | 302 |
| 1133 | 90 | 122 | 201 | 212 | 201 | 201 | 308 |
| 1134 | 83 | 88 | 209 | 202 | 212 | 212 | 307 |
| 1135 | 10 | 99 | 203 | 201 | 202 | 212 | 307 |
| 1136 | 83 | 128 | 212 | 209 | 201 | 201 | 303 |
| 1137 | 129 | 91 | 201 | 212 | 201 | 201 | 305 |
| 1138 | 130 | 104 | 210 | 202 | 202 | 212 | 307 |
| 1139 | 79 | 126 | 212 | 209 | 202 | 212 | 307 |
| 1140 | 130 | 93 | 205 | 202 | 212 | 212 | 307 |
| 1141 | 118 | 88 | 210 | 202 | 202 | 212 | 306 |
| 1142 | 79 | 79 | 206 | 202 | 212 | 212 | 301 |
| 1143 | 10 | 74 | 210 | 209 | 201 | 201 | 303 |
| 1144 | 130 | 14 | 203 | 201 | 201 | 201 | 308 |
| 1145 | 79 | 86 | 201 | 212 | 202 | 212 | 301 |
| 1146 | 83 | 109 | 207 | 209 | 201 | 201 | 302 |
| 1147 | 10 | 109 | 201 | 202 | 212 | 212 | 301 |
| 1148 | 83 | 44 | 202 | 201 | 201 | 201 | 307 |
| 1149 | 90 | 2 | 201 | 212 | 201 | 201 | 308 |
| 1150 | 10 | 45 | 205 | 202 | 201 | 201 | 303 |
| 1151 | 118 | 73 | 210 | 209 | 202 | 212 | 308 |
| 1152 | 10 | 77 | 204 | 202 | 212 | 212 | 301 |
| 1153 | 83 | 27 | 212 | 212 | 202 | 212 | 306 |
| 1154 | 10 | 118 | 203 | 202 | 212 | 212 | 306 |
| 1155 | 90 | 85 | 204 | 202 | 202 | 212 | 301 |
| 1156 | 118 | 85 | 211 | 202 | 212 | 212 | 301 |
| 1157 | 90 | 94 | 201 | 201 | 212 | 212 | 304 |
| 1158 | 129 | 9 | 212 | 202 | 212 | 212 | 305 |
| 1159 | 10 | 125 | 201 | 212 | 212 | 212 | 308 |
| 1160 | 129 | 72 | 204 | 202 | 202 | 212 | 305 |
| 1161 | 129 | 107 | 204 | 201 | 201 | 201 | 302 |
| 1162 | 10 | 52 | 201 | 202 | 202 | 212 | 306 |
| 1163 | 129 | 51 | 211 | 212 | 201 | 201 | 307 |
| 1164 | 10 | 85 | 204 | 201 | 212 | 212 | 308 |
| 1165 | 90 | 101 | 207 | 212 | 212 | 212 | 301 |

Compounds having the formula:

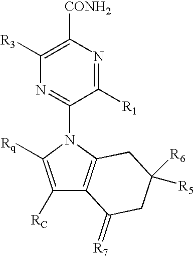

wherein R₁, R₃, R_{C}, R₅, R₆, and R₇ are defined in Table 7:

TABLE 7

| Compound No. | R₁ | R₃ | R_{c} | R_{q} | R₅ | R₆ | R₇ |
|---|---|---|---|---|---|---|---|
| 1166 | 118 | 37 | 204 | 202 | 202 | 212 | 306 |
| 1167 | 90 | 96 | 212 | 209 | 212 | 212 | 307 |
| 1168 | 130 | 117 | 211 | 212 | 202 | 212 | 302 |
| 1169 | 79 | 96 | 210 | 202 | 212 | 212 | 305 |
| 1170 | 79 | 116 | 205 | 212 | 202 | 212 | 301 |
| 1171 | 130 | 31 | 208 | 202 | 212 | 212 | 305 |
| 1172 | 118 | 130 | 206 | 202 | 202 | 212 | 306 |
| 1173 | 118 | 114 | 212 | 212 | 202 | 212 | 304 |
| 1174 | 90 | 90 | 205 | 209 | 212 | 212 | 305 |
| 1175 | 83 | 36 | 212 | 209 | 201 | 201 | 301 |
| 1176 | 90 | 7 | 204 | 201 | 201 | 201 | 307 |
| 1177 | 10 | 98 | 205 | 212 | 201 | 201 | 302 |
| 1178 | 130 | 88 | 211 | 202 | 201 | 201 | 306 |
| 1179 | 10 | 85 | 204 | 201 | 212 | 212 | 308 |
| 1180 | 10 | 98 | 208 | 212 | 201 | 201 | 308 |
| 1181 | 129 | 28 | 212 | 212 | 202 | 212 | 302 |
| 1182 | 90 | 53 | 204 | 201 | 201 | 201 | 303 |
| 1183 | 130 | 95 | 204 | 212 | 201 | 201 | 308 |
| 1184 | 90 | 70 | 205 | 201 | 201 | 201 | 302 |
| 1185 | 83 | 128 | 212 | 209 | 201 | 201 | 303 |
| 1186 | 90 | 23 | 205 | 209 | 202 | 212 | 301 |
| 1187 | 118 | 41 | 202 | 209 | 202 | 212 | 302 |
| 1188 | 129 | 40 | 210 | 209 | 202 | 212 | 306 |
| 1189 | 10 | 109 | 201 | 202 | 212 | 212 | 301 |
| 1190 | 129 | 68 | 204 | 201 | 212 | 212 | 304 |
| 1191 | 79 | 24 | 204 | 209 | 201 | 201 | 301 |
| 1192 | 90 | 91 | 210 | 212 | 201 | 201 | 308 |
| 1193 | 129 | 62 | 205 | 212 | 212 | 212 | 303 |
| 1194 | 83 | 54 | 212 | 212 | 201 | 201 | 302 |
| 1195 | 10 | 98 | 212 | 212 | 201 | 201 | 307 |
| 1196 | 83 | 38 | 206 | 201 | 201 | 201 | 305 |
| 1197 | 83 | 101 | 203 | 201 | 201 | 201 | 301 |
| 1198 | 130 | 85 | 210 | 212 | 202 | 212 | 303 |
| 1199 | 90 | 101 | 211 | 202 | 201 | 201 | 306 |
| 1200 | 118 | 85 | 211 | 202 | 212 | 212 | 301 |
| 1201 | 10 | 80 | 204 | 212 | 212 | 212 | 306 |
| 1202 | 118 | 47 | 203 | 202 | 202 | 212 | 306 |
| 1203 | 10 | 91 | 209 | 209 | 212 | 212 | 302 |
| 1204 | 129 | 92 | 204 | 202 | 212 | 212 | 301 |
| 1205 | 129 | 91 | 201 | 212 | 201 | 201 | 305 |
| 1206 | 118 | 34 | 205 | 212 | 201 | 201 | 304 |
| 1207 | 79 | 86 | 201 | 212 | 202 | 212 | 301 |
| 1208 | 90 | 86 | 204 | 202 | 212 | 212 | 304 |
| 1209 | 83 | 91 | 206 | 209 | 212 | 212 | 307 |
| 1210 | 83 | 8 | 204 | 209 | 212 | 212 | 301 |
| 1211 | 130 | 56 | 209 | 202 | 202 | 212 | 304 |
| 1212 | 90 | 111 | 204 | 212 | 202 | 212 | 308 |
| 1213 | 79 | 127 | 210 | 202 | 201 | 201 | 301 |
| 1214 | 83 | 86 | 201 | 212 | 212 | 212 | 302 |
| 1215 | 130 | 14 | 203 | 201 | 201 | 201 | 308 |
| 1216 | 118 | 87 | 206 | 212 | 202 | 212 | 302 |
| 1217 | 118 | 42 | 205 | 201 | 212 | 212 | 308 |
| 1218 | 90 | 96 | 201 | 212 | 201 | 201 | 304 |
| 1219 | 118 | 64 | 212 | 202 | 201 | 201 | 308 |
| 1220 | 83 | 88 | 209 | 202 | 212 | 212 | 307 |
| 1221 | 130 | 104 | 210 | 202 | 202 | 212 | 307 |
| 1222 | 90 | 101 | 207 | 212 | 212 | 212 | 301 |
| 1223 | 118 | 18 | 202 | 201 | 202 | 212 | 306 |
| 1224 | 79 | 109 | 204 | 209 | 201 | 201 | 303 |
| 1225 | 130 | 89 | 206 | 202 | 202 | 212 | 306 |
| 1226 | 83 | 27 | 212 | 212 | 202 | 212 | 306 |
| 1227 | 90 | 78 | 204 | 201 | 201 | 201 | 307 |
| 1228 | 83 | 33 | 203 | 209 | 202 | 212 | 302 |
| 1229 | 90 | 10 | 211 | 201 | 202 | 212 | 308 |
| 1230 | 118 | 96 | 201 | 201 | 201 | 201 | 308 |
| 1231 | 79 | 65 | 212 | 201 | 212 | 212 | 306 |
| 1232 | 130 | 50 | 212 | 209 | 202 | 212 | 307 |
| 1233 | 129 | 88 | 208 | 212 | 212 | 212 | 301 |
| 1234 | 10 | 46 | 204 | 209 | 202 | 212 | 308 |
| 1235 | 130 | 123 | 201 | 202 | 201 | 201 | 307 |
| 1236 | 83 | 44 | 202 | 201 | 201 | 201 | 307 |
| 1237 | 83 | 17 | 207 | 202 | 201 | 201 | 308 |
| 1238 | 90 | 86 | 210 | 202 | 212 | 212 | 303 |
| 1239 | 79 | 96 | 212 | 201 | 201 | 201 | 301 |
| 1240 | 118 | 81 | 210 | 212 | 212 | 212 | 301 |
| 1241 | 10 | 96 | 209 | 201 | 201 | 201 | 302 |
| 1242 | 118 | 108 | 205 | 209 | 212 | 212 | 302 |
| 1243 | 90 | 82 | 209 | 201 | 202 | 212 | 302 |
| 1244 | 118 | 109 | 204 | 202 | 212 | 212 | 303 |
| 1245 | 10 | 45 | 205 | 202 | 201 | 201 | 303 |
| 1246 | 129 | 107 | 204 | 201 | 201 | 201 | 302 |
| 1247 | 118 | 105 | 212 | 201 | 202 | 212 | 306 |
| 1248 | 90 | 85 | 204 | 202 | 202 | 212 | 301 |
| 1249 | 10 | 48 | 212 | 201 | 202 | 212 | 303 |
| 1250 | 79 | 110 | 207 | 201 | 201 | 201 | 302 |
| 1251 | 79 | 75 | 204 | 209 | 202 | 212 | 302 |
| 1252 | 118 | 88 | 210 | 202 | 202 | 212 | 306 |
| 1253 | 90 | 100 | 212 | 209 | 212 | 212 | 308 |
| 1254 | 129 | 72 | 204 | 202 | 202 | 212 | 305 |
| 1255 | 10 | 1 | 201 | 212 | 212 | 212 | 308 |
| 1256 | 129 | 101 | 212 | 209 | 202 | 212 | 307 |
| 1257 | 90 | 94 | 201 | 201 | 212 | 212 | 304 |
| 1258 | 83 | 84 | 203 | 201 | 212 | 212 | 303 |
| 1259 | 90 | 39 | 204 | 201 | 212 | 212 | 302 |
| 1260 | 83 | 5 | 205 | 201 | 201 | 201 | 302 |
| 1261 | 118 | 120 | 201 | 201 | 201 | 201 | 304 |
| 1262 | 83 | 98 | 208 | 212 | 201 | 201 | 301 |
| 1263 | 83 | 4 | 201 | 212 | 201 | 201 | 304 |
| 1264 | 83 | 129 | 211 | 202 | 202 | 212 | 302 |
| 1265 | 79 | 79 | 206 | 202 | 212 | 212 | 301 |
| 1266 | 90 | 66 | 201 | 212 | 212 | 212 | 304 |
| 1267 | 118 | 19 | 202 | 212 | 201 | 201 | 308 |
| 1268 | 79 | 126 | 212 | 209 | 202 | 212 | 307 |
| 1269 | 83 | 109 | 207 | 209 | 201 | 201 | 302 |
| 1270 | 129 | 55 | 204 | 212 | 201 | 201 | 302 |
| 1271 | 130 | 88 | 204 | 201 | 202 | 212 | 306 |
| 1272 | 10 | 22 | 211 | 202 | 201 | 201 | 308 |
| 1273 | 10 | 52 | 201 | 202 | 202 | 212 | 306 |
| 1274 | 90 | 26 | 209 | 202 | 212 | 212 | 303 |
| 1275 | 118 | 57 | 202 | 202 | 212 | 212 | 308 |
| 1276 | 83 | 124 | 203 | 201 | 201 | 201 | 308 |
| 1277 | 118 | 43 | 212 | 212 | 202 | 212 | 308 |
| 1278 | 83 | 30 | 210 | 212 | 201 | 201 | 305 |
| 1279 | 90 | 13 | 204 | 212 | 201 | 201 | 302 |
| 1280 | 90 | 2 | 201 | 212 | 201 | 201 | 308 |
| 1281 | 130 | 115 | 206 | 202 | 201 | 201 | 308 |
| 1282 | 118 | 76 | 202 | 209 | 202 | 212 | 306 |
| 1283 | 83 | 113 | 210 | 202 | 201 | 201 | 307 |
| 1284 | 90 | 83 | 211 | 202 | 212 | 212 | 303 |
| 1285 | 90 | 96 | 204 | 202 | 201 | 201 | 308 |
| 1286 | 90 | 101 | 201 | 201 | 212 | 212 | 308 |
| 1287 | 130 | 121 | 209 | 202 | 202 | 212 | 308 |
| 1288 | 130 | 25 | 204 | 202 | 202 | 212 | 306 |
| 1289 | 10 | 11 | 212 | 201 | 212 | 212 | 306 |
| 1290 | 130 | 16 | 205 | 212 | 201 | 201 | 306 |
| 1291 | 79 | 88 | 206 | 201 | 202 | 212 | 303 |
| 1292 | 130 | 106 | 205 | 209 | 212 | 212 | 307 |
| 1293 | 118 | 59 | 206 | 202 | 201 | 201 | 306 |
| 1294 | 118 | 73 | 210 | 209 | 202 | 212 | 308 |
| 1295 | 129 | 20 | 201 | 209 | 201 | 201 | 305 |
| 1296 | 130 | 93 | 205 | 202 | 212 | 212 | 307 |
| 1297 | 118 | 35 | 209 | 201 | 202 | 212 | 301 |
| 1298 | 130 | 69 | 210 | 202 | 201 | 201 | 303 |
| 1299 | 10 | 3 | 203 | 201 | 202 | 212 | 302 |
| 1300 | 79 | 61 | 202 | 212 | 202 | 212 | 301 |
| 1301 | 130 | 29 | 210 | 201 | 202 | 212 | 306 |
| 1302 | 10 | 77 | 204 | 202 | 212 | 212 | 301 |
| 1303 | 10 | 109 | 207 | 201 | 201 | 201 | 306 |
| 1304 | 129 | 6 | 210 | 201 | 202 | 212 | 306 |
| 1305 | 79 | 97 | 210 | 202 | 201 | 201 | 304 |
| 1306 | 10 | 91 | 203 | 201 | 201 | 201 | 306 |
| 1307 | 118 | 67 | 211 | 202 | 201 | 201 | 302 |
| 1308 | 118 | 102 | 201 | 209 | 201 | 201 | 308 |
| 1309 | 10 | 32 | 211 | 201 | 212 | 212 | 301 |
| 1310 | 130 | 101 | 205 | 201 | 202 | 212 | 302 |
| 1311 | 129 | 9 | 212 | 202 | 212 | 212 | 305 |
| 1312 | 129 | 85 | 204 | 201 | 212 | 212 | 301 |
| 1313 | 90 | 98 | 204 | 202 | 212 | 212 | 301 |
| 1314 | 129 | 51 | 211 | 212 | 201 | 201 | 307 |
| 1315 | 90 | 103 | 205 | 212 | 201 | 201 | 301 |
| 1316 | 130 | 109 | 212 | 201 | 201 | 201 | 305 |
| 1317 | 10 | 86 | 212 | 201 | 212 | 212 | 301 |
| 1318 | 118 | 49 | 209 | 201 | 201 | 201 | 307 |
| 1319 | 83 | 60 | 202 | 209 | 201 | 201 | 301 |
| 1320 | 90 | 12 | 207 | 201 | 201 | 201 | 303 |
| 1321 | 10 | 85 | 212 | 212 | 212 | 212 | 307 |
| 1322 | 130 | 112 | 212 | 212 | 201 | 201 | 304 |
| 1323 | 10 | 125 | 201 | 212 | 212 | 212 | 308 |
| 1324 | 83 | 15 | 211 | 212 | 212 | 212 | 302 |
| 1325 | 10 | 99 | 203 | 201 | 202 | 212 | 307 |
| 1326 | 118 | 119 | 212 | 201 | 212 | 212 | 302 |
| 1327 | 90 | 88 | 210 | 212 | 201 | 201 | 301 |
| 1328 | 90 | 86 | 205 | 202 | 201 | 201 | 303 |
| 1329 | 83 | 98 | 209 | 212 | 201 | 201 | 307 |
| 1330 | 118 | 63 | 210 | 212 | 202 | 212 | 308 |
| 1331 | 10 | 74 | 210 | 209 | 201 | 201 | 303 |
| 1332 | 129 | 71 | 211 | 202 | 202 | 212 | 306 |
| 1333 | 90 | 122 | 201 | 212 | 201 | 201 | 308 |
| 1334 | 90 | 96 | 211 | 201 | 201 | 201 | 301 |
| 1335 | 83 | 88 | 206 | 209 | 212 | 212 | 301 |
| 1336 | 90 | 21 | 212 | 212 | 202 | 212 | 301 |
| 1337 | 129 | 58 | 202 | 202 | 212 | 212 | 306 |
| 1338 | 79 | 91 | 212 | 201 | 201 | 201 | 304 |
| 1339 | 129 | 109 | 207 | 209 | 201 | 201 | 304 |
| 1340 | 10 | 118 | 203 | 202 | 212 | 212 | 306 |

Compounds having the formula:

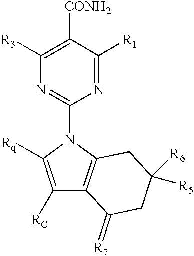

wherein R₁, R₃, R_{C}, R₅, R₆, and R₇ are defined in Table 8:

TABLE 8

| Compound No. | R₁ | R₃ | R_{c} | R_{q} | R₅ | R₆ | R₇ |
|---|---|---|---|---|---|---|---|
| 1341 | 129 | 28 | 212 | 212 | 202 | 212 | 302 |
| 1342 | 129 | 88 | 208 | 212 | 212 | 212 | 301 |
| 1343 | 90 | 82 | 209 | 201 | 202 | 212 | 302 |
| 1344 | 118 | 109 | 204 | 202 | 212 | 212 | 303 |
| 1345 | 90 | 21 | 212 | 212 | 202 | 212 | 301 |
| 1346 | 10 | 77 | 204 | 202 | 212 | 212 | 301 |
| 1347 | 129 | 101 | 212 | 209 | 202 | 212 | 307 |
| 1348 | 83 | 27 | 212 | 212 | 202 | 212 | 306 |
| 1349 | 118 | 42 | 205 | 201 | 212 | 212 | 308 |
| 1350 | 118 | 49 | 209 | 201 | 201 | 201 | 307 |
| 1351 | 90 | 78 | 204 | 201 | 201 | 201 | 307 |
| 1352 | 130 | 14 | 203 | 201 | 201 | 201 | 308 |
| 1353 | 90 | 96 | 204 | 202 | 201 | 201 | 308 |
| 1354 | 10 | 11 | 212 | 201 | 212 | 212 | 306 |
| 1355 | 90 | 122 | 201 | 212 | 201 | 201 | 308 |
| 1356 | 130 | 101 | 205 | 201 | 202 | 212 | 302 |
| 1357 | 90 | 70 | 205 | 201 | 201 | 201 | 302 |
| 1358 | 83 | 44 | 202 | 201 | 201 | 201 | 307 |
| 1359 | 90 | 13 | 204 | 212 | 201 | 201 | 302 |
| 1360 | 118 | 96 | 201 | 201 | 201 | 201 | 308 |
| 1361 | 118 | 35 | 209 | 201 | 202 | 212 | 301 |
| 1362 | 10 | 99 | 203 | 201 | 202 | 212 | 307 |
| 1363 | 118 | 130 | 206 | 202 | 202 | 212 | 306 |
| 1364 | 83 | 15 | 211 | 212 | 212 | 212 | 302 |
| 1365 | 90 | 26 | 209 | 202 | 212 | 212 | 303 |
| 1366 | 90 | 88 | 210 | 212 | 201 | 201 | 301 |
| 1367 | 83 | 128 | 212 | 209 | 201 | 201 | 303 |
| 1368 | 10 | 109 | 201 | 202 | 212 | 212 | 301 |
| 1369 | 130 | 16 | 205 | 212 | 201 | 201 | 306 |
| 1370 | 130 | 115 | 206 | 202 | 201 | 201 | 308 |
| 1371 | 90 | 86 | 205 | 202 | 201 | 201 | 303 |
| 1372 | 83 | 124 | 203 | 201 | 201 | 201 | 308 |
| 1373 | 118 | 63 | 210 | 212 | 202 | 212 | 308 |
| 1374 | 79 | 109 | 204 | 209 | 201 | 201 | 303 |
| 1375 | 118 | 108 | 205 | 209 | 212 | 212 | 302 |
| 1376 | 118 | 81 | 210 | 212 | 212 | 212 | 301 |
| 1377 | 118 | 114 | 212 | 212 | 202 | 212 | 304 |
| 1378 | 130 | 31 | 208 | 202 | 212 | 212 | 305 |
| 1379 | 10 | 32 | 211 | 201 | 212 | 212 | 301 |
| 1380 | 79 | 97 | 210 | 202 | 201 | 201 | 304 |
| 1381 | 90 | 101 | 211 | 202 | 201 | 201 | 306 |
| 1382 | 129 | 72 | 204 | 202 | 202 | 212 | 305 |
| 1383 | 10 | 1 | 201 | 212 | 212 | 212 | 308 |
| 1384 | 130 | 106 | 205 | 209 | 212 | 212 | 307 |
| 1385 | 118 | 85 | 211 | 202 | 212 | 212 | 301 |
| 1386 | 79 | 75 | 204 | 209 | 202 | 212 | 302 |
| 1387 | 90 | 10 | 211 | 201 | 202 | 212 | 308 |
| 1388 | 79 | 127 | 210 | 202 | 201 | 201 | 301 |
| 1389 | 130 | 93 | 205 | 202 | 212 | 212 | 307 |
| 1390 | 129 | 51 | 211 | 212 | 201 | 201 | 307 |
| 1391 | 118 | 105 | 212 | 201 | 202 | 212 | 306 |
| 1392 | 90 | 66 | 201 | 212 | 212 | 212 | 304 |
| 1393 | 83 | 38 | 206 | 201 | 201 | 201 | 305 |
| 1394 | 130 | 109 | 212 | 201 | 201 | 201 | 305 |
| 1395 | 129 | 9 | 212 | 202 | 212 | 212 | 305 |
| 1396 | 129 | 6 | 210 | 201 | 202 | 212 | 306 |
| 1397 | 90 | 96 | 212 | 209 | 212 | 212 | 307 |
| 1398 | 118 | 18 | 202 | 201 | 202 | 212 | 306 |
| 1399 | 10 | 96 | 209 | 201 | 201 | 201 | 302 |
| 1400 | 118 | 43 | 212 | 212 | 202 | 212 | 308 |
| 1401 | 130 | 50 | 212 | 209 | 202 | 212 | 307 |
| 1402 | 83 | 98 | 209 | 212 | 201 | 201 | 307 |
| 1403 | 130 | 117 | 211 | 212 | 202 | 212 | 302 |
| 1404 | 130 | 89 | 206 | 202 | 202 | 212 | 306 |
| 1405 | 130 | 69 | 210 | 202 | 201 | 201 | 303 |
| 1406 | 129 | 92 | 204 | 202 | 212 | 212 | 301 |
| 1407 | 129 | 40 | 210 | 209 | 202 | 212 | 306 |
| 1408 | 10 | 125 | 201 | 212 | 212 | 212 | 308 |
| 1409 | 10 | 48 | 212 | 201 | 202 | 212 | 303 |
| 1410 | 79 | 96 | 212 | 201 | 201 | 201 | 301 |
| 1411 | 90 | 96 | 211 | 201 | 201 | 201 | 301 |
| 1412 | 129 | 91 | 201 | 212 | 201 | 201 | 305 |
| 1413 | 129 | 85 | 204 | 201 | 212 | 212 | 301 |
| 1414 | 118 | 34 | 205 | 212 | 201 | 201 | 304 |
| 1415 | 10 | 85 | 204 | 201 | 212 | 212 | 308 |
| 1416 | 10 | 74 | 210 | 209 | 201 | 201 | 303 |
| 1417 | 90 | 101 | 207 | 212 | 212 | 212 | 301 |
| 1418 | 130 | 85 | 210 | 212 | 202 | 212 | 303 |
| 1419 | 90 | 83 | 211 | 202 | 212 | 212 | 303 |
| 1420 | 118 | 120 | 201 | 201 | 201 | 201 | 304 |
| 1421 | 83 | 98 | 208 | 212 | 201 | 201 | 301 |
| 1422 | 79 | 88 | 206 | 201 | 202 | 212 | 303 |
| 1423 | 118 | 41 | 202 | 209 | 202 | 212 | 302 |
| 1424 | 129 | 71 | 211 | 202 | 202 | 212 | 306 |
| 1425 | 10 | 109 | 207 | 201 | 201 | 201 | 306 |
| 1426 | 10 | 46 | 204 | 209 | 202 | 212 | 308 |
| 1427 | 130 | 121 | 209 | 202 | 202 | 212 | 308 |
| 1428 | 90 | 100 | 212 | 209 | 212 | 212 | 308 |
| 1429 | 83 | 33 | 203 | 209 | 202 | 212 | 302 |
| 1430 | 83 | 88 | 209 | 202 | 212 | 212 | 307 |
| 1431 | 118 | 76 | 202 | 209 | 202 | 212 | 306 |
| 1432 | 10 | 80 | 204 | 212 | 212 | 212 | 306 |
| 1433 | 90 | 39 | 204 | 201 | 212 | 212 | 302 |
| 1434 | 83 | 91 | 206 | 209 | 212 | 212 | 307 |
| 1435 | 90 | 91 | 210 | 212 | 201 | 201 | 308 |
| 1436 | 83 | 17 | 207 | 202 | 201 | 201 | 308 |
| 1437 | 10 | 45 | 205 | 202 | 201 | 201 | 303 |
| 1438 | 130 | 95 | 204 | 212 | 201 | 201 | 308 |
| 1439 | 90 | 23 | 205 | 209 | 202 | 212 | 301 |
| 1440 | 79 | 96 | 210 | 202 | 212 | 212 | 305 |
| 1441 | 90 | 94 | 201 | 201 | 212 | 212 | 304 |
| 1442 | 129 | 55 | 204 | 212 | 201 | 201 | 302 |
| 1443 | 79 | 116 | 205 | 212 | 202 | 212 | 301 |
| 1444 | 129 | 107 | 204 | 201 | 201 | 201 | 302 |
| 1445 | 118 | 64 | 212 | 202 | 201 | 201 | 308 |
| 1446 | 118 | 87 | 206 | 212 | 202 | 212 | 302 |
| 1447 | 79 | 110 | 207 | 201 | 201 | 201 | 302 |
| 1448 | 118 | 88 | 210 | 202 | 202 | 212 | 306 |
| 1449 | 83 | 8 | 204 | 209 | 212 | 212 | 301 |
| 1450 | 90 | 90 | 205 | 209 | 212 | 212 | 305 |
| 1451 | 10 | 98 | 208 | 212 | 201 | 201 | 308 |
| 1452 | 79 | 61 | 202 | 212 | 202 | 212 | 301 |
| 1453 | 83 | 5 | 205 | 201 | 201 | 201 | 302 |
| 1454 | 118 | 102 | 201 | 209 | 201 | 201 | 308 |
| 1455 | 10 | 91 | 203 | 201 | 201 | 201 | 306 |
| 1456 | 118 | 19 | 202 | 212 | 201 | 201 | 308 |
| 1457 | 90 | 85 | 204 | 202 | 202 | 212 | 301 |
| 1458 | 129 | 109 | 207 | 209 | 201 | 201 | 304 |
| 1459 | 83 | 86 | 201 | 212 | 212 | 212 | 302 |
| 1460 | 83 | 4 | 201 | 212 | 201 | 201 | 304 |
| 1461 | 90 | 86 | 204 | 202 | 212 | 212 | 304 |
| 1462 | 130 | 29 | 210 | 201 | 202 | 212 | 306 |
| 1463 | 79 | 86 | 201 | 212 | 202 | 212 | 301 |
| 1464 | 90 | 101 | 201 | 201 | 212 | 212 | 308 |
| 1465 | 83 | 60 | 202 | 209 | 201 | 201 | 301 |
| 1466 | 118 | 73 | 210 | 209 | 202 | 212 | 308 |
| 1467 | 90 | 86 | 210 | 202 | 212 | 212 | 303 |
| 1468 | 129 | 20 | 201 | 209 | 201 | 201 | 305 |
| 1469 | 10 | 52 | 201 | 202 | 202 | 212 | 306 |
| 1470 | 83 | 54 | 212 | 212 | 201 | 201 | 302 |
| 1471 | 118 | 119 | 212 | 201 | 212 | 212 | 302 |
| 1472 | 130 | 25 | 204 | 202 | 202 | 212 | 306 |
| 1473 | 83 | 101 | 203 | 201 | 201 | 201 | 301 |
| 1474 | 10 | 3 | 203 | 201 | 202 | 212 | 302 |
| 1475 | 79 | 126 | 212 | 209 | 202 | 212 | 307 |
| 1476 | 90 | 111 | 204 | 212 | 202 | 212 | 308 |
| 1477 | 79 | 65 | 212 | 201 | 212 | 212 | 306 |
| 1478 | 130 | 112 | 212 | 212 | 201 | 201 | 304 |
| 1479 | 130 | 88 | 204 | 201 | 202 | 212 | 306 |
| 1480 | 83 | 30 | 210 | 212 | 201 | 201 | 305 |
| 1481 | 130 | 88 | 211 | 202 | 201 | 201 | 306 |
| 1482 | 79 | 24 | 204 | 209 | 201 | 201 | 301 |
| 1483 | 118 | 67 | 211 | 202 | 201 | 201 | 302 |
| 1484 | 83 | 88 | 206 | 209 | 212 | 212 | 301 |
| 1485 | 130 | 56 | 209 | 202 | 202 | 212 | 304 |
| 1486 | 129 | 58 | 202 | 202 | 212 | 212 | 306 |
| 1487 | 83 | 84 | 203 | 201 | 212 | 212 | 303 |
| 1488 | 129 | 62 | 205 | 212 | 212 | 212 | 303 |
| 1489 | 90 | 12 | 207 | 201 | 201 | 201 | 303 |
| 1490 | 90 | 7 | 204 | 201 | 201 | 201 | 307 |
| 1491 | 90 | 103 | 205 | 212 | 201 | 201 | 301 |
| 1492 | 10 | 118 | 203 | 202 | 212 | 212 | 306 |
| 1493 | 10 | 22 | 211 | 202 | 201 | 201 | 308 |
| 1494 | 90 | 53 | 204 | 201 | 201 | 201 | 303 |
| 1495 | 90 | 96 | 201 | 212 | 201 | 201 | 304 |
| 1496 | 10 | 91 | 209 | 209 | 212 | 212 | 302 |
| 1497 | 79 | 91 | 212 | 201 | 201 | 201 | 304 |
| 1498 | 90 | 98 | 204 | 202 | 212 | 212 | 301 |
| 1499 | 129 | 68 | 204 | 201 | 212 | 212 | 304 |
| 1500 | 130 | 104 | 210 | 202 | 202 | 212 | 307 |
| 1501 | 10 | 86 | 212 | 201 | 212 | 212 | 301 |
| 1502 | 10 | 98 | 205 | 212 | 201 | 201 | 302 |
| 1503 | 130 | 123 | 201 | 202 | 201 | 201 | 307 |
| 1504 | 83 | 113 | 210 | 202 | 201 | 201 | 307 |
| 1505 | 118 | 37 | 204 | 202 | 202 | 212 | 306 |
| 1506 | 83 | 109 | 207 | 209 | 201 | 201 | 302 |
| 1507 | 83 | 129 | 211 | 202 | 202 | 212 | 302 |
| 1508 | 118 | 59 | 206 | 202 | 201 | 201 | 306 |
| 1509 | 10 | 98 | 212 | 212 | 201 | 201 | 307 |
| 1510 | 79 | 79 | 206 | 202 | 212 | 212 | 301 |
| 1511 | 118 | 47 | 203 | 202 | 202 | 212 | 306 |
| 1512 | 10 | 85 | 212 | 212 | 212 | 212 | 307 |
| 1513 | 90 | 2 | 201 | 212 | 201 | 201 | 308 |
| 1514 | 83 | 36 | 212 | 209 | 201 | 201 | 301 |
| 1515 | 118 | 57 | 202 | 202 | 212 | 212 | 308 |

Biological Evaluation

### Example 4

### Cell Proliferation Assays

A panel of cancer cell lines is obtained from the DCTP Tumor Repository, National Cancer Institute (Frederick, Md.) or ATCC (Rockville, Md.). Cell cultures are maintained in Hyclone RPMI 1640 medium (Logan, Utah) supplemented with 10% fetal bovine serum and 20 mM HEPES buffer, final pH 7.2, at 37° C. with a 5% CO₂ atmosphere. Cultures are maintained at sub-confluent densities. Human umbilical vein endothelial cells (HUVEC) are purchased from Clonetics, a division of Cambrex (Walkersville, Md.). Cultures are established from cryopreserved stocks using Clonetics EGM-2 medium supplemented with 20 mM HEPES, final pH 7.2, at 37° C. with a 5% CO₂ atmosphere.

For proliferation assays, cells are seeded with the appropriate medium into 96 well plates at 1,000-2,500 cells per well, depending on the cell line, and are incubated overnight. The following day, test compound, DMSO solution (negative control), or Actinomycin D (positive control) is added to the appropriate wells as 10× concentrated stocks prepared in phosphate buffered saline. The cell plates are then incubated for an additional 2-5 days, depending on the cell line, to allow proliferation to occur. To measure cell density, 50 μL of WST-1 solution (Roche Applied Science, IN) diluted 1:5 in phosphate buffered saline is added to each well, and the cells incubated for an additional 1-5 hrs., again depending on the cell line. Optical density is determined for each well at 450 nM using a Tecan GeniosPro plate reader (RTP, NC). The percentage of cell growth is determined by comparing the cell growth in the presence of test compounds to the cells treated with DMSO vehicle (control, 100% growth) and cells treated with Actinomycin D (10 μM, 0% growth).

Immediately after the WST-1 determination, the medium is removed from the PC-3, NCI-H460 and HUVEC cell lines, and the plates stored at −80° C. Using these assay plates, relative amounts of DNA in each well are determined using the Cyquant DNA assay kit from R&D Systems (Eugene, Oreg.) following the manufacturer's directions. Results for each compound treatment are compared to DMSO vehicle control (100%) and 10 μM Actinomycin D treated cells (0%).

Compounds useful in the methods of the invention generally have inhibitory IC₅₀ values for at least one of these cell lines below 50 μM.

### Example 5

### Determination of Affinity for HSP-90

Affinity of test compounds for HSP-90 is determined as follows: Protein mixtures obtained from a variety of organ tissues (for example: spleen, liver and lung) are reversibly bound to a purine affinity column to capture purine-binding proteins, especially HSP-90. The purine affinity column is washed several times, and then eluted with 20 μM, 100 μM, and 500 μM of test compound. Compounds of Formula I elute HP-90 in a dose-dependent manner vs. a control elution using dimethylsulfoxide. The elution profile of Formula I compounds is determined by 1-dimensional SDS polyacrylamide gel electrophoresis. Gels are stained with a fluorescent stain such as sypro ruby (a highly sensitive fluorescent protein stain that can readily detect less than 1 fmol of total protein, i.e., less than 0.04 ng for a 40 kDa protein) or silver nitrate. The gels are imaged using a standard flat bed gel imager and the amount of protein estimated by densitometry. The percent of HSP-90 protein eluted from the column at each concentration is determined and IC₅₀ values are calculated from these estimates. Analysis of the gels indicates that compounds of the invention are inhibitors of HSP-90 (heat shock protein 90) having IC₅₀ values within the range of 1 μM to 50 μM.

The invention and the manner and process of making and using it, are now described in such full, clear, concise and exact terms as to enable any person skilled in the art to which it pertains, to make and use the same. It is to be understood that the foregoing describes preferred embodiments of the invention and that modifications may be made therein without departing from the spirit or scope of the invention as set forth in the claims. To particularly point out and distinctly claim the subject matter regarded as invention, the following claims conclude this specification.

What is claimed is:

1. A compound according to the formula, 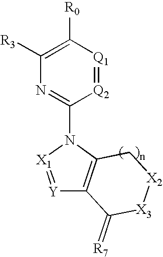
and pharmaceutically acceptable salts thereof, wherein
Q₁ and Q₂ are independently N or CR₁, wherein one and only one of Q₁ and Q₂ must be N;
R₁ and R₀ are independently hydrogen, halogen, hydroxyl, cyano, nitro, amino, mono- or di-(C₁-C₁₀)alkylamino, carboxamido, —NHaryl, —NHheteroaryl, C₁-C₁₀ haloalkyl, C₁-C₁₀ alkyl, C₃-C₁₀ cycloalkyl, C₃-C₁₀ heterocycloalkyl, aryl, heteroaryl, or a group of the formula 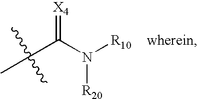
X₄ is O, NH, NOH, or S; and
R₁₀ and R₂₀ are independently H, OH, C₁-C₁₀ haloalkyl, C₁-C₁₀alkyl, C₃-C₁₀cycloalkyl, C₃-C₁₀ heterocycloalkyl, aryl, or heteroaryl;
wherein each alkyl, aryl, cycloalkyl, heterocycloalkyl, and heteroaryl group is optionally substituted with one to four groups which are each independently C₁-C₆ alkyl, C₁-C₆ alkoxy, halogen, carboxy, oxo, amino, mono- or di-(C₁-C₆)alkylamino, cyano, nitro, halo(C₁-C₆)alkyl, halo(C₁-C₆)alkoxy, carboxamide, heterocycloalkyl, aryl, or heteroaryl, wherein
the aryl and heteroaryl groups are optionally substituted with from one to four groups what are each independently C₁-C₆ alkyl, C₁-C₆ alkoxy, halogen, hydroxy, amino, mono- or di-(C₁-C₆)alkylamino, halo(C₁-C₆)alkyl, or carboxamide;
R₃ is (a) H; (b) halo; or
(c) a C₁-C₁₅ alkyl group where up to six of the carbon atoms in said alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other, wherein
R₂₂ is
(i) heteroaryl,
(ii) aryl,
(iii) saturated or unsaturated C₃-C₁₀ cycloalkyl, or
(iv) saturated or unsaturated C₃-C₁₀ heterocycloalkyl, wherein
each aryl, heteroaryl, saturated or unsaturated cycloalkyl, or saturated or unsaturated heterocycloalkyl, independently, is optionally substituted with at least one group, which independently is hydroxy, halo, amino, cyano, carboxy, carboxamido, nitro, oxo, —S—(C₁-C₆)alkyl, —SO₂—(C₁-C₆)alkyl, —SO₂-aryl, —SO—(C₁-C₆)alkyl, —SO-aryl, —SO₂NH₂, —SO₂NH—(C₁-C₆)alkyl, —SO₂NH-aryl, (C₁-C₆)alkoxy, or mono- or di-(C₁-C₁₀)alkylamino; and
each R₂₂ is optionally fused to a C₆-C₁₀ aryl group, C₅-C₈ saturated cyclic group, or a C₅-C₁₀ heterocycloalkyl group;
wherein each (c) is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S—(C₁-C₆)alkyl, —SO₂—(C₁-C₆)alkyl, —SO₂NH₂, —SO₂NH—(C₁-C₆)alkyl, —SO₂NH-aryl, —SO₂-aryl, —SO—(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, or R₂₃, wherein
R₂₃ is
(1) heteroaryl,
(2) aryl,
(3) saturated or unsaturated C₅-C₁₀ cycloalkyl, or
(4) saturated or unsaturated C₅-C₁₀ heterocycloalkyl, and
the R₂₃ groups are optionally substituted at least one group which is independently hydroxy, oxo, halo, amino, cyano, nitro, —SH, —S—(C₁-C₆)alkyl, —SO₂—(C₁-C₆)alkyl, —SO₂-aryl, —SO—(C₁-C₆)alkyl, —SO-aryl, —SO₂NH₂, —SO₂NH—(C₁-C₆)alkyl, —SO₂NH-aryl, (C₁-C₆)alkoxy, or mono- or di-(C₁-C₁₀)alkylamino;
R₇ is O, S, or NR_{7′}, wherein
R_{7′} is H, —OH, —NH₂, —NHR₂₂, —NH—(C₁-C₆ alkyl), —O—(C₀-C₆)alkyl-R₂₂, or —(C₁-C₆ alkoxy optionally substituted with carboxy);
X₁ is N or CR_{C};
each R_{C} independently is hydrogen, halogen, cyano, nitro, C₁-C₁₀ alkyl, C₂-C₁₀ alkenyl, C₂-C₁₀ alkynyl, C₁-C₁₀ haloalkyl, C₃-C₇ cycloalkyl, C₃-C₇ cycloalkyl(C₁-C₁₀)alkyl, heterocycloalkyl, aryl, or heteroaryl, wherein
each alkyl, aryl, cycloalkyl, heterocycloalkyl, and heteroaryl group is optionally substituted with from 1-4 groups that are independently C₁-C₆ alkyl, C₁-C₆ alkoxy, halogen, hydroxy, amino, mono- or di-(C₁-C₆)alkylamino, cyano, nitro, halo(C₁-C₆)alkyl, halo(C₁-C₆)alkoxy, carboxamide, heterocycloalkyl, aryl, or heteroaryl, wherein
the aryl and heteroaryl groups are optionally substituted with from 1-4 groups that are independently C₁-C₆ alkyl, C₁-C₆ alkoxy, halogen, hydroxy, amino, mono- or di-(C₁-C₆)alkylamino, halo(C₁-C₆)alkyl, or carboxamide;
Y is N or CR_{C};
X₂ and X₃ are independently C(R₅)(R₆), O, N(R₅), or S(O)ₚ wherein
each R₅ and R₆ is independently hydrogen, C₁-C₆ alkyl, or mono- or di-(C₁-C₆)alkylamino(C₁-C₆)alkyl or R₅ and R₆ together with the carbon to which they are attached form a 3-8 membered cycloalkyl or heterocycloalkyl ring; and
p is 0, 1, or 2; and
n is 0, 1, 2, 3, or 4.

2. A compound according to claim 1, wherein
R₀ is cyano, hydroxyl, nitro, amino, mono- or di-(C₁-C₁₀) alkylamino, or a group of the formula 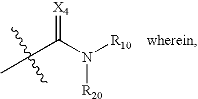
X₄ is O, NH, NOH, or S; and
R₁₀ and R₂₀ are independently H, OH, C₁-C₁₀ haloalkyl, or C₁-C₁₀alkyl.

3. A compound according to claim 2, wherein
R₀ is cyano or —CONH₂.

4. A compound according to claim 3, wherein
R₀ is cyano.

5. A compound according to claim 3, wherein
R₀ is —CONH₂.

6. A compound according to claim 1, wherein
R₃ is hydrogen, halo, or -Z₁R_{Z1}, wherein
Z₁ is —O—, —NH—, —S(O)ₚ—, or —S(O)₂NH—, wherein
p is 0, 1 or 2; and
R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S—(C₁-C₆)alkyl, —SO₂—(C₁-C₆)alkyl, —SO₂NH₂, —SO₂NH—(C₁-C₆)alkyl, —SO₂NH-aryl, —SO₂-aryl, —SO—(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, or R₂₃.

7. A compound according to claim 6, wherein
R₃ is hydrogen, halo, or -Z₁R_{Z1}, wherein
Z₁ is —O— or —NH—; and
R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S—(C₁-C₆)alkyl, —SO₂—(C₁-C₆)alkyl, —SO₂NH₂, —SO₂NH—(C₁-C₆)alkyl, —SO₂NH-aryl, —SO₂-aryl, —SO—(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, or R₂₃.

8. A compound according to claim 7, wherein
R₃ is hydrogen, halo, or —N(H)R_{Z1}, wherein
R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, or R₂₃.

9. A compound according to claim 1, wherein X₁ is N.

10. A compound according to claim 1, wherein Y is N.

11. A compound according to claim 1, wherein X₁ is CR_{C}.

12. A compound according to claim 1, wherein Y is CR_{C}.

13. A compound according to claim 9, wherein
Y is CR_{C}.

14. A compound according to claim 13, wherein
R_{C} is hydrogen, halogen, C₁-C₁₀ alkyl, C₁-C₁₀ haloalkyl, C₃-C₇ cycloalkyl, or C₃-C₇ cycloalkyl(C₁-C₁₀)alkyl.

15. A compound according to claim 13, wherein
R_{C} is hydrogen, halogen, methyl, ethyl, fluoromethyl, difluoromethyl, trifluoromethyl, cyclopropyl, or cyclopropylmethyl.

16. A compound according to claim 11, wherein
Y is CR_{C}, wherein
each R_{C} is independently hydrogen, halogen, C₁-C₁₀ alkyl, C₁-C₁₀ haloalkyl, C₃-C₇ cycloalkyl, or C₃-C₇ cycloalkyl(C₁-C₁₀)alkyl.

17. A compound according to claim 16, wherein
each R_{C} is independently hydrogen, halogen, methyl, ethyl, fluoromethyl, difluoromethyl, trifluoromethyl, cyclopropyl, or cyclopropylmethyl.

18. A compound according to claim 1, wherein
Q₁ is N; and
Q₂ is CR₁, wherein
R₁ is hydrogen, halogen, amino, C₁-C₁₀ alkyl, or C₁-C₁₀ haloalkyl.

19. A compound according to claim 1, wherein
Q₂ is N; and
Q₁ is CR₁, wherein
R₁ is hydrogen, halogen, amino, C₁-C₁₀ alkyl, or C₁-C₁₀ haloalkyl.

20. A compound according to claim 1, one of the formulas,

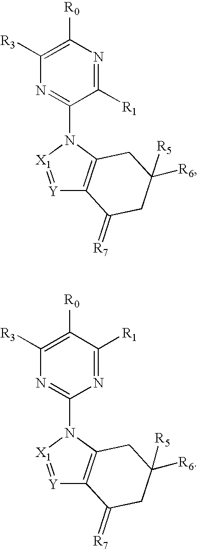

21. A compound according to claim 20, of one of the formulas,

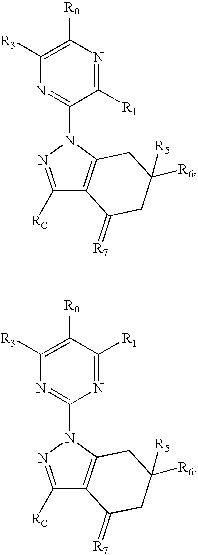

22. A compound according to claim 21, wherein
R₀ is cyano, hydroxyl, nitro, amino, mono- or di-(C₁-C₁₀)alkylamino, or a group of the formula 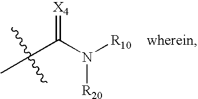
X₄ is O, NH, NOH, or S; and
R₁₀ and R₂₀ are independently H, OH, C₁-C₁₀ haloalkyl, or C₁-C₁₀alkyl.

23. A compound according to claim 22, wherein
R₀ is cyano or —CONH₂.

24. A compound according to claim 23, wherein
R₀ is cyano.

25. A compound according to claim 23, wherein
R₀ is —CONH₂.

26. A compound according to claim 21, wherein
R₃ is hydrogen, halo, or -Z₁R_{Z1}, wherein
Z₁ is —O—, —NH—, —S(O)ₚ—, or —S(O)₂NH—, wherein
p is 0, 1 or 2; and
R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S—(C₁-C₆)alkyl, —SO₂—(C₁-C₆)alkyl, —SO₂NH₂, —SO₂NH—(C₁-C₆)alkyl, —SO₂NH-aryl, —SO₂-aryl, —SO—(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, or R₂₃.

27. A compound according to claim 26, wherein
R₃ is hydrogen, halo, or -Z₁R_{Z1}, wherein
Z₁ is —O— or —NH—; and
R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S—(C₁-C₆)alkyl, —SO₂—(C₁-C₆)alkyl, —SO₂NH₂, —SO₂NH—(C₁-C₆)alkyl, —SO₂NH-aryl, —SO₂-aryl, —SO—(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, or R₂₃.

28. A compound according to claim 27, wherein
R₃ is hydrogen, halo, or —N(H)R_{Z1}, wherein
R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, or R₂₃.

29. A compound according to claim 21, wherein
R_{C} is hydrogen, halogen, C₁-C₁₀ alkyl, C₁-C₁₀ haloalkyl, C₃-C₇ cycloalkyl, or C₃-C₇ cycloalkyl(C₁-C₁₀)alkyl.

30. A compound according to claim 29, wherein
R_{C} is hydrogen, halogen, methyl, ethyl, fluoromethyl, difluoromethyl, trifluoromethyl, cyclopropyl, or cyclopropylmethyl.

31. A compound according to claim 21, wherein
R₁ is hydrogen, halogen, amino, C₁-C₁₀ alkyl, or C₁-C₁₀ haloalkyl.

32. A compound according to claim 1, of one of the formulas,

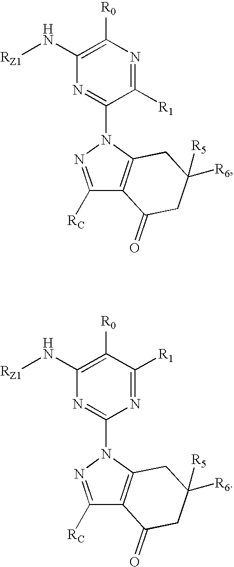

33. A compound according to claim 32, wherein
R₀ is cyano, hydroxyl, nitro, amino, mono- or di-(C₁-C₁₀) alkylamino, or a group of the formula 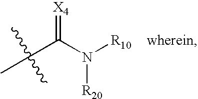
X₄ is O, NH, NOH, or S; and
R₁₀ and R₂₀ are independently H, OH, C₁-C₁₀ haloalkyl, or C₁-C₁₀alkyl.

34. A compound according to claim 33, wherein
R₀ is cyano or —CONH₂.

35. A compound according to claim 34, wherein
R₀ is cyano.

36. A compound according to claim 34, wherein
R₀ is —CONH₂.

37. A compound according to claim 32, wherein
R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S—(C₁-C₆)alkyl, —SO₂—(C₁-C₆)alkyl, —SO₂NH₂, —SO₂NH—(C₁-C₆)alkyl, —SO₂NH-aryl, —SO₂-aryl, —SO—(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, or R₂₃.

38. A compound according to claim 37, wherein
R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, or R₂₃.

39. A compound according to claim 32, wherein
R_{C} is hydrogen, halogen, C₁-C₁₀ alkyl, C₁-C₁₀ haloalkyl, C₃-C₇ cycloalkyl, or C₃-C₇ cycloalkyl(C₁-C₁₀)alkyl.

40. A compound according to claim 39, wherein
R_{C} is hydrogen, halogen, methyl, ethyl, fluoromethyl, difluoromethyl, trifluoromethyl, cyclopropyl, or cyclopropylmethyl.

41. A compound according to claim 32, wherein
R₁ is hydrogen, halogen, amino, C₁-C₁₀ alkyl, or C₁-C₁₀ haloalkyl.

42. A compound according to claim 1, of one of the formulas,

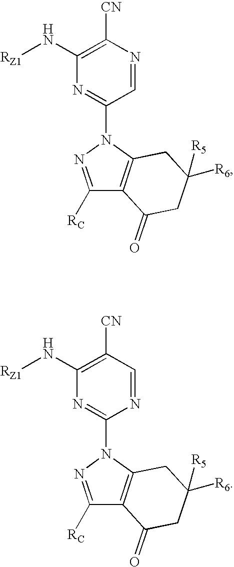

43. A compound according to claim 42, wherein
R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S—(C₁-C₆)alkyl, —SO₂—(C₁-C₆)alkyl, —SO₂NH₂, —SO₂NH—(C₁-C₆)alkyl, —SO₂NH-aryl, —SO₂-aryl, —SO—(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, or R₂₃.

44. A compound according to claim 43, wherein
R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, or R₂₃.

45. A compound according to claim 42, wherein
R_{C} is hydrogen, halogen, C₁-C₁₀ alkyl, C₁-C₁₀ haloalkyl, C₃-C₇ cycloalkyl, or C₃-C₇ cycloalkyl(C₁-C₁₀)alkyl.

46. A compound according to claim 45, wherein
R_{C} is hydrogen, halogen, methyl, ethyl, fluoromethyl, difluoromethyl, trifluoromethyl, cyclopropyl, or cyclopropylmethyl.

47. A compound according to claim 42, wherein
R₁ is hydrogen, halogen, amino, C₁-C₁₀ alkyl, or C₁-C₁₀ haloalkyl.

48. A compound according to claim 1, of one of the formulas,

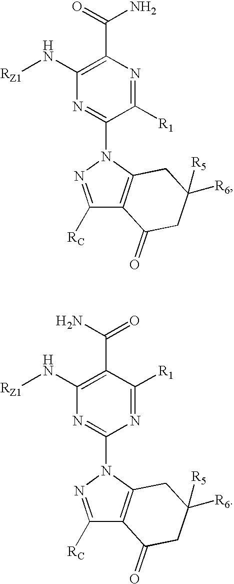

49. A compound according to claim 48, wherein
R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S—(C₁-C₆)alkyl, —SO₂—(C₁-C₆)alkyl, —SO₂NH₂, —SO₂NH—(C₁-C₆)alkyl, —SO₂NH-aryl, —SO₂-aryl, —SO—(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, or R₂₃.

50. A compound according to claim 49, wherein
R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, or R₂₃.

51. A compound according to claim 48, wherein
R_{C} is hydrogen, halogen, C₁-C₁₀ alkyl, C₁-C₁₀ haloalkyl, C₃-C₇ cycloalkyl, or C₃-C₇ cycloalkyl(C₁-C₁₀)alkyl.

52. A compound according to claim 51, wherein
R_{C} is hydrogen, halogen, methyl, ethyl, fluoromethyl, difluoromethyl, trifluoromethyl, cyclopropyl, or cyclopropylmethyl.

53. A compound according to claim 48, wherein
R₁ is hydrogen, halogen, amino, C₁-C₁₀ alkyl, or C₁-C₁₀ haloalkyl.

54. A compound according to claim 53, wherein R₁ is H.

55. A compound according to claim 20, of one of the formulas,

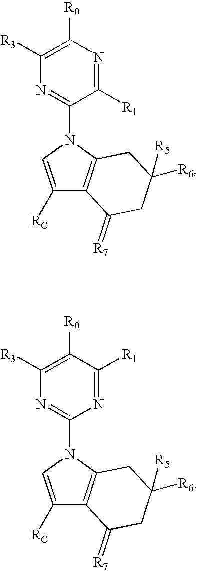

56. A compound according to claim 55, wherein
R₀ is cyano, hydroxyl, nitro, amino, mono- or di-(C₁-C₁₀)alkylamino, or a group of the formula 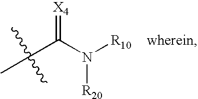
X₄ is O, NH, NOH, or S; and
R₁₀ and R₂₀ are independently H, OH, C₁-C₁₀ haloalkyl, or C₁-C₁₀alkyl.

57. A compound according to claim 56, wherein
R₀ is cyano or —CONH₂.

58. A compound according to claim 57, wherein
R₀ is cyano.

59. A compound according to claim 57, wherein
R₀ is —CONH₂.

60. A compound according to claim 55, wherein
R₃ is hydrogen, halo, or -Z₁R_{Z1}, wherein
Z₁ is —O—, —NH—, —S(O)ₚ—, or —S(O)₂NH—, wherein
p is 0, 1 or 2; and
R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S—(C₁-C₆)alkyl, —SO₂—(C₁-C₆)alkyl, —SO₂NH₂, —SO₂NH—(C₁-C₆)alkyl, —SO₂NH-aryl, —SO₂-aryl, —SO—(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, or R₂₃.

61. A compound according to claim 60, wherein
R₃ is hydrogen, halo, or -Z₁R_{Z1}, wherein
Z₁ is —O— or —NH—; and
R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S—(C₁-C₆)alkyl, —SO₂—(C₁-C₆)alkyl, —SO₂NH₂, —SO₂NH—(C₁-C₆)alkyl, —SO₂NH-aryl, —SO₂-aryl, —SO—(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, or R₂₃.

62. A compound according to claim 61, wherein
R₃ is hydrogen, halo, or —N(H)R_{Z1}, wherein
R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, or R₂₃.

63. A compound according to claim 55, wherein
R_{C} is hydrogen, halogen, C₁-C₁₀ alkyl, C₁-C₁₀ haloalkyl, C₃-C₇ cycloalkyl, or C₃-C₇ cycloalkyl(C₁-C₁₀)alkyl.

64. A compound according to claim 63, wherein
R_{C} is hydrogen, halogen, methyl, ethyl, fluoromethyl, difluoromethyl, trifluoromethyl, cyclopropyl, or cyclopropylmethyl.

65. A compound according to claim 55, wherein
R₁ is hydrogen, halogen, amino, C₁-C₁₀ alkyl, or C₁-C₁₀ haloalkyl.

66. A compound according to claim 1, of one of the formulas,

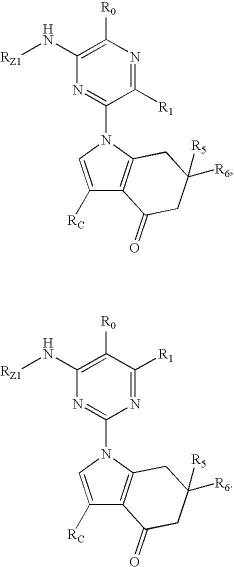

67. A compound according to claim 66, wherein
R₀ is cyano, hydroxyl, nitro, amino, mono- or di-(C₁-C₁₀) alkylamino, or a group of the formula 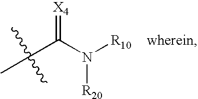
X₄ is O, NH, NOH, or S; and
R₁₀ and R₂₀ are independently H, OH, C₁-C₁₀ haloalkyl, or C₁-C₁₀alkyl.

68. A compound according to claim 67, wherein
R₀ is cyano or —CONH₂.

69. A compound according to claim 68, wherein
R₀ is cyano.

70. A compound according to claim 68, wherein
R₀ is —CONH₂.

71. A compound according to claim 66, wherein
R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S—(C₁-C₆)alkyl, —SO₂—(C₁-C₆)alkyl, —SO₂NH₂, —SO₂NH—(C₁-C₆)alkyl, —SO₂NH-aryl, —SO₂-aryl, —SO—(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, or R₂₃.

72. A compound according to claim 71, wherein
R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, or R₂₃.

73. A compound according to claim 66, wherein
R_{C} is hydrogen, halogen, C₁-C₁₀ alkyl, C₁-C₁₀ haloalkyl, C₃-C₇ cycloalkyl, or C₃-C₇ cycloalkyl(C₁-C₁₀)alkyl.

74. A compound according to claim 73, wherein
R_{C} is hydrogen, halogen, methyl, ethyl, fluoromethyl, difluoromethyl, trifluoromethyl, cyclopropyl, or cyclopropylmethyl.

75. A compound according to claim 66, wherein
R₁ is hydrogen, halogen, amino, C₁-C₁₀ alkyl, or C₁-C₁₀ haloalkyl.

76. A compound according to claim 1, of one of the formulas,

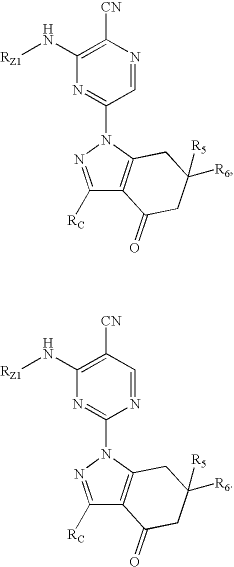

77. A compound according to claim 76, wherein
R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S—(C₁-C₆)alkyl, —SO₂—(C₁-C₆)alkyl, —SO₂NH₂, —SO₂NH—(C₁-C₆)alkyl, —SO₂NH-aryl, —SO₂-aryl, —SO—(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, or R₂₃.

78. A compound according to claim 77, wherein
R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, or R₂₃.

79. A compound according to claim 76, wherein
R_{C} is hydrogen, halogen, C₁-C₁₀ alkyl, C₁-C₁₀ haloalkyl, C₃-C₇ cycloalkyl, or C₃-C₇ cycloalkyl(C₁-C₁₀)alkyl.

80. A compound according to claim 79, wherein
R_{C} is hydrogen, halogen, methyl, ethyl, fluoromethyl, difluoromethyl, trifluoromethyl, cyclopropyl, or cyclopropylmethyl.

81. A compound according to claim 76, wherein
R₁ is hydrogen, halogen, amino, C₁-C₁₀ alkyl, or C₁-C₁₀ haloalkyl.

82. A compound according to claim 1, of one of the formulas,

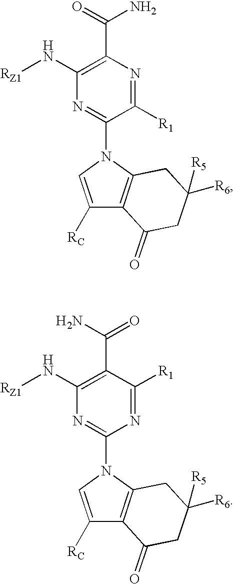

83. A compound according to claim 82, wherein
R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S—(C₁-C₆)alkyl, —SO₂—(C₁-C₆)alkyl, —SO₂NH₂, —SO₂NH—(C₁-C₆)alkyl, —SO₂NH-aryl, —SO₂-aryl, —SO—(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, or R₂₃.

84. A compound according to claim 83, wherein
R_{Z1} is a C₁-C₁₄ alkyl group where up to five of the carbon atoms in the alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, or R₂₃.

85. A compound according to claim 82, wherein
R_{C} is hydrogen, halogen, C₁-C₁₀ alkyl, C₁-C₁₀ haloalkyl, C₃-C₇ cycloalkyl, or C₃-C₇ cycloalkyl(C₁-C₁₀)alkyl.

86. A compound according to claim 85, wherein
R_{C} is hydrogen, halogen, methyl, ethyl, fluoromethyl, difluoromethyl, trifluoromethyl, cyclopropyl, or cyclopropylmethyl.

87. A compound according to claim 82, wherein
R₁ is hydrogen, halogen, amino, C₁-C₁₀ alkyl, or C₁-C₁₀ haloalkyl.

88. A compound according to claim 87, wherein R₁ is H.

89. A pharmaceutical composition comprising at least one compound or salt according to claim 1 and a pharmaceutically acceptable solvent, carrier, excipient, adjuvant or a combination thereof.

90. A method of treating cancer, inflammation, or arthritis comprising administering to a patient in need of such treatment a therapeutically effective amount of a compound or salt of claim 1.

91. A method for treating a subject suffering from a disease or disorder of proteins that are either client proteins for HSP-90 or indirectly affect its client proteins, wherein disorder is selected from the group of inflammatory diseases, infections, autoimmune disorders, stroke, ischemia, cardiac disorders, neurological disorders, fibrogenetic disorders, proliferative disorders, tumors, leukemias, neoplasms, cancers, carcinomas, metabolic diseases, malignant disease, scleroderma, polymyositis, systemic lupus, rheumatoid arthritis, liver cirrhosis, keloid formation, interstitial nephritis, pulmonary fibrosis, and sepsis, comprising administering to a subject in need of such treatment a therapeutically effective amount of a compound or salt of claim 1.

92. A method of reducing the level of infection in a subject where the infection is caused by an organism selected from Plasmodium species, the method comprising administering to an infected subject an effective amount of a compound or salt according to claim 1.

93. A method for treating a fungal infection in a patient in need of such treatment, comprising administering an effective amount of a compound or salt according to claim 1 and an optional anti-fungal agent or drug.

94. A method according to claim 90, for the treatment of cancer and further comprising administration of (a) at least one additional anti-cancer agent or composition or (b) radiation therapy.

95. A method of treating a patient suffering from a viral infection comprising administering to the patient a therapeutically effective amount of a compound of claim 1.

96. A compound according to claim 1 which is
4-(2-Methoxy-ethylamino)-2-(3,6,6-trimethyl-4-oxo-4,5,6,7-tetrahydro-indazol-1-yl)-pyrimidine-5-carbonitrile; or
4-(2-Methoxy-ethylamino)-2-(3,6,6-trimethyl-4-oxo-4,5,6,7-tetrahydro-indazol-1-yl)-pyrimidine-5-carboxylic acid amide.

97. A compound according to claim 1 which is
4-(2-methoxyethylamino)-2-(2,3,6,6-tetramethyl-4-oxo-4,5,6,7-tetrahydro-1H-indol-1-yl)pyrimidine-5-carboxamide;
4-(cyclopentylamino)-2-(2,3,6,6-tetramethyl-4-oxo-4,5,6,7-tetrahydro-1H-indol-1-yl)pyrimidine-5-carboxamide;
4-(cyclopropylmethylamino)-2-(2,3,6,6-tetramethyl-4-oxo-4,5,6,7-tetrahydro-1H-indol-1-yl)pyrimidine-5-carboxamide;
3-(1,3-dimethoxypropan-2-ylamino)-5-(2,3,6,6-tetramethyl-4-oxo-4,5,6,7-tetrahydro-1H-indol-1-yl)pyrazine-2-carboxamide;
3-(2-aminocyclohexylamino)-5-(2,3,6,6-tetramethyl-4-oxo-4,5,6,7-tetrahydro-1H-indol-1-yl)pyrazine-2-carboxamide;
3-(cyclopent-3-enylamino)-5-(2,3,6,6-tetramethyl-4-oxo-4,5,6,7-tetrahydro-1H-indol-1-yl)pyrazine-2-carboxamide;
4-(tetrahydrofuran-3-ylamino)-2-(3,6,6-trimethyl-4-oxo-4,5,6,7-tetrahydro-1H-indol-1-yl)pyrimidine-5-carboxamide;
4-(tetrahydro-2H-pyran-4-ylamino)-2-(3,6,6-trimethyl-4-oxo-4,5,6,7-tetrahydro-1H-indol-1-yl)pyrimidine-5-carboxamide;
4-(4-hydroxycyclohexylamino)-2-(3,6,6-trimethyl-4-oxo-4,5,6,7-tetrahydro-1H-indol-1-yl)pyrimidine-5-carboxamide;
4-(2-hydroxycyclohexylamino)-2-(3,6,6-trimethyl-4-oxo-4,5,6,7-tetrahydro-1H-indol-1-yl)pyrimidine-5-carboxamide;
2-(5-carbamoyl-2-(3,6,6-trimethyl-4-oxo-4,5,6,7-tetrahydro-1H-indol-1-yl)pyrimidin-4-ylamino)cyclohexyl 2-aminoacetate;
4-(2-hydroxycyclopentylamino)-2-(3,6,6-trimethyl-4-oxo-4,5,6,7-tetrahydro-1H-indol-1-yl)pyrimidine-5-carboxamide;
4-(5-carbamoyl-2-(3,6,6-trimethyl-4-oxo-4,5,6,7-tetrahydro-1H-indol-1-yl)pyrimidin-4-ylamino)cyclohexyl 2-aminoacetate;
4-(1-methylpiperidin-4-ylamino)-2-(3,6,6-trimethyl-4-oxo-4,5,6,7-tetrahydro-1H-indol-1-yl)pyrimidine-5-carboxamide;
2-(5-carbamoyl-2-(3,6,6-trimethyl-4-oxo-4,5,6,7-tetrahydro-1H-indol-1-yl)pyrimidin-4-ylamino)cyclopentyl 2-aminoacetate;
4-(2-aminocyclohexylamino)-2-(3,6,6-trimethyl-4-oxo-4,5,6,7-tetrahydro-1H-indol-1-yl)pyrimidine-5-carboxamide;
3-(tetrahydrofuran-3-ylamino)-5-(3,6,6-trimethyl-4-oxo-4,5,6,7-tetrahydro-1H-indol-1-yl)pyrazine-2-carboxamide;
3-(tetrahydro-2H-pyran-4-ylamino)-5-(3,6,6-trimethyl-4-oxo-4,5,6,7-tetrahydro-1H-indol-1-yl)pyrazine-2-carboxamide;
3-(4-hydroxycyclohexylamino)-5-(3,6,6-trimethyl-4-oxo-4,5,6,7-tetrahydro-1H-indol-1-yl)pyrazine-2-carboxamide;
3-(2-hydroxycyclohexylamino)-5-(3,6,6-trimethyl-4-oxo-4,5,6,7-tetrahydro-1H-indol-1-yl)pyrazine-2-carboxamide;
2-(3-carbamoyl-6-(3,6,6-trimethyl-4-oxo-4,5,6,7-tetrahydro-1H-indol-1-yl)pyrazin-2-ylamino)cyclohexyl 2-aminoacetate;
3-(2-hydroxycyclopentylamino)-5-(3,6,6-trimethyl-4-oxo-4,5,6,7-tetrahydro-1H-indol-1-yl)pyrazine-2-carboxamide;
4-(3-carbamoyl-6-(3,6,6-trimethyl-4-oxo-4,5,6,7-tetrahydro-1H-indol-1-yl)pyrazin-2-ylamino)cyclohexyl 2-aminoacetate;
3-(1-methylpiperidin-4-ylamino)-5-(3,6,6-trimethyl-4-oxo-4,5,6,7-tetrahydro-1H-indol-1-yl)pyrazine-2-carboxamide;
2-(3-carbamoyl-6-(3,6,6-trimethyl-4-oxo-4,5,6,7-tetrahydro-1H-indol-1-yl)pyrazin-2-ylamino)cyclopentyl 2-aminoacetate;
3-(2-aminocyclohexylamino)-5-(3,6,6-trimethyl-4-oxo-4,5,6,7-tetrahydro-1H-indol-1-yl)pyrazine-2-carboxamide;
3-(tetrahydro-2H-thiopyran-4-ylamino)-5-(3,6,6-trimethyl-4-oxo-4,5,6,7-tetrahydro-1H-indol-1-yl)pyrazine-2-carboxamide;
3-(1-isobutylpiperidin-4-ylamino)-5-(3,6,6-trimethyl-4-oxo-4,5,6,7-tetrahydro-1H-indol-1-yl)pyrazine-2-carboxamide;
4-(tetrahydrofuran-3-ylamino)-2-(3,6,6-trimethyl-4-oxo-4,5,6,7-tetrahydro-1H-indazol-1-yl)pyrimidine-5-carboxamide;
4-(tetrahydro-2H-pyran-4-ylamino)-2-(3,6,6-trimethyl-4-oxo-4,5,6,7-tetrahydro-1H-indazol-1-yl)pyrimidine-5-carboxamide;
4-(4-hydroxycyclohexylamino)-2-(3,6,6-trimethyl-4-oxo-4,5,6,7-tetrahydro-1H-indazol-1-yl)pyrimidine-5-carboxamide;
4-(2-hydroxycyclohexylamino)-2-(3,6,6-trimethyl-4-oxo-4,5,6,7-tetrahydro-1H-indazol-1-yl)pyrimidine-5-carboxamide;
2-(5-carbamoyl-2-(3,6,6-trimethyl-4-oxo-4,5,6,7-tetrahydro-1H-indazol-1-yl)pyrimidin-4-ylamino)cyclohexyl 2-aminoacetate;
4-(2-hydroxycyclopentylamino)-2-(3,6,6-trimethyl-4-oxo-4,5,6,7-tetrahydroindazol-1-yl)pyrimidine-5-carboxamide;
4-(5-carbamoyl-2-(3,6,6-trimethyl-4-oxo-4,5,6,7-tetrahydro-1H-indazol-1-yl)pyrimidin-4-ylamino)cyclohexyl 2-aminoacetate;
4-(1-ethylpiperidin-4-ylamino)-2-(3,6,6-trimethyl-4-oxo-4,5,6,7-tetrahydro-1H-indazol-1-yl)pyrimidine-5-carboxamide;
2-(5-carbamoyl-2-(3,6,6-trimethyl-4-oxo-4,5,6,7-tetrahydro-1H-indazol-1-yl)pyrimidin-4-ylamino)cyclopentyl 2-aminoacetate;
4-(cyclohex-3-enylamino)-2-(3,6,6-trimethyl-4-oxo-4,5,6,7-tetrahydro-1H-indazol-1-yl)pyrimidine-5-carboxamide;
3-(tetrahydrofuran-3-ylamino)-5-(3,6,6-trimethyl-4-oxo-4,5,6,7-tetrahydro-1H-indazol-1-yl)pyrazine-2-carboxamide;
3-(tetrahydro-2H-pyran-4-ylamino)-5-(3,6,6-trimethyl-4-oxo-4,5,6,7-tetrahydro-1H-indazol-1-yl)pyrazine-2-carboxamide;
3-(4-hydroxycyclohexylamino)-5-(3,6,6-trimethyl-4-oxo-4,5,6,7-tetrahydro-1H-indazol-1-yl)pyrazine-2-carboxamide;
3-(2-hydroxycyclohexylamino)-5-(3,6,6-trimethyl-4-oxo-4,5,6,7-tetrahydro-1H-indazol-1-yl)pyrazine-2-carboxamide;
2-(3-carbamoyl-6-(3,6,6-trimethyl-4-oxo-4,5,6,7-tetrahydro-1H-indazol-1-yl)pyrazin-2-ylamino)cyclohexyl 2-aminoacetate;
3-(2-hydroxycyclopentylamino)-5-(3,6,6-trimethyl-4-oxo-4,5,6,7-tetrahydro-1H-indazol-1-yl)pyrazine-2-carboxamide;
4-(3-carbamoyl-6-(3,6,6-trimethyl-4-oxo-4,5,6,7-tetrahydro-1H-indazol-1-yl)pyrazin-2-ylamino)cyclohexyl 2-aminoacetate;
3-[(1,1-dioxidotetrahydro-2H-thiopyran-4-yl)amino]-5-(3,6,6-trimethyl-4-oxo-4,5,6,7-tetrahydro-1H-indazol-1-yl)pyrazine-2-carboxamide;
2-(3-carbamoyl-6-(3,6,6-trimethyl-4-oxo-4,5,6,7-tetrahydro-1H-indazol-1-yl)pyrazin-2-ylamino)cyclopentyl 2-aminoacetate;
3-(cyclopentylamino)-5-(3,6,6-trimethyl-4-oxo-4,5,6,7-tetrahydro-1H-indazol-1-yl)pyrazine-2-carboxamide;
2-(6,6-dimethyl-4-oxo-3-(trifluoromethyl)-4,5,6,7-tetrahydro-1H-indazol-1-yl)-4-(tetrahydrofuran-3-ylamino)pyrimidine-5-carboxamide;
2-(6,6-dimethyl-4-oxo-3-(trifluoromethyl)-4,5,6,7-tetrahydro-1H-indazol-1-yl)-4-(tetrahydro-2H-pyran-4-ylamino)pyrimidine-5-carboxamide;
2-(6,6-dimethyl-4-oxo-3-(trifluoromethyl)-4,5,6,7-tetrahydro-1H-indazol-1-yl)-4-(4-hydroxycyclohexylamino)pyrimidine-5-carboxamide;
2-(6,6-dimethyl-4-oxo-3-(trifluoromethyl)-4,5,6,7-tetrahydro-1H-indazol-1-yl)-4-(2-hydroxycyclohexylamino)pyrimidine-5-carboxamide;
2-(5-carbamoyl-2-(6,6-dimethyl-4-oxo-3-(trifluoromethyl)-4,5,6,7-tetrahydro-1H-indazol-1-yl)pyrimidin-4-ylamino)cyclohexyl 2-aminoacetate;
2-(6,6-dimethyl-4-oxo-3-(trifluoromethyl)-4,5,6,7-tetrahydro-1H-indazol-1-yl)-4-(2-hydroxycyclopentylamino)pyrimidine-5-carboxamide;
4-(5-carbamoyl-2-(6,6-dimethyl-4-oxo-3-(trifluoromethyl)-4,5,6,7-tetrahydro-1H-indazol-1-yl)pyrimidin-4-ylamino)cyclohexyl 2-aminoacetate;
4-(cyclopropylmethylamino)-2-(6,6-dimethyl-4-oxo-3-(trifluoromethyl)-4,5,6,7-tetrahydro-1H-indazol-1-yl)pyrimidine-5-carboxamide;
2-(5-carbamoyl-2-(6,6-dimethyl-4-oxo-3-(trifluoromethyl)-4,5,6,7-tetrahydro-1H-indazol-1-yl)pyrimidin-4-ylamino)cyclopentyl 2-aminoacetate;
4-(cyclopent-3-enylamino)-2-(6,6-dimethyl-4-oxo-3-(trifluoromethyl)-4,5,6,7-tetrahydro-1H-indazol-1-yl)pyrimidine-5-carboxamide;
5-(6,6-dimethyl-4-oxo-3-(trifluoromethyl)-4,5,6,7-tetrahydro-1H-indazol-1-yl)-3-(tetrahydrofuran-3-ylamino)pyrazine-2-carboxamide;
5-(6,6-dimethyl-4-oxo-3-(trifluoromethyl)-4,5,6,7-tetrahydro-1H-indazol-1-yl)-3-(tetrahydro-2H-pyran-4-ylamino)pyrazine-2-carboxamide;
5-(6,6-dimethyl-4-oxo-3-(trifluoromethyl)-4,5,6,7-tetrahydro-1H-indazol-1-yl)-3-(4-hydroxycyclohexylamino)pyrazine-2-carboxamide;
5-(6,6-dimethyl-4-oxo-3-(trifluoromethyl)-4,5,6,7-tetrahydro-1H-indazol-1-yl)-3-(2-hydroxycyclohexylamino)pyrazine-2-carboxamide;
2-(3-carbamoyl-6-(6,6-dimethyl-4-oxo-3-(trifluoromethyl)-4,5,6,7-tetrahydro-1H-indazol-1-yl)pyrazin-2-ylamino)cyclohexyl 2-aminoacetate;
5-(6,6-dimethyl-4-oxo-3-(trifluoromethyl)-4,5,6,7-tetrahydro-1H-indazol-1-yl)-3-(2-hydroxycyclopentylamino)pyrazine-2-carboxamide;
4-(3-carbamoyl-6-(6,6-dimethyl-4-oxo-3-(trifluoromethyl)-4,5,6,7-tetrahydro-1H-indazol-1-yl)pyrazin-2-ylamino)cyclohexyl 2-aminoacetate;
5-(6,6-dimethyl-4-oxo-3-(trifluoromethyl)-4,5,6,7-tetrahydro-1H-indazol-1-yl)-3-(2-oxotetrahydro-2H-pyran-3-ylamino)pyrazine-2-carboxamide;
2-(3-carbamoyl-6-(6,6-dimethyl-4-oxo-3-(trifluoromethyl)-4,5,6,7-tetrahydro-1H-indazol-1-yl)pyrazin-2-ylamino)cyclopentyl 2-aminoacetate;
5-(6,6-dimethyl-4-oxo-3-(trifluoromethyl)-4,5,6,7-tetrahydro-1H-indazol-1-yl)-3-(2-oxocyclohexylamino)pyrazine-2-carboxamide;
2-(3-ethyl-6,6-dimethyl-4-oxo-4,5,6,7-tetrahydro-1H-indazol-1-yl)-4-(tetrahydrofuran-3-ylamino)pyrimidine-5-carboxamide;
2-(3-ethyl-6,6-dimethyl-4-oxo-4,5,6,7-tetrahydro-1H-indazol-1-yl)-4-(tetrahydro-2H-pyran-4-ylamino)pyrimidine-5-carboxamide;
2-(3-ethyl-6,6-dimethyl-4-oxo-4,5,6,7-tetrahydro-1H-indazol-1-yl)-4-(4-hydroxycyclohexylamino)pyrimidine-5-carboxamide;
2-(3-ethyl-6,6-dimethyl-4-oxo-4,5,6,7-tetrahydro-1H-indazol-1-yl)-4-(2-hydroxycyclohexylamino)pyrimidine-5-carboxamide;
2-(3-ethyl-6,6-dimethyl-4-oxo-4,5,6,7-tetrahydro-1H-indazol-1-yl)-4-(2-hydroxycyclopentylamino)pyrimidine-5-carboxamide;
4-(5-carbamoyl-2-(3-ethyl-6,6-dimethyl-4-oxo-4,5,6,7-tetrahydro-1H-indazol-1-yl)pyrimidin-4-ylamino)cyclohexyl 2-aminoacetate;
2-(5-carbamoyl-2-(3-ethyl-6,6-dimethyl-4-oxo-4,5,6,7-tetrahydro-1H-indazol-1-yl)pyrimidin-4-ylamino)cyclohexyl 2-aminoacetate;
2-(5-carbamoyl-2-(3-ethyl-6,6-dimethyl-4-oxo-4,5,6,7-tetrahydro-1H-indazol-1-yl)pyrimidin-4-ylamino)cyclopentyl 2-aminoacetate;
4-(cyclopropylmethylamino)-2-(3,6,6-trimethyl-4-oxo-4,5,6,7-tetrahydro-1H-indazol-1-yl)pyrimidine-5-carboxamide;
4-(cyclopent-3-enylamino)-2-(3-ethyl-6,6-dimethyl-4-oxo-4,5,6,7-tetrahydro-1H-indazol-1-yl)pyrimidine-5-carboxamide;
5-(3-ethyl-6,6-dimethyl-4-oxo-4,5,6,7-tetrahydro-1H-indazol-1-yl)-3-(tetrahydrofuran-3-ylamino)pyrazine-2-carboxamide;
5-(3-ethyl-6,6-dimethyl-4-oxo-4,5,6,7-tetrahydro-1H-indazol-1-yl)-3-(tetrahydro-2H-pyran-4-ylamino)pyrazine-2-carboxamide;
5-(3-ethyl-6,6-dimethyl-4-oxo-4,5,6,7-tetrahydro-1H-indazol-1-yl)-3-(4-hydroxycyclohexylamino)pyrazine-2-carboxamide;
5-(3-ethyl-6,6-dimethyl-4-oxo-4,5,6,7-tetrahydro-1H-indazol-1-yl)-3-(2-hydroxycyclohexylamino)pyrazine-2-carboxamide;
5-(3-ethyl-6,6-dimethyl-4-oxo-4,5,6,7-tetrahydro-1H-indazol-1-yl)-3-(2-hydroxycyclopentylamino)pyrazine-2-carboxamide;
4-(3-carbamoyl-6-(3-ethyl-6,6-dimethyl-4-oxo-4,5,6,7-tetrahydro-1H-indazol-1-yl)pyrazin-2-ylamino)cyclohexyl 2-aminoacetate;
2-(3-carbamoyl-6-(3-ethyl-6,6-dimethyl-4-oxo-4,5,6,7-tetrahydro-1H-indazol-1-yl)pyrazin-2-ylamino)cyclohexyl 2-aminoacetate;
2-(3-carbamoyl-6-(3-ethyl-6,6-dimethyl-4-oxo-4,5,6,7-tetrahydro-1H-indazol-1-yl)pyrazin-2-ylamino)cyclopentyl 2-aminoacetate;
5-(3-ethyl-6,6-dimethyl-4-oxo-4,5,6,7-tetrahydro-1H-indazol-1-yl)-3-(2-oxocyclohexylamino)pyrazine-2-carboxamide;
5-(3-ethyl-6,6-dimethyl-4-oxo-4,5,6,7-tetrahydro-1H-indazol-1-yl)-3-(2-oxotetrahydro-2H-pyran-3-ylamino)pyrazine-2-carboxamide;
2-(3-(cyclopropylmethyl)-6,6-dimethyl-4-oxo-4,5,6,7-tetrahydro-1H-indazol-1-yl)-4-(tetrahydrofuran-3-ylamino)pyrimidine-5-carboxamide;
2-(3-(cyclopropylmethyl)-6,6-dimethyl-4-oxo-4,5,6,7-tetrahydro-1H-indazol-1-yl)-4-(tetrahydro-2H-pyran-4-ylamino)pyrimidine-5-carboxamide;
2-(3-(cyclopropylmethyl)-6,6-dimethyl-4-oxo-4,5,6,7-tetrahydro-1H-indazol-1-yl)-4-(4-hydroxycyclohexylamino)pyrimidine-5-carboxamide;
2-(3-(cyclopropylmethyl)-6,6-dimethyl-4-oxo-4,5,6,7-tetrahydro-1H-indazol-1-yl)-4-(2-hydroxycyclohexylamino)pyrimidine-5-carboxamide;
2-(3-(cyclopropylmethyl)-6,6-dimethyl-4-oxo-4,5,6,7-tetrahydro-1H-indazol-1-yl)-4-(2-hydroxycyclopentylamino)pyrimidine-5-carboxamide;
4-(5-carbamoyl-2-(3-(cyclopropylmethyl)-6,6-dimethyl-4-oxo-4,5,6,7-tetrahydro-1H-indazol-1-yl)pyrimidin-4-ylamino)cyclohexyl 2-aminoacetate;
2-(5-carbamoyl-2-(3-(cyclopropylmethyl)-6,6-dimethyl-4-oxo-4,5,6,7-tetrahydro-1H-indazol-1-yl)pyrimidin-4-ylamino)cyclohexyl 2-aminoacetate;
2-(5-carbamoyl-2-(3-(cyclopropylmethyl)-6,6-dimethyl-4-oxo-4,5,6,7-tetrahydro-1H-indazol-1-yl)pyrimidin-4-ylamino)cyclopentyl 2-aminoacetate;
4-(cyclohexylamino)-2-(3-(cyclopropylmethyl)-6,6-dimethyl-4-oxo-4,5,6,7-tetrahydro-1H-indazol-1-yl)pyrimidine-5-carboxamide;
2-(3-(cyclopropylmethyl)-6,6-dimethyl-4-oxo-4,5,6,7-tetrahydro-1H-indazol-1-yl)-4-(1,3-dimethoxypropan-2-ylamino)pyrimidine-5-carboxamide;
5-(3-(cyclopropylmethyl)-6,6-dimethyl-4-oxo-4,5,6,7-tetrahydro-1H-indazol-1-yl)-3-(tetrahydrofuran-3-ylamino)pyrazine-2-carboxamide;
5-(3-(cyclopropylmethyl)-6,6-dimethyl-4-oxo-4,5,6,7-tetrahydro-1H-indazol-1-yl)-3-(tetrahydro-2H-pyran-4-ylamino)pyrazine-2-carboxamide;
5-(3-(cyclopropylmethyl)-6,6-dimethyl-4-oxo-4,5,6,7-tetrahydro-1H-indazol-1-yl)-3-(4-hydroxycyclohexylamino)pyrazine-2-carboxamide;
5-(3-(cyclopropylmethyl)-6,6-dimethyl-4-oxo-4,5,6,7-tetrahydro-1H-indazol-1-yl)-3-(2-hydroxycyclohexylamino)pyrazine-2-carboxamide;
5-(3-(cyclopropylmethyl)-6,6-dimethyl-4-oxo-4,5,6,7-tetrahydro-1H-indazol-1-yl)-3-(2-hydroxycyclopentylamino)pyrazine-2-carboxamide;
4-(3-carbamoyl-6-(3-(cyclopropylmethyl)-6,6-dimethyl-4-oxo-4,5,6,7-tetrahydro-1H-indazol-1-yl)pyrazin-2-ylamino)cyclohexyl 2-aminoacetate;
2-(3-carbamoyl-6-(3-(cyclopropylmethyl)-6,6-dimethyl-4-oxo-4,5,6,7-tetrahydro-1H-indazol-1-yl)pyrazin-2-ylamino)cyclohexyl 2-aminoacetate;
2-(3-carbamoyl-6-(3-(cyclopropylmethyl)-6,6-dimethyl-4-oxo-4,5,6,7-tetrahydro-1H-indazol-1-yl)pyrazin-2-ylamino)cyclopentyl 2-aminoacetate;
5-(3-(cyclopropylmethyl)-6,6-dimethyl-4-oxo-4,5,6,7-tetrahydro-1H-indazol-1-yl)-3-(4-oxocyclohexylamino)pyrazine-2-carboxamide;
5-(3-(cyclopropylmethyl)-6,6-dimethyl-4-oxo-4,5,6,7-tetrahydro-1H-indazol-1-yl)-3-(3-hydroxycyclohexylamino)pyrazine-2-carboxamide;
2-(3-cyclopropyl-6,6-dimethyl-4-oxo-4,5,6,7-tetrahydro-1H-indazol-1-yl)-4-(4-hydroxycyclohexylamino)pyrimidine-5-carboxamide;
4-(4-hydroxycyclohexylamino)-2-(3-isopropyl-6,6-dimethyl-4-oxo-4,5,6,7-tetrahydro-1H-indazol-1-yl)pyrimidine-5-carboxamide;
4-(4-hydroxycyclohexylamino)-2-(3-isobutyl-6,6-dimethyl-4-oxo-4,5,6,7-tetrahydro-1H-indazol-1-yl)pyrimidine-5-carboxamide;
5-(3-cyclopropyl-6,6-dimethyl-4-oxo-4,5,6,7-tetrahydro-1H-indazol-1-yl)-3-(4-hydroxycyclohexylamino)pyrazine-2-carboxamide;
5-(3-(difluoromethyl)-6,6-dimethyl-4-oxo-4,5,6,7-tetrahydro-1H-indazol-1-yl)-3-(4-hydroxycyclohexylamino)pyrazine-2-carboxamide; or
5-(3-(fluoromethyl)-6,6-dimethyl-4-oxo-4,5,6,7-tetrahydro-1H-indazol-1-yl)-3-(4-hydroxycyclohexylamino)pyrazine-2-carboxamide; or a pharmaceutically acceptable salt of any of these compounds.
